SEQUENCE LISTING (142 sequences)

SEQ ID NO: 1 - protein, 108 aa, Mus sp.
QIVLTQSPAIMSASLGERVTMTCTASSSVSSSYLHWYQKKPGSSPKLWVYSTSNLASGVPVRFSGSGSGTSYSLTISSMEAEDAATYYCHQHHRSPVTFGSGTKLEMK

SEQ ID NO: 2 - protein, 107 aa, Mus sp.
DIQMTQSPASQSASLGESVTFTCLASQTIGTWLAWYQQRPGKSPQLLIYAATSLADGVPSRFSGSGSGTQFSFNIRSLQAEDFASYYCQQVYTTPLTFGGGTKLEIK

SEQ ID NO: 3 - protein, 107 aa, Mus sp.
DIQMTQSPASQSASLGESVTFTCLASQTIGTWLGWYQQKPGKSPQLLIYRSTTLADGVPSRFSGSGSGTKFSFKISSLQAADFASYYCQQLYSAPYTFGGGTKLEIR

SEQ ID NO: 4 - protein, 112 aa, Mus sp.
DVLLTQTPLSLPVSLGDQASISCRSSQNIVHSNGNTYLQWYLQKPGQSPKLLIYKVSNRFSGVPDRFSGSGSGTDFTLKISRVEAEDLGVYYCFQGSHVPFTFGAGTKLELK

SEQ ID NO: 5 - protein, 107 aa, Artificial Sequence
  Description of Artificial Sequence Synthetic polypeptide
DIQMTQTTSSLSASLGDRVTISCRASQDIYKYLNWYQQKPDGTLKLLIYYTSGLHSGVPSRFSGSGSGTDFSLTISNLEPEDIATYFCQQDSKFPWTFGGDTKLEIK

SEQ ID NO: 6 - protein, 108 aa, Mus sp.
QIVLTQSPAIMSASLGERVTMTCTASSSVSSSYFHWYQQKPGSSPKLWIYRTSNLASGVPGRFSGSGSGTSYSLTISSMEAEDAATYYCHQFHRSPLTFGAGTKLELK

SEQ ID NO: 7 - protein, 107 aa, Mus sp.
DIVMTQSQKFLSTSVGVRVSVTCKASQDVGTNVLWYQQKIGQSPKPLIYSASYRHSGVPDRFTGSGSGTDFTLIISNVQSEDLAEYFCQQYSRYPLTFGPGTKLELK

SEQ ID NO: 8 - protein, 107 aa, Mus sp.
DIVMTQSQKFLSTSVGVRVSVTCKASQDVGTNVLWYQQKIGQSPKALIYSASYRHSGVPDRFTGSGSGTDFTLIITNVQSEDLAEYFCQQYSRYPLTFGPGTKLELK

SEQ ID NO: 9 - protein, 107 aa, Mus sp.
DIVMTQSQKFMSATVGGRVNITCKASQNVGRAVAWYQQKPGQSPKLLTHSASNRYTGVPDRFTGSGSGTDFTLTITNMQSEDLADYFCQQYSSYPLTFGAGTKLDLK

SEQ ID NO: 10 - protein, 107 aa, Mus sp.
DIQMTQSPASQSASLGESVTFSCLASQTIGTWLGWYQQKPGKSPQLLIYRATSLADGVPSRFSGSGSGTNFSFKISSLQAEDLASYYCQQLYSGPYTFGGGTKLEIR

SEQ ID NO: 11 - protein, 119 aa, Mus sp.
QVQLQQSGTELLKPGASVKLSCKASGNTVTSYWMHWVKQRPGQGLEWIGEILPSTGRTNYNENFKGKAMLTVDKSSSTAYMQLSSLASEDSAVYYCTIVYFGNPWFAYWGQGTLVTVSA

SEQ ID NO: 12 - protein, 125 aa, Mus sp.
EVQLQQSGPELVKPGASVKLSCKASGYTFTDNYMNWVRQSHGKSLEWIGRVNPSNGDTKYNQNFKGKATLTVDKSLSTAYMQLNGLTSEDSAVYYCGRTKNFYSSYSYDDAMDYWGQGTSVTVSS

SEQ ID NO: 13 - protein, 124 aa, Mus sp.
EVQLQQSGAEFVRPGASVKFSCTASGFNIKDDYIHWVRQRPEQGLEWVGRIDPANGNTKYAPKFQDKATITADTSSNTAYLQLSSLTSEDTAVYYCAKSFPNNYYSYDDAFAYWGQGTLVTVSA

SEQ ID NO: 14 - protein, 118 aa, Mus sp.
QVQLKESGPVLVAPSQSLSITCTVSGFSLTKFGVHWIRQTPGKGLEWLGVIWAGGPTNYNSALMSRLTISKDISQSQVFLRIDSLQTDDTAMYYCAKQIYYSTLVDYWGQGTSVTVSS

SEQ ID NO: 15 - protein, 120 aa, Mus sp.
QVQLKESGPGLVAPSQSLFITCTVSGFSLSSYEINWVRQVPGKGLEWLGVIWTGITTNYNSALISRLSISKDNSKSLVFLKMNSLQTDDTAIYYCARGTGTGFYYAMDYWGQGTSVTVSS

SEQ ID NO: 16 - protein, 119 aa, Mus sp.
QVQLQQPGADFVRPGASMRLSCKASGYSFTSSWIHWVKQRPGQGLEWIGEINPGNVRTNYNENFRNKATLTVDKSSTTAYMQLRSLTSADSAVYYCTVVFYGEPYFPYWGQGTLVTVSA

SEQ ID NO: 17 - protein, 119 aa, Mus sp.
QVQLKESGPGLVAPSQSLSITCTVSGFSLTNYAVHWVRQFPGKGLEWLGVIWSDGSTDFNAPFKSRLSINKDNSKSQVFFKMNSLQIDDTAIYYCARKGGYSGSWFAYWGQGTLVTVSA

SEQ ID NO: 18 - protein, 119 aa, Mus sp.
QVQLKESGPGLVAPSQSLSITCTVSGFSLTNYAVHWVRQFPGKGLEWLGVIWSDGSTDYNAPFKSRLSINKDNSKSQVFFKMNSLQTDDTAIYYCARKGGYSGSWFAYWGQGTLVTVSA

SEQ ID NO: 19 - protein, 117 aa, Mus sp.
QVQLKESGPVLVAPSQSLSITCTVSGFSLTNYGVHWVRQPPGKGLEWLGVIWPVGSTNYNSALMSRLSIHKDNSKSQVFLRMNSLQTDDTAIYYCAKMDWDDFFDYWGQGTTLTVSS

SEQ ID NO: 20 - protein, 124 aa, Mus sp.
EVQLQQSGAELVRPGASVRLSCTASGFNIKDDYIHWVRQRPKQGLEWLGRIDPANGNTKYDPRFQDKATITADTSSNTAYLHLSSLTSEDTAVYYCAKSFPDNYYSYDDAFAYWGQGTLVTVSA

SEQ ID NO: 21 - protein, 12 aa, Mus sp.
TASSSVSSSYLH

SEQ ID NO: 22 - protein, 11 aa, Mus sp.
LASQTIGTWLA

SEQ ID NO: 23 - protein, 11 aa, Mus sp.
LASQTIGTWLG

SEQ ID NO: 24 - protein, 16 aa, Mus sp.
RSSQNIVHSNGNTYLQ

SEQ ID NO: 25 - protein, 11 aa, Mus sp.
RASQDIYKYLN

SEQ ID NO: 26 - protein, 12 aa, Mus sp.
TASSSVSSSYFH

SEQ ID NO: 27 - protein, 11 aa, Mus sp.
KASQDVGTNVL

SEQ ID NO: 28 - protein, 11 aa, Mus sp.
KASQNVGRAVA

SEQ ID NO: 29 - protein, 11 aa, Mus sp.
LASQTIGTWLG

SEQ ID NO: 30 - protein, 7 aa, Mus sp.
STSNLAS

SEQ ID NO: 31 - protein, 7 aa, Mus sp.
AATSLAD

SEQ ID NO: 32 - protein, 7 aa, Mus sp.
RSTTLAD

SEQ ID NO: 33 - protein, 7 aa, Mus sp.
KVSNRFS

SEQ ID NO: 34 - protein, 7 aa, Mus sp.
YTSGLHS

SEQ ID NO: 35 - protein, 7 aa, Mus sp.
RTSNLAS

SEQ ID NO: 36 - protein, 7 aa, Mus sp.
SASYRHS

SEQ ID NO: 37 - protein, 7 aa, Mus sp.
SASNRYT

SEQ ID NO: 38 - protein, 7 aa, Mus sp.
RATSLAD

SEQ ID NO: 39 - protein, 9 aa, Mus sp.
HQHHRSPVT

SEQ ID NO: 40 - protein, 9 aa, Artificial Sequence
  Description of Artificial Sequence Synthetic peptide
QQVYTTPLT

SEQ ID NO: 41 - protein, 9 aa, Mus sp.
QQLYSAPYT

SEQ ID NO: 42 - protein, 9 aa, Mus sp.
FQGSHVPFT

SEQ ID NO: 43 - protein, 9 aa, Mus sp.
QQDSKFPWT

SEQ ID NO: 44 - protein, 9 aa, Mus sp.
HQFHRSPLT

SEQ ID NO: 45 - protein, 9 aa, Mus sp.
QQYSRYPLT

SEQ ID NO: 46 - protein, 9 aa, Mus sp.
QQYSSYPLT

SEQ ID NO: 47 - protein, 9 aa, Mus sp.
QQLYSGPYT

SEQ ID NO: 48 - protein, 10 aa, Mus sp.
GNTVTSYWMH

SEQ ID NO: 49 - protein, 10 aa, Mus sp.
GYTFTDNYMN

SEQ ID NO: 50 - protein, 10 aa, Mus sp.
GFNIKDDYIH

SEQ ID NO: 51 - protein, 10 aa, Mus sp.
GFSLTKFGVH

SEQ ID NO: 52 - protein, 10 aa, Mus sp.
GFSLSSYEIN

SEQ ID NO: 53 - protein, 10 aa, Mus sp.
GYSFTSSWIH

SEQ ID NO: 54 - protein, 10 aa, Mus sp.
GFSLTNYAVH

SEQ ID NO: 55 - protein, 10 aa, Mus sp.
GFSLTNYGVH

SEQ ID NO: 56 - protein, 10 aa, Mus sp.
GFNIKDDYIH

SEQ ID NO: 57 - protein, 17 aa, Mus sp.
EILPSTGRTNYNENFKG

SEQ ID NO: 58 - protein, 17 aa, Mus sp.
RVNPSNGDTKYNQNFKG

SEQ ID NO: 59 - protein, 17 aa, Mus sp.
RIDPANGNTKYAPKFQD

SEQ ID NO: 60 - protein, 16 aa, Mus sp.
VIWAGGPTNYNSALMS

SEQ ID NO: 61 - protein, 16 aa, Mus sp.
VIWTGITTNYNSALIS

SEQ ID NO: 62 - protein, 15 aa, Mus sp.
EINPGNVRTNYNENF

SEQ ID NO: 63 - protein, 16 aa, Mus sp.
VIWSDGSTDFNAPFKS

SEQ ID NO: 64 - protein, 16 aa, Mus sp.
VIWSDGSTDYNAPFKS

SEQ ID NO: 65 - protein, 16 aa, Mus sp.
VIWPVGSTNYNSALMS

SEQ ID NO: 66 - protein, 17 aa, Mus sp.
RIDPANGNTKYDPRFQD

SEQ ID NO: 67 - protein, 10 aa, Mus sp.
VYFGNPWFAY

SEQ ID NO: 68 - protein, 16 aa, Mus sp.
TKNFYSSYSYDDAMDY

SEQ ID NO: 69 - protein, 15 aa, Mus sp.
SFPNNYYSYDDAFAY

SEQ ID NO: 70 - protein, 10 aa, Mus sp.
QIYYSTLVDY

SEQ ID NO: 71 - protein, 12 aa, Mus sp.
GTGTGFYYAMDY

SEQ ID NO: 72 - protein, 10 aa, Mus sp.
VFYGEPYFPY

SEQ ID NO: 73 - protein, 11 aa, Mus sp.
KGGYSGSWFAY

SEQ ID NO: 74 - protein, 9 aa, Mus sp.
MDWDDFFDY

SEQ ID NO: 75 - protein, 15 aa, Mus sp.
SFPDNYYSYDDAFAY

SEQ ID NO: 76 - protein, 108 aa, Artificial Sequence
  Description of Artificial Sequence Synthetic polypeptide
EIVLTQSPGTLSLSPGERATMSCTASSSVSSSYFHWYQQKPGQAPRLLIYRTSTLASGIPDRFSGSGSGTDFTLTISRLEPEDAATYYCHQFHRSPLTFGQGTKLEIK

SEQ ID NO: 77 - protein, 108 aa, Artificial Sequence
  Description of Artificial Sequence Synthetic polypeptide
EIVLTQSPGTLSLSPGERATMSCTASSSVSSSYFHWYQQKPGQAPRLLIYRTSILASGVPDRFSGSGSGTDFTLTISRLEPEDFATYYCHQFHRSPLTFGQGTKLEIK

SEQ ID NO: 78 - protein, 108 aa, Artificial Sequence
  Description of Artificial Sequence Synthetic polypeptide
EIVLTQSPGTLSLSPGERATMSCTASSSVSSSYFHWYQQKPGQAPRLWIYRTSRLASGVPDRFSGSGSGTDFTLTISRLEPEDAATYYCHQFHRSPLTFGQGTKLEIK

SEQ ID NO: 79 - protein, 108 aa, Artificial Sequence
  Description of Artificial Sequence Synthetic polypeptide
EIVLTQSPGTLSLSPGERATMTCTASSSVSSSYFHWYQQKPGQAPRLLIYRTSRLASGVPDRFSGSGSGTDFTLTISRLEPEDFAVYYCHQFHRSPLTFGQGTKLEIK

SEQ ID NO: 80 - protein, 108 aa, Artificial Sequence [sequence omitted]

SEQ ID NO: 81 - protein, 108 aa, Artificial Sequence
  Description of Artificial Sequence Synthetic polypeptide
QIVLTQSPGTLSLSPGERVTMSCTASSSVSSSYFHWYQQKPGQAPRLLIYRTSQLASGIPDRFSGSGSGTDFTLTISRLEPEDAATYYCHQFHRSPLTFGQGTKLEIK

SEQ ID NO: 82 - protein, 108 aa, Artificial Sequence
  Description of Artificial Sequence Synthetic polypeptide
QIVLTQSPGTLSLSPGERATMTCTASSSVSSSYFHWYQQKPGQAPRLLIYRTSKLASGVPDRFSGSGSGTDFTLTISRLEPEDFATYYCHQFHRSPLTFGQGTKLEIK

SEQ ID NO: 83 - protein, 108 aa, Artificial Sequence
  Description of Artificial Sequence Synthetic polypeptide
EIVLTQSPGTLSLSPGERATMSCTASSSVSSSYFHWYQQKPGQAPRLLIYRTSHLASGIPGRFSGSGSGTDFTLTISRLEPEDAAVYYCHQFHRSPLTFGQGTKLEIK

SEQ ID NO: 84 - protein, 107 aa, Artificial Sequence
  Description of Artificial Sequence Synthetic polypeptide
EIVMTQSPATLSVSPGVRATLSCKASQDVGTNVLWYQQKPGQAPRPLIYSASYRHSGIPDRFSGSGSGTEFTLTISSLQSEDFAEYFCQQYSRYPLTFGQGTKLEIK

SEQ ID NO: 85 - protein, 107 aa, Artificial Sequence
  Description of Artificial Sequence Synthetic polypeptide
EIVMTQSPATLSVSPGVRATLSCKASQDVGTNVLWYQQKPGQAPRPLIYSASYRHSGIPDRFSGSGSGTEFTLTISSLQSEDFAVYYCQQYSRYPLTFGQGTKLEIK

SEQ ID NO: 86 - protein, 107 aa, Artificial Sequence
  Description of Artificial Sequence Synthetic polypeptide
EIVMTQSPATLSVSPGVRATLSCKASQDVGTNVLWYQQKPGQAPRPLIYSASYRHSGIPARFSGSGSGTEFTLTISSLQSEDFAEYYCQQYSRYPLTFGQGTKLEIK

SEQ ID NO: 87 - protein, 119 aa, Artificial Sequence
  Description of Artificial Sequence Synthetic polypeptide
QVQLVQSGAEVKKPGASVKVSCKASGYSFTSSWIHWVRQAPGQGLEWIGEINPGNVRTNYNENFRNKATMTVDTSISTAYMELSRLRSDDTAVYYCAVVFYGEPYFPYWGQGTLVTVSS

SEQ ID NO: 88 - protein, 119 aa, Artificial Sequence
  Description of Artificial Sequence Synthetic polypeptide
QVQLVQSGAEVKKPGASVKVSCKASGYSFTSSWIHWVRQRPGQGLEWIGEINPGNVRTNYNENFRNRVTMTVDTSISTAYMELSRLRSDDTAVYYCTVVFYGEPYFPYWGQGTLVTVSS

SEQ ID NO: 89 - protein, 119 aa, Artificial Sequence [sequence omitted]

SEQ ID NO: 90 - protein, 119 aa, Artificial Sequence
  Description of Artificial Sequence Synthetic polypeptide
QVQLVQSGAEVKKPGASVKVSCKASGYSFTSSWIHWVRQRPGQGLEWMGEINPGNVRTNYNENFRNRATLTRDTSISTAYMELSRLRSDDTAVYYCAVVFYGEPYFPYWGQGTLVTVSS

SEQ ID NO: 91 - protein, 119 aa, Artificial Sequence
  Description of Artificial Sequence Synthetic polypeptide
QVQLVQSGAEVKKPGASVKVSCKASGYSFTSSWIHWVRQRPGQGLEWMGEILPGVVRTNYNENFRNKVTMTVDTSISTAYMELSRLRSDDTAVYYCTVVFYGEPYFPYWGQGTLVTVSS

SEQ ID NO: 92 - protein, 119 aa, Artificial Sequence
  Description of Artificial Sequence Synthetic polypeptide
QVQLVQSGAEVKKPGASVKVSCKASGYSFTSSWIHWVRQRPGQGLEWIGEINPGAVRTNYNENFRNRVTMTVDTSISTAYMELSRLRSDDTAVYYCTVVFYGEPYFPYWGQGTLVTVSS

SEQ ID NO: 93 - protein, 119 aa, Artificial Sequence
  Description of Artificial Sequence Synthetic polypeptide
QVQLVQSGAEVKKPGASVKVSCKASGYSFTSSWIHWVRQAPGQGLEWIGEINPGLVRTNYNENFRNKVTMTVDTSISTAYMELSRLRSDDTAVYYCAVVFYGEPYFPYWGQGTLVTVSS

SEQ ID NO: 94 - protein, 119 aa, Artificial Sequence
  Description of Artificial Sequence Synthetic polypeptide
QVQLVQSGAEVKKPGASVKVSCKASGYSFTSSWIHWVRQAPGQGLEWIGEINPGAVRTNYNENFRNKVTMTVDTSISTAYMELSRLRSDDTAVYYCAVVFYGEPYFPYWGQGTLVTVSS

SEQ ID NO: 95 - protein, 119 aa, Artificial Sequence
  Description of Artificial Sequence Synthetic polypeptide
QVQLVQSGAEVKKPGASVKVSCKASGYSFTSSWIHWVRQAPGQGLEWIGEINPGSVRTNYNENFRNKATMTVDTSISTAYMELSRLRSDDTAVYYCAVVFYGEPYFPYWGQGTLVTVSS

SEQ ID NO: 96 - protein, 119 aa, Artificial Sequence
  Description of Artificial Sequence Synthetic polypeptide
QVQLQESGPGLVKPSETLSITCTVSGFSLTDYAVHWIRQPPGKGLEWIGVIWSDGSTDYNAPFKSRVTINKDTSKSQVSFKMSSVQAADTAVYYCARKGGYSGSWFAYWGQGTLVTVSS

SEQ ID NO: 97 - protein, 119 aa, Artificial Sequence
  Description of Artificial Sequence Synthetic polypeptide
QVQLQESGPGLVKPSETLSITCTVSGFSLTDYAVHWIRQPPGKGLEWIGVIWSDGSTDYNAPFKSRVTISKDTSKNQVSLKMNSLTTDDTAVYYCARKGGYSGSWFAYWGQGTLVTVSS

SEQ ID NO: 98 - protein, 119 aa, Artificial Sequence
  Description of Artificial Sequence Synthetic polypeptide
QVQLQESGPGLVKPSETLSITCTVSGFSLTDYAVHWIRQPPGKGLEWIGVIWSDGSTDYNAPFKSRVTISKDNSKSQVSLKMNSVTVADTAVYYCARKGGYSGSWFAYWGQGTLVTVSS

SEQ ID NO: 99 - protein, 119 aa, Artificial Sequence
  Description of Artificial Sequence Synthetic polypeptide
QVQLQESGPGLVKPSETLSITCTVSGFSLTDYAVHWVRQPPGKGLEWIGVIWSDGSTDYNAPFKSRVTISKDTSKNQVSFKLSSVTVDDTAVYYCARKGGYSGSWFAYWGQGTLVTVSS

SEQ ID NO: 100 - protein, 119 aa, Artificial Sequence
  Description of Artificial Sequence Synthetic polypeptide
QVQLQESGPGLVAPSETLSLTCTVSGFSLTDYAVHWIRQFPGKGLEWIGVIWSDGSTDFNAPFKSRVTISKDTSKNQVSFKLSSVTTDDTAVYYCARKGGYSGSWFAYWGQGTLVTVSS

SEQ ID NO: 101 - protein, 119 aa, Artificial Sequence
  Description of Artificial Sequence Synthetic polypeptide
QVQLQESGPGLVKPSETLSITCTVSGFSLTDYAVHWIRQPPGKGLEWIGVIWSDGSTDYNAPFKSRVTISKDNSKSQVSFKMSSVTADDTAVYYCARKGGYSGSWFAYWGQGTLVTVSS

SEQ ID NO: 102 - protein, 7 aa, Mus sp.
RTSTLAS

SEQ ID NO: 103 - protein, 7 aa, Mus sp.
RTSILAS

SEQ ID NO: 104 - protein, 7 aa, Mus sp.
RTSRLAS

SEQ ID NO: 105 - protein, 7 aa, Mus sp.
RTSQLAS

SEQ ID NO: 106 - protein, 7 aa, Mus sp.
RTSKLAS

SEQ ID NO: 107 - protein, 10 aa, Mus sp.
GFSLTDYAVH

SEQ ID NO: 108 - protein, 15 aa, Mus sp.
EILPGVVRTNYNENF

SEQ ID NO: 109 - protein, 15 aa, Mus sp.
EINPGAVRTNYNENF

SEQ ID NO: 110 - protein, 15 aa, Mus sp.
EINPGLVRTNYNENF

SEQ ID NO: 111 - protein, 15 aa, Mus sp.
EINPGSVRTNYNENF

SEQ ID NO: 112 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 107 aa, Artificial Sequence [sequence omitted]

SEQ ID NO: 114 - protein, 215 aa, Artificial Sequence
  Description of Artificial Sequence Synthetic polypeptide
EIVLTQSPGTLSLSPGERATMSCTASSSVSSSYFHWYQQKPGQAPRLLIYRTSTLASGIPDRFSGSGSGTDFTLTISRLEPEDAATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 215 aa, Artificial Sequence
  Description of Artificial Sequence Synthetic polypeptide
EIVLTQSPGTLSLSPGERATMSCTASSSVSSSYFHWYQQKPGQAPRLLIYRTSILASGVPDRFSGSGSGTDFTLTISRLEPEDFATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 215 aa, Artificial Sequence
  Description of Artificial Sequence Synthetic polypeptide
EIVLTQSPGTLSLSPGERATMSCTASSSVSSSYFHWYQQKPGQAPRLWIYRTSRLASGVPDRFSGSGSGTDFTLTISRLEPEDAATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 215 aa, Artificial Sequence
  Description of Artificial Sequence Synthetic polypeptide
EIVLTQSPGTLSLSPGERATMTCTASSSVSSSYFHWYQQKPGQAPRLLIYRTSRLASGVPDRFSGSGSGTDFTLTISRLEPEDFAVYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - protein, 215 aa, Artificial Sequence
  Description of Artificial Sequence Synthetic polypeptide
QIVLTQSPGTLSLSPGERATMTCTASSSVSSSYFHWYQQKPGQAPRLWIYRTSRLASGVPDRFSGSGSGTDFTLTISRLEPEDAATYYCHQFHRSPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 215 aa, Artificial Sequence
  Description of Artificial Sequence Synthetic polypeptide
QIVLTQSPGTLSLSPGERVTMSCTASSSVSSSYFHWYQQKPGQAPRLLIYRTSQLASGIPDRFSGSGSGTDFTLTISRLEPEDAATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 215 aa, Artificial Sequence
  Description of Artificial Sequence Synthetic polypeptide
QIVLTQSPGTLSLSPGERATMTCTASSSVSSSYFHWYQQKPGQAPRLLIYRTSKLASGVPDRFSGSGSGTDFTLTISRLEPEDFATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 215 aa, Artificial Sequence
  Description of Artificial Sequence Synthetic polypeptide
EIVLTQSPGTLSLSPGERATMSCTASSSVSSSYFHWYQQKPGQAPRLLIYRTSHLASGIPGRFSGSGSGTDFTLTISRLEPEDAAVYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 214 aa, Artificial Sequence
  Description of Artificial Sequence Synthetic polypeptide
EIVMTQSPATLSVSPGVRATLSCKASQDVGTNVLWYQQKPGQAPRPLIYSASYRHSGIPDRFSGSGSGTEFTLTISSLQSEDFAEYFCQQYSRYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - protein, 214 aa, Artificial Sequence
  Description of Artificial Sequence Synthetic polypeptide
EIVMTQSPATLSVSPGVRATLSCKASQDVGTNVLWYQQKPGQAPRPLIYSASYRHSGIPDRFSGSGSGTEFTLTISSLQSEDFAVYYCQQYSRYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 124 - protein, 214 aa, Artificial Sequence
  Description of Artificial Sequence Synthetic polypeptide
EIVMTQSPATLSVSPGVRATLSCKASQDVGTNVLWYQQKPGQAPRPLIYSASYRHSGIPARFSGSGSGTEFTLTISSLQSEDFAEYYCQQYSRYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - protein, 449 aa, Artificial Sequence
  Description of Artificial Sequence Synthetic polypeptide
QVQLVQSGAEVKKPGASVKVSCKASGYSFTSSWIHWVRQAPGQGLEWIGEINPGNVRTNYNENFRNKATMTVDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 449 aa, Artificial Sequence
  Description of Artificial Sequence Synthetic polypeptide
QVQLVQSGAEVKKPGASVKVSCKASGYSFTSSWIHWVRQRPGQGLEWIGEINPGNVRTNYNENFRNRVTMTVDTSISTAYMELSRLRSDDTAVYYCTVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 449 aa, Artificial Sequence
  Description of Artificial Sequence Synthetic polypeptide
QVQLVQSGAEVKKPGASVKVSCKASGYSFTSSWIHWVKQAPGQGLEWMGEINPGNVRTNYNENFRNKVTMTVDTSISTAYMELSRLRSDDTAVYYCTVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 449 aa, Artificial Sequence
  Description of Artificial Sequence Synthetic polypeptide
QVQLVQSGAEVKKPGASVKVSCKASGYSFTSSWIHWVRQRPGQGLEWMGEINPGNVRTNYNENFRNRATLTRDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 449 aa, Artificial Sequence
  Description of Artificial Sequence Synthetic polypeptide
QVQLVQSGAEVKKPGASVKVSCKASGYSFTSSWIHWVRQRPGQGLEWMGEILPGVVRTNYNENFRNKVTMTVDTSISTAYMELSRLRSDDTAVYYCTVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 449 aa, Artificial Sequence
  Description of Artificial Sequence Synthetic polypeptide
QVQLVQSGAEVKKPGASVKVSCKASGYSFTSSWIHWVRQRPGQGLEWIGEINPGAVRTNYNENFRNRVTMTVDTSISTAYMELSRLRSDDTAVYYCTVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 449 aa, Artificial Sequence
  Description of Artificial Sequence Synthetic polypeptide
QVQLVQSGAEVKKPGASVKVSCKASGYSFTSSWIHWVRQAPGQGLEWIGEINPGLVRTNYNENFRNKVTMTVDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 449 aa, Artificial Sequence
  Description of Artificial Sequence Synthetic polypeptide
QVQLVQSGAEVKKPGASVKVSCKASGYSFTSSWIHWVRQAPGQGLEWIGEINPGAVRTNYNENFRNKVTMTVDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 449 aa, Artificial Sequence
  Description of Artificial Sequence Synthetic polypeptide
QVQLVQSGAEVKKPGASVKVSCKASGYSFTSSWIHWVRQAPGQGLEWIGEINPGSVRTNYNENFRNKATMTVDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 449 aa, Artificial Sequence
  Description of Artificial Sequence Synthetic polypeptide
QVQLQESGPGLVKPSETLSITCTVSGFSLTDYAVHWIRQPPGKGLEWIGVIWSDGSTDYNAPFKSRVTINKDTSKSQVSFKMSSVQAADTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 449 aa, Artificial Sequence
  Description of Artificial Sequence Synthetic polypeptide
QVQLQESGPGLVKPSETLSITCTVSGFSLTDYAVHWIRQPPGKGLEWIGVIWSDGSTDYNAPFKSRVTISKDTSKNQVSLKMNSLTTDDTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 449 aa, Artificial Sequence
  Description of Artificial Sequence Synthetic polypeptide
QVQLQESGPGLVKPSETLSITCTVSGFSLTDYAVHWIRQPPGKGLEWIGVIWSDGSTDYNAPFKSRVTISKDNSKSQVSLKMNSVTVADTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 449 aa, Artificial Sequence
  Description of Artificial Sequence Synthetic polypeptide
QVQLQESGPGLVKPSETLSITCTVSGFSLTDYAVHWVRQPPGKGLEWIGVIWSDGSTDYNAPFKSRVTISKDTSKNQVSFKLSSVTVDDTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 449 aa, Artificial Sequence
  Description of Artificial Sequence Synthetic polypeptide
QVQLQESGPGLVAPSETLSLTCTVSGFSLTDYAVHWIRQFPGKGLEWIGVIWSDGSTDFNAPFKSRVTISKDTSKNQVSFKLSSVTTDDTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 449 aa, Artificial Sequence
  Description of Artificial Sequence Synthetic polypeptide
QVQLQESGPGLVKPSETLSITCTVSGFSLTDYAVHWIRQPPGKGLEWIGVIWSDGSTDYNAPFKSRVTISKDNSKSQVSFKMSSVTADDTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 7 aa, Mus sp.
RTSHLAS

SEQ ID NO: 141 - protein, 5 aa, Mus sp.
SSWIH

SEQ ID NO: 142 - protein, 17 aa, Mus sp.
EINPGNVRTNYNENFRN

(19) United States
(12) Patent Application Publication
(10) Pub. No.: US 2022/0010022 A1
(43) Pub. Date: Jan. 13, 2022

(54) ANTI-IL-36R ANTIBODIES FOR THE TREATMENT OF ATOPIC DERMATITIS

(71) Applicant: Boehringer Ingelheim International GmbH, Ingelheim am Rhein (DE)

(72) Inventors: Mary Ruth FLACK, Ridgefield, CT (US); Jay FINE, Mount Kisco, NY (US); Janine LAMAR, Ingelheim am Rhein (DE); Steven John PADULA, Wiesbaden (DE); Chandrasena Reddy PAMULAPATI, Sandy Hook, CT (US); Meera RAMANUJAM, Danbury, CT (US); Ralf SIGMUND, Erbach (DE); Sudha VISVANATHAN, Hartsdale, NY (US); Elizabeta ZOVKO, Clifton, NJ (US)

(21) Appl. No.: 17/322,102

(22) Filed: May 17, 2021

Related U.S. Application Data
(60) Provisional application No. 63/026,774, filed on May 19, 2020.

(51) Int. Cl. C07K 16/28; A61P 17/00; A61K 45/06

(52) U.S. Cl. CPC C07K 16/2866; A61K 2039/505; A61K 45/06; A61P 17/00

(57) ABSTRACT

The present invention provides methods for treating, preventing or ameliorating atopic dermatitis (AtD). In certain embodiments, the invention provides methods to reduce skin infection in a patient with atopic dermatitis. The methods of the present invention include administering to a patient in need thereof a pharmaceutical composition including an anti-interleukin-36 receptor (anit-IL-36R) antibody.

### SEQUENCE LISTING

The instant application contains a Sequence Listing which has been submitted in ASCII format via EFS-Web and is hereby incorporated by reference in its entirety. Said ASCII copy, created on Aug. 26, 2021, is named 09-0702-US-2-SL.txt and is 146,408 bytes in size.

### TECHNICAL FIELD OF THE INVENTION

The present invention relates to the administration of an anti-interleukin-36 receptor (anit-IL-36R) antibody to the subject with atopic dermatitis (AtD) and the treatment and/or prevention of atopic dermatitis (AtD) in the subject. More specifically, the invention relates to the administration of spesolimab to a subject with AtD.

### BACKGROUND

Atopic dermatitis (AtD) is a chronic/relapsing inflammatory skin disease characterized by intense pruritus (e.g., severe itch) and by scaly and dry eczematous lesions. AtD is often associated with other atopic disorders such as allergic rhinitis and asthma. Patients with atopic dermatitis are susceptible to serious skin infections caused by bacteria and viruses including, but not limited to S. aureus and herpes simplex virus. S. aureus causes severe localized and diffuse (e.g., impetigo) skin infections. S. aureus colonization and infections of lesions significantly impacts AtD disease activity and severity.

Typical treatments include topical lotions and moisturizers, antibiotics, anti-viral and anti-fungal agents. Most treatment options, however, offer only temporary, incomplete, symptom relief. Moreover, in many patients with moderate-to-severe AtD, prolonged use of topical corticosteroids or calcineurin inhibitors may lead to increased risk of skin microbial infections. Thus, a need exists in the art for novel targeted therapies for the treatment and/or prevention of AtD.

### SUMMARY OF THE INVENTION

The present invention addresses the above need by providing biotherapeutics, in particular antibodies, which bind to IL-36R as a first- second-, third- or subsequent-line therapy for treating atopic dermatitis.

In one aspect, the present invention relates to a method for treating, preventing or ameliorating atopic dermatitis (AtD) in a subject, comprising administering to the subject a therapeutically effective amount of an anti-IL-36R antibody or an antigen-binding fragment thereof (as disclosed herein). In an embodiment relating to this aspect, the anti-IL-36R antibody is spesolimab. In another embodiment relating to this aspect, the anti-IL-36R antibody is administered according to any of the doses and dosage regimens provided in Tables 1 and 2.

In one aspect, the present invention relates to a method of reducing microbial colonization of the skin in a subject with atopic dermatitis comprising administering to the subject a therapeutically effective amount of an anti-IL-36-R antibody or an antigen-binding fragment thereof (as disclosed herein). In an embodiment relating to this aspect, the colonization is of a microbe selected from the group consisting of Staphylococcus aureus, Streptococcus spp., Pseudomonas aeruginosa, Bacteroides spp., molluscum contagiosum virus, Herpes simplex virus, coxsackievirus, vaccinia virus, Candida albicans, Microsporum spp., Trichophyton spp., Penicillium spp., Cladosporium spp., Alternaria spp., and Aspergillus spp. In another embodiment relating to this aspect, the microbe is Staphylococcus aureus (S. aureus). In another embodiment relating to this aspect, the S. aureus colonization is reduced by at least 10% or by at least 20% from the baseline following the administration of the anti-IL-36-R antibody or an antigen-binding fragment thereof as disclosed herein. In an embodiment relating to this aspect, the anti-IL-36R antibody is spesolimab. In another embodiment relating to this aspect, the anti-IL-36R antibody is administered according to any of the doses and dosage regimens provided in Tables 1 and 2.

In one aspect, the present invention relates to a method of reducing susceptibility to a skin infection in a subject with atopic dermatitis comprising administering to the subject a therapeutically effective amount of an anti-IL-36-R antibody or an antigen-binding fragment thereof (as disclosed herein). In an embodiment relating to this aspect, the skin infection is caused by a microbe selected from the group consisting of Staphylococcus aureus, Streptococcus spp., Pseudomonas aeruginosa, Bacteroides spp., Herpes simplex virus, molluscum contagiosum virus, coxsackievirus, vaccinia virus, Candida albicans, Microsporum spp., Trichophyton spp., Penicillium spp., Cladosporium spp., Alternaria spp., and Aspergillus spp. In another embodiment relating to this aspect, the microbe is Staphylococcus aureus (S. aureus). In an embodiment relating to this aspect, the anti-IL-36R antibody is spesolimab. In another embodiment relating to this aspect, the anti-IL-36R antibody is administered according to any of the doses and dosage regimens provided in Tables 1 and 2.

In one aspect, the present invention relates to a method of treating a skin disorder associated with AtD in a patient, said method(s) including administering or having administered to the patient a therapeutically effective amount of an anti-IL-36R antibody of the present invention or an antigen binding fragment thereof (as disclosed herein). In an embodiment relating to this aspect, the anti-IL-36R antibody is spesolimab. In another embodiment relating to this aspect, the anti-IL-36R antibody is administered according to any of the doses and dosage regimens provided in Tables 1 and 2.

In one aspect, the present invention relates to a method of treating skin inflammation associated with AtD in a subject, said method including administering or having administered to the subject a therapeutically effective amount of an anti-IL-36R antibody of the present invention or an antigen binding fragment thereof. In an embodiment relating to any of the above aspects, a second therapeutic agent is administered to the subject before, after, or concurrent with the anti-IL-36-R antibody or an antigen-binding fragment thereof. In a related embodiment, the second therapeutic agent is selected from the group consisting of an anti-bacterial agent, an anti-viral agent, an anti-fungal agent, another IL-36R antagonist, an IgE inhibitor, a corticosteroid, NSAID, an IL-4R antagonist, and IFNγ. In an embodiment relating to this aspect, the anti-IL-36R antibody is spesolimab. In another embodiment relating to this aspect, the anti-IL-36R antibody is administered according to any of the doses and dosage regimens provided in Tables 1 and 2.

In an embodiment relating to any of the above aspects, the anti-IL-36R antibody includes: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 53 or 141 (H-CDR1); the amino acid sequence of SEQ ID NO: 62, 108, 109, 110, 111 or 142 (H-CDR2); the amino acid sequence of SEQ ID NO: 72 (H-CDR3).

In an embodiment relating to any of the above aspects, the anti-IL-36R antibody includes: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 141 (H-CDR1); the amino acid sequence of SEQ ID NO: 62, 108, 109, 110, 111 or 142 (H-CDR2); the amino acid sequence of SEQ ID NO: 72 (H-CDR3).

In an embodiment relating to any of the above aspects, the anti-IL-36R antibody includes:
I. a) a light chain variable region comprising the amino acid sequence of SEQ ID NO: 26 (L-CDR1); the amino acid sequence of SEQ ID NO: 102 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
II. a) a light chain variable region comprising the amino acid sequence of SEQ ID NO: 26 (L-CDR1); the amino acid sequence of SEQ ID NO: 103 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
III. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
IV. a) a light chain variable region comprising the amino acid sequence of SEQ ID NO: 26 (L-CDR1); the amino acid sequence of SEQ ID NO: 105 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
V. a) a light chain variable region comprising the amino acid sequence of SEQ ID NO: 26 (L-CDR1); the amino acid sequence of SEQ ID NO: 106 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
VI. a) a light chain variable region comprising the amino acid sequence of SEQ ID NO: 26 (L-CDR1); the amino acid sequence of SEQ ID NO: 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
VII. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 141 (H-CDR1); the amino acid sequence of SEQ ID NO: 62, 108, 109, 110, 111 or 142 (H-CDR2); the amino acid sequence of SEQ ID NO: 72 (H-CDR3).

In an embodiment relating to any of the above aspects, the anti-IL-36R antibody includes:
(i) a light chain variable region comprising the amino acid sequence of SEQ ID NO: 77; and a heavy chain variable region comprising the amino acid sequence of SEQ ID NO: 87; or
(ii) a light chain variable region comprising the amino acid sequence of SEQ ID NO: 77; and a heavy chain variable region comprising the amino acid sequence of SEQ ID NO: 88; or
(iii) a light chain variable region comprising the amino acid sequence of SEQ ID NO: 77; and a heavy chain variable region comprising the amino acid sequence of SEQ ID NO: 89; or
(iv) a light chain variable region comprising the amino acid sequence of SEQ ID NO: 80; and a heavy chain variable region comprising the amino acid sequence of SEQ ID NO: 87; or
(v) a light chain variable region comprising the amino acid sequence of SEQ ID NO: 80; and a heavy chain variable region comprising the amino acid sequence of SEQ ID NO: 88; or
(vi) a light chain variable region comprising the amino acid sequence of SEQ ID NO: 80; and a heavy chain variable region comprising the amino acid sequence of SEQ ID NO: 89; or
(vii) a light chain variable region comprising the amino acid sequence of SEQ ID NO: 85; and a heavy chain variable region comprising the amino acid sequence of SEQ ID NO: 100; or
(viii) a light chain variable region comprising the amino acid sequence of SEQ ID NO: 85; and a heavy chain variable region comprising the amino acid sequence of SEQ ID NO:101; or
(ix) a light chain variable region comprising the amino acid sequence of SEQ ID NO: 86; and a heavy chain variable region comprising the amino acid sequence of SEQ ID NO: 100; or
(x) a light chain variable region comprising the amino acid sequence of SEQ ID NO: 86; and a heavy chain variable region comprising the amino acid sequence of SEQ ID NO:101.

In an embodiment relating to any of the above aspects, the anti-IL-36R antibody includes:
i. a light chain comprising the amino acid sequence of SEQ ID NO: 115; and a heavy chain comprising the amino acid sequence of SEQ ID NO: 125; or
ii. a light chain comprising the amino acid sequence of SEQ ID NO: 115; and a heavy chain comprising the amino acid sequence of SEQ ID NO: 126; or
iii. a light chain comprising the amino acid sequence of SEQ ID NO: 115; and a heavy chain comprising the amino acid sequence of SEQ ID NO: 127; or
iv. a light chain comprising the amino acid sequence of SEQ ID NO: 118; and a heavy chain comprising the amino acid sequence of SEQ ID NO: 125; or
v. a light chain comprising the amino acid sequence of SEQ ID NO: 118; and a heavy chain comprising the amino acid sequence of SEQ ID NO: 126; or
vi. a light chain comprising the amino acid sequence of SEQ ID NO: 118; and a heavy chain comprising the amino acid sequence of SEQ ID NO: 127; or
vii. a light chain comprising the amino acid sequence of SEQ ID NO: 123; and a heavy chain comprising the amino acid sequence of SEQ ID NO: 138; or
viii. a light chain comprising the amino acid sequence of SEQ ID NO: 123; and a heavy chain comprising the amino acid sequence of SEQ ID NO: 139; or
ix. a light chain comprising the amino acid sequence of SEQ ID NO: 124; and a heavy chain comprising the amino acid sequence of SEQ ID NO: 138.

In an embodiment relating to any of the aspects and embodiments described herein, the anti-IL-36R antibody is administered subcutaneously or intravenously or by both routes simultaneously or sequentially and in any order. In a related embodiment, the subcutaneous administration comprises administration of 300 mg or 600 mg of the anti-IL-36R antibody. In a related embodiment, the intravenous administration comprises administering 300 mg, 600 mg, 900 mg or 1200 mg of the anti-IL-36R antibody. In a related embodiment, the subcutaneous administration comprises administration of one or more doses of 300 mg or one or more doses of 600 mg each of the anti-IL-36R antibody once every week(qw), once every 2 weeks (q2w), once every 4 weeks (q4w), once every 6 weeks (q6w) or once every 8 weeks (q8w), or a combination thereof.

In another embodiment relating to any of the aspects and embodiments described herein, the anti-IL-36R antibody is administered subcutaneously or intravenously or by both routes simultaneously or sequentially and in any order. In a related embodiment, the subcutaneous administration comprises initial doses (e.g., a lead-in or an induction dose regime). In a related embodiment, the subcutaneous administration further comprises subsequent doses (e.g., maintenance dosage regimen). In a related embodiment, the initial doses includes: (a) one or more doses of 150 mg each of the anti-IL-36R antibody administered daily for 2 weeks; or (b) one or more doses of 300 mg each of the anti-IL-36R antibody administered daily for 2 weeks; or (c) one or more doses of 600 mg each of the anti-IL-36R antibody administered twice, three times or four times in 4 weeks or administered twice per week for 2 weeks, or administered twice per week for 3 weeks, or administered twice per week for 4 weeks; or (d) one dose of 900 mg or 1200 mg of the anti-IL-36R antibody administered once; or (e) two doses of 900 mg or 1200 mg each of the anti-IL-36R antibody administered twice in three weeks (e.g., in weeks 0 and 2); the subsequent dose includes: (a) one or more doses of 300 mg each of the anti-IL-36R antibody administered q2w, q4w, q6w or q8w; or (b) one or more doses of 600 mg each of the anti-IL-36R antibody administered q2w, q4w, q6w or q8w; and wherein the administration of the first subsequent dose is between 2 to 4 weeks or 2 weeks or 4 weeks after the administration of the last initial dose. In an embodiment, the administration of the first subsequent dose is between 2 to 4 weeks or 2 weeks or 4 weeks after the administration of the initial dose if only one initial dose is administered.

In another embodiment relating to any of the aspects and embodiments described herein, the anti-IL-36R antibody is administered subcutaneously at initial SC doses of 150 mg or 300 mg each (administered daily for 2 weeks) or 600 mg each (administered two times, three times, or four times in four weeks or twice per week in two weeks, three weeks or four weeks) or 900 mg or 1200 mg (administered once) or 900 mg or 1200 mg each (administered two times in three weeks) followed by subsequent SC doses of 300 mg or 600 mg (administered q2w, q4w, q6w or q8w). In an embodiment, the first subsequent dose is administered two to four weeks or two weeks or four weeks after the last initial dose. In an embodiment, the administration of the first subsequent dose is between two to four weeks or two weeks or four weeks after the administration of the initial dose if only one initial dose is administered.

In a related embodiment, the anti-IL-36R antibody administration results in one or more of the following outcomes over placebo or baseline:
i. at least 10% improvement in Eczema Area and Severity Index (EASI) score at 4 and/or 16 weeks;
ii. at least 10% improvement in proportion of patients who attain EASI 50 at 4 and/or 16 weeks;
iii. at least 10% improvement in proportion of patients who attain EASI 75 at 4 and/or 16 weeks;
iv. at least 10% improvement in SCORAD, Max Itch Intensity and DLQI;
v. at least 10% improvement in number of patients with drug related Adverse Events (AEs) up to week 44;
vi. at least 10% improvement in absolute and percentage change from baseline in Eczema Area and Severity Index (EASI) at week 4;
vii. at least 10% improvement in proportion of patients with a 50% improvement in Eczema Area and Severity Index (EASI)(EASI50) at weeks 4 and/or 16;
viii. at least 5% improvement in proportion of patients with a 75% improvement in Eczema Area and Severity Index (EASI)(EASI75) at week 4 and/or 16;
ix. at least 10% improvement in SCORing of Atopic Dermatitis (SCORAD) at week 4 and/or 16;
x. at least 5% improvement in proportion of patients achieving at least a 2-grade reduction to clear (0) or almost clear (1) in Investigator's Global Assessment (IGA) at week 4 and/or 16;
xi. at least 10 percentage point difference in percentage change from baseline in EASI score at week 16;
xii. at least 10 percentage point difference in EASI50 response rate at week 16;
xiii. at least 5 percentage point difference in EASI75 response rate at week 16;
xiv. at least 10 percentage point difference in percentage change from baseline in SCORAD, Max Itch Intensity and DLQI at week 16;
xv. at least 10 percentage point difference in percentage change from baseline in Eczema Area and Severity Index (EASI) at week 44; or
xvi. at least 5 percentage point difference in IGA rate at week 16.

In another embodiment relating to any of the above aspects and their related embodiments, the anti-IL-36R antibody is administered subcutaneously. In a related embodiment, the subcutaneous administration includes administration of one or more initial doses. In a related embodiment, the subcutaneous administration further includes administration of one or more subsequent doses. In a related embodiment, the initial doses are 150 mg, 300 mg, 600 mg, 900 mg or 1200 mg each administered according to an embodiment described herein. In a related embodiment, the initial doses of 150 mg or 300 mg are administered per day (in consecutive days) for two weeks. In a related embodiment, the initial doses of 600 mg each are administered once per week for two weeks including weeks 0 and 1; weeks 0 and 2; weeks 0 and 3; or weeks 0 and 4. In a related embodiment, the initial doses of 600 mg each are administered once per week for three weeks including weeks 0, 1 and 2; weeks 0, 1 and 3; weeks 0, 1 and 4; weeks 0, 2 and 3; weeks 0, 2 and 4; or weeks 0, 3 and 4. In a related embodiment, the initial doses of 600 mg each are administered once per week for four weeks including weeks 0, 1, 2 and 3; weeks 0, 1, 2 and 4; weeks 0, 1, 3 and 4; or weeks 0, 2, 3 and 4. In a related embodiment, the initial doses of 600 mg each are administered twice per week for 2 weeks. In a related embodiment, the initial doses of 600 mg each are administered twice per week for 3 weeks. In a related embodiment, the initial doses of 600 mg each are administered twice per week for 4 weeks. In a related embodiment, the initial dose of 900 mg or 1200 mg is administered once. In a related embodiment, the initial doses of 900 mg or 1200 mg each are administered twice in three weeks (e.g., in weeks 0 and 2). In a related embodiment, the subsequent doses include 300 mg or 600 mg of the anti-IL-36R. In a related embodiment, the subsequent dose administration begins two to four weeks or two weeks or four weeks after the initial dose administration ends. In a related embodiment, the subsequent doses of 300 mg or 600 mg each are administered q2w (once every 2 weeks), q4w (once every 4 weeks), q6w (once every 6 weeks) or q8w (once every 8 weeks). In a related embodiment, the anti-IL-36R antibody administration results in one or more of the following outcomes as compared to the placebo or baseline:
i. at least 10% improvement in Eczema Area and Severity Index (EASI) score at 4 and/or 16 weeks;
ii. at least 10% improvement in proportion of patients who attain EASI 50 at 4 and/or 16 weeks;
iii. at least 10% improvement in proportion of patients who attain EASI 75 at 4 and/or 16 weeks;
iv. at least 10% improvement in SCORAD, Max Itch Intensity and DLQI;
v. at least 10% improvement in number of patients with drug related Adverse Events (AEs) up to week 44;
vi. at least 10% improvement in absolute and percentage change from baseline in Eczema Area and Severity Index (EASI) at week 4;
vii. at least 10% improvement in proportion of patients with a 50% improvement in Eczema Area and Severity Index (EASI)(EASI50) at weeks 4 and/or 16;
viii. at least 5% improvement in proportion of patients with a 75% improvement in Eczema Area and Severity Index (EASI)(EASI75) at week 4 and/or 16;
ix. at least 10% improvement in SCORing of Atopic Dermatitis (SCORAD) at week 4 and/or 16;
x. at least 5% improvement in proportion of patients achieving at least a 2-grade reduction to clear (0) or almost clear (1) in Investigator's Global Assessment (IGA) at week 4 and/or 16;
xi. at least 10 percentage point difference in percentage change from baseline in EASI score at week 16;
xii. at least 10 percentage point difference in EASI50 response rate at week 16;
xiii. at least 5 percentage point difference in EASI75 response rate at week 16;
xiv. at least 10 percentage point difference in percentage change from baseline in SCORAD, Max Itch Intensity and DLQI at week 16;
xv. at least 10 percentage point difference in percentage change from baseline in Eczema Area and Severity Index (EASI) at week 44; or
xvi. at least 5 percentage point difference in IGA rate at week 16.

In an embodiment relating to any of the above aspects, the anti-IL-36R antibody or an antigen binding fragment thereof (disclosed herein) is present in a stable pharmaceutical formulation (as described in co-pending PCT application No. PCT/US2020/021059, filed Mar. 5, 2020, the entire content of which is hereby incorporated herein by reference in its entirety) for administration to a subject according to any one of the aspects of the present invention.

In one embodiment, the method of treatment according to any of the aspects described herein, includes administering to the subject a therapeutic amount of a stable pharmaceutical formulation comprising from about 20 mg/mL to about 150 mg/mL of an anti-IL-36R antibody (disclosed herein), about 20 mM to about 80 mM of a pharmaceutically acceptable buffer (e.g., acetate buffer), about 100 mM to about 250 mM of a pharmaceutically acceptable tonicifying agent (e.g., sucrose), about 0 mM to about 80 mM of a pharmaceutically acceptable stabilizing agent (e.g., arginine) or a pharmaceutically acceptable salt thereof, about 0 to about 150 mM of a pharmaceutically acceptable salt (e.g., sodium chloride), and a pharmaceutically acceptable surfactant (e.g., polysorbate 20) in an amount about 0 g/L to about 1.5 g/L, wherein the atopic dermatitis (AtD) in the subject is treated, prevented or ameliorated, wherein the microbial colonization of the skin in the subject with atopic dermatitis is reduced or inhibited, wherein the susceptibility to a skin infection in the subject with atopic dermatitis is reduced or inhibited, wherein the skin disorder associated with AtD in the subject is treated or prevented, wherein the skin inflammation associated with AtD in the subject is treated. In a related embodiment, the stable pharmaceutical formulation is an aqueous pharmaceutical formulation. In a related embodiment, the pH of the aqueous pharmaceutical formulation is about 5 to about 7. In a related embodiment, the pharmaceutical formulation is for an intravenous administration to the subject. In a related embodiment, the pharmaceutical formulation is for a subcutaneous administration to the subject. In a related embodiment, the pharmaceutical formulation for an intravenous administration comprises an anti-IL-36R antibody in an amount of about 60 mg/mL. In a related embodiment, the pharmaceutical formulation for a subcutaneous administration comprises an anti-IL-36R antibody in an amount of about 150 mg/mL.

It goes without saying that any of the herein disclosed methods, administration schemes and/or dosing regimens also equally apply to the use of any of the disclosed IL36-R antibodies in such methods, administration schemes and/or dosing regimens: i.e. an anti IL36R antibody, as disclosed herein, for use in the treatment, prevention and/or amelioration of any of the disclosed diseases and/or conditions. In other words, the invention also provides for the use of an anti IL36R antibody, as disclosed herein, for the manufacture of a medicament for the treatment, prevention and/or amelioration of any of the disclosed diseases and/or conditions.

Additional features and advantages of the present invention will-become apparent from a review of the ensuing detailed description-set forth below, and in part will be apparent from the description, or may be learned by practice of the subject technology. It is to be understood that both the foregoing general description and the following detailed description are exemplary and explanatory and are intended to provide further explanation of the present invention as claimed.

### BRIEF DESCRIPTION OF THE FIGURES

The accompanying drawings, which are included to provide further understanding of the present invention and are incorporated in and constitute a part of this specification, illustrate aspects of the subject technology and together with the description serve to explain the principles of the present invention.

FIG. 1 shows the IL-36 antagonist ligands (IL-36RA/IL1F5, IL-38/ILF10) inhibiting the signaling cascade.

FIG. 2 shows formalin fixed paraffin embedded (FFPE) skin biopsies from atopic dermatitis and non-AtD healthy controls (using ISH probes) stained for IL-36 α,β,γ and IL-36R.

FIG. 3 shows that systemic administration (intraperitoneal) of a mouse anti-IL-36R blocking mAb reduces the disease score in mouse S. aureus skin inflammation model.

FIG. 4 shows that systemic administration (intraperitoneal) of a mouse anti-IL36R blocking mAb reduces the epidermal thickness in a mouse S. aureus skin inflammation model.

FIG. 5 shows the change from baseline (%) in EASI score up to Week 16 in patients who received 600 mg (iv) spesolimab (once every four weeks (q4w) starting on week 0 and ending on week 12) versus the placebo patients—MMRM estimates (OC-MI)—FAS—as discussed in Example 7. MMRM stands for Mixed Model Repeated measures. MI stands for Multiple Imputations. OC stands for Observed cases, which means that data were not set to missing or anything else.

FIG. 6 shows the change from baseline (%) in EASI score up to Week 16 (MMRM OC FAS w/o CS) in a subset of patients who were not on corticosteroids (CS) during the trial period in both the spesolimab and placebo arms. FAS stands for full analysis. FAS w/o CS means that the data from patients who used CS concomitantly during the trial were excluded from the analysis.

FIG. 7 shows the change from baseline (%) in EASI score up to the end of the trial—observed values in FAS patients who continued after re-allocation period. The top line patients received 8 doses of spesolimab. The bottom line received only four doses, at Weeks 0, 4, 8 and 12. Solid or closed circle represents data from 5 patients who were initially randomized to spesolimab and received four doses of spesolimab on Weeks 0, 4, 8 and 12 but did not receive open-label spesolimab treatment at Week 16 until Week 28. Open circle represents data from 16 non-responder patients who were initially randomized to spesolimab on Weeks 0, 4, 8 and 12 and were re-allocated to receive four more doses of open label spesolimab at week 16 to week 28.

### DETAILED DESCRIPTION OF THE INVENTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

In the following detailed description, numerous specific details are set forth to provide a full understanding of the present invention. It will be apparent, however, to one ordinarily skilled in the art that the subject technology may be practiced without some of these specific details. In other instances, well-known structures and techniques have not been shown in detail so as not to obscure the present invention.

Unless defined otherwise, all technical and scientific terms used herein have the same meaning as commonly understood by one of ordinary skill in the art to which this invention belongs.

The present invention includes methods for reducing susceptibility to a skin infection in a subject with AtD by administering to the subject a therapeutically effective amount of an anti-IL-36R antibody or an antigen binding fragment thereof.

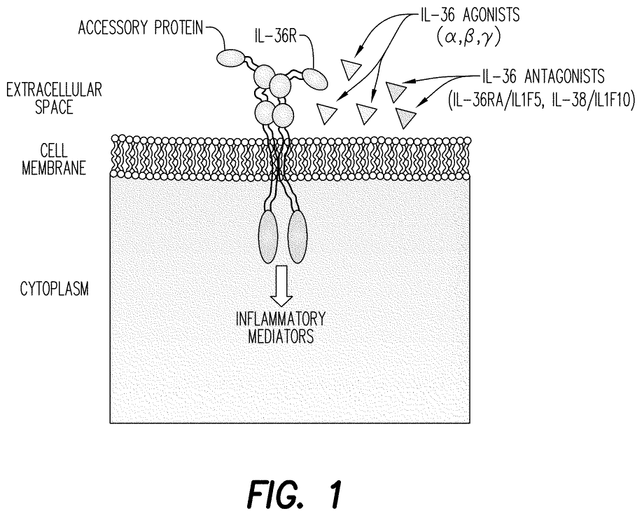

Without wishing to be bound by this theory it is believed that anti-IL-36R antibodies or antigen-binding fragments thereof bind to human IL-36R and thus interfere with the binding of IL-36 agonists, and in doing so block at least partially the signaling cascade from the IL-36R to inflammatory mediators. This is illustrated by FIG. 1. IL-36R is also known as IL-1 RL2 and IL-1 Rrp2. It has been reported that agonistic IL-36 ligands (α, β, or γ) initiate the signaling cascade by engaging the IL-36 receptor which then forms a heterodimer with the IL-1 receptor accessory protein (IL-1 RAcP).

The anti-IL36R antibodies of the present invention are disclosed herein an in, for example, in U.S. Pat. No. 9,023,995, the entire content of which is incorporated herein by reference.

### Definitions

A phrase such as “an aspect” does not imply that such aspect is essential to the present invention or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa. A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure.

As used herein, the terms “treat”, “treating”, or the like, mean to alleviate symptoms, eliminate the causation of symptoms either on a temporary or permanent basis, or to prevent or slow the appearance of symptoms of the named disorder or condition.

The term “about” shall generally mean an acceptable degree of error or variation for the quantity measured given the nature or precision of the measurements. Typical, exemplary degrees of error or variation are within 5% or within 3% or within 1% of a given value or range of values. For example, the expression of “about 100” includes 105 and 95 or 103 and 97 or 101 and 99, and all values in between (e.g., 95.1, 95.2, etc. for range of 95-105; or 97.1, 97.2, etc. for the range of 97-103; 99.1, 99.2, etc. for the range of 99-101). Numerical quantities given herein are approximates unless stated otherwise, meaning that the term “about” can be inferred when not expressly stated.

As used herein, the term “pharmaceutical formulation” or “formulation” refers to the process but also the product of a process in which an active drug or agent is combined with chemical substances to produce a final medicinal or drug product, the final formulation therefore refers to medicinal products such as liquids, powders or compositions. Therefore, in one embodiment, a pharmaceutical formulation is a pharmaceutical composition.

A “pharmaceutical composition” refers in this context to a liquid or powder preparation which is in such form as to permit the biological activity of the active ingredient(s) to be unequivocally effective, and which contains no additional components which are significantly toxic to the subjects to which the composition would be administered. Such compositions are sterile. A “powder” refers to a freeze-dried or lyophilized or a spray-dried pharmaceutical composition for parenteral use. The powder is reconstituted or dissolved typically in water. Lyophilisation is a low temperature dehydration process which involves freezing the product, lowering pressure, then removing the ice by sublimation. Freeze drying results in a high quality product because of the low temperature used in processing. For a well-developed lyophilized formulation, the shape and appearance of the product is maintained over time and the quality of the rehydrated product is excellent. Spray drying is a method of producing a dry powder from a liquid or slurry by rapidly drying with a hot gas and with the goal of achieving a consistent particle size distribution.

The terms “initial dose(s),” “subsequent dose(s),” refer to the temporal sequence of administration of the anti-IL-36R antibody. Thus, the “initial dose(s)” include one or more doses administered at the beginning of the treatment period, e.g., within the first four weeks of the treatment; the “subsequent doses” include one or more doses administered after the initial dose. A first dose of the subsequent doses is normally administered about 2 to 4 weeks (e.g., 2 weeks or 4 weeks) after the last dose of the initial doses. The initial and subsequent dose(s) may all contain the same amount of anti-IL-36R antibody or an antigen binding fragment thereof, but generally may differ from one another in terms of the amount of the antibody administered or the frequency of administration. In certain embodiments, however, the amount of the anti-IL-36R antibody contained in the initial, subsequent doses varies from one another during the course of treatment. In certain embodiments, the one or more initial doses each comprise a first amount of the antibody or antigen-binding fragment thereof and the one or more subsequent doses each comprise a second amount of the antibody or antigen-binding fragment thereof. In some embodiments, the first amount or initial dose of the antibody or fragment thereof is 1.5×, 2×, 2.5×, 3×, 3.5×, 4×, or 5× the subsequent amount/dose of the antibody or antigen-binding fragment thereof. In certain embodiments, one or more (e.g., 1, 2, 3, 4, or 5 or more) initial doses are administered at the beginning of the treatment regimen as “loading dose(s)” or “leading dose(s)” followed by subsequent doses that may be administered on a less frequent basis (e.g., “maintenance dose(s)”). For example, an anti-IL-36R antibody may be administered to a subject with AtD at one or more initial doses (or loading doses or leading doses) of about 150 mg, about 300 mg, about 600 mg, about 900 mg or about 1200 mg each followed by one or more subsequent doses (or maintenance doses) of about 300 mg or 600 mg each of the anti-IL-36R antibody.

As used herein “buffer” refers to a buffered solution that resists changes in pH by the action of its acid-base conjugate components. The “pH” herein refers to the acidity or basicity of the composition at room temperature. Standard methods to measure the pH of a composition are known to the skilled in the art. Typically, measuring pH consists of calibrating the instrument, placing the electrodes in a well-mixed sample, and then reading the pH directly from the pH meter. The exemplary buffers of the present invention include acetate, citrate, histidine, succinate, phosphate and Tris.

As used herein, the term “tonicifying agent” or “tonicity agent” or “tonicifyer” refers to substances providing an osmotic pressure equivalent to that of serum in the body including salts (e.g. sodium chloride, potassium chloride, magnesium chloride) or sugars (e.g. sucrose, trehalose, sorbitol, magnesium sulfate (MgSO₄), glycerol, mannitol or dextrose). In addition, sugars present in the solution act as a cryoprotectant for the protein which allows the drug substance to be frozen without damage. This permits shipment in the frozen form and long-term storage of the drug substance prior to the filling of drug product. The exemplary tonicifying agents of the present invention include sodium chloride, potassium chloride, magnesium chloride (salts) and/or sucrose, trehalose, sorbitol, magnesium sulfate (MgSO₄), glycerol, man nitol or dextrose (sugars).

As used herein, the term “stabilizer” or “stabilizing agent” refers to substances contributing to the stability of the active ingredient in a pharmaceutical formulation. The exemplary stabilizing agents of the present invention include arginine, histidine, glycine, cysteine, proline, methionine, lysine, or pharmaceutically acceptable salts thereof.

As used herein, the term “surfactant” refers to substances which tend to reduce the surface tension of a liquid in which they are dissolved. The exemplary surfactants of the present invention include poloxamer 188, polysorbate 20, polysorbate 40, polysorbate 60 or polysorbate 80.

The term “subcutaneous administration” refers to introduction of an agent under the skin of an animal or human patient, preferable within a pocket between the skin and underlying tissue, by relatively slow, sustained delivery from a drug receptacle. Pinching or drawing the skin up and away from underlying tissue may create the pocket.

The term “subject” for purposes of treatment refers to any animal classified as a mammal, including humans, domesticated and farm animals, and zoo, sports, or pet animals, such as dogs, horses, cats, cows, and the like. Preferably, the mammal is human.

The terms “treatment” and “therapy” and the like, as used herein, are meant to include therapeutic as well as prophylactic, or suppressive measures for a disease or disorder leading to any clinically desirable or beneficial effect, including but not limited to alleviation or relief of one or more symptoms, regression, slowing or cessation of progression of the disease or disorder. Thus, for example, the term treatment includes the administration of an agent prior to or following the onset of a symptom of a disease or disorder thereby preventing or removing one or more signs of the disease or disorder. As another example, the term includes the administration of an agent after clinical manifestation of the disease to combat the symptoms of the disease. Further, administration of an agent after onset and after clinical symptoms have developed where administration affects clinical parameters of the disease or disorder, such as the degree of tissue injury or the amount or extent of metastasis, whether or not the treatment leads to amelioration of the disease, comprises “treatment” or “therapy” as used herein. Moreover, as long as the compositions of the invention either alone or in combination with another therapeutic agent alleviate or ameliorate at least one symptom of a disorder being treated as compared to that symptom in the absence of use of the humanized anti-IL-36R antibody composition, the result should be considered an effective treatment of the underlying disorder regardless of whether all the symptoms of the disorder are alleviated or not.

The term “therapeutically effective amount” is used to refer to an amount of an active agent that relieves or ameliorates one or more of the symptoms of the disorder being treated. In another aspect, the therapeutically effective amount refers to a target serum concentration that has been shown to be effective in, for example, slowing disease progression. Efficacy can be measured in conventional ways, depending on the condition to be treated.

The term “prophylactically effective amount” is used to refer to an amount effective, at dosages and for periods of time necessary, to achieve the desired prophylactic result. Typically, a prophylactic dose is used in subjects prior to the onset of symptoms of AtD such as to prevent or inhibit the occurrence of acute symptoms. In an embodiment, a subcutaneous dose as contemplated herein may be a prophylactic dose that is used in a patient with AtD, after the initial or induction dose, to prevent a possible recurrence of the AtD symptoms in the patient.

The term “package insert” is used to refer to instructions customarily included in commercial packages of therapeutic products, that contain information about the indications, usage, administration, contraindications and/or warnings concerning the use of such therapeutic products.

Although any methods and materials similar or equivalent to those described herein can be used in the practice of the present invention, the preferred methods and materials are now described. All publications mentioned herein are incorporated herein by reference to describe in their entirety.

### Methods for Treating, Preventing or Ameliorating Skin Infections

Atopic dermatitis (AtD) is a common skin disease with a complex evolving pathogenesis. AtD can often begin in early childhood which continues to adulthood or develops newly in adults. The prevalence of the disease continues to rise with data suggesting that one-quarter to one third of individuals will be affected in the Unites states (Sullivan and Silverberg, 2017). Several factors contribute to the pathogenesis of AtD including environmental and genetic factors that drive the activation of immune cells and their migration to the skin, and barrier abnormalities. The microbiota of the skin is important in maintaining immune homeostasis and preventing the growth of pathogens such as S. aureus. During AtD flare the diversity of the normal microflora is diminished, allowing S. aureus to proliferate, in part encouraged by a reduction in bacteria with anti-S. aureus activity. S. aureus elaborates several molecules with potential to cause inflammation and to promote further immune dysregulation (Geoghean et al., 2017). While there is strong link between AtD pathophysiology and deregulated Th2 responses, emerging data suggests that additional disease drivers distinct from direct Th2-linked mechanisms such as Th17, Th22 and innate drivers could be relevant and may offer opportunities for therapeutic intervention (Esaki et al., 2015). The evolution of these additional components of AtD pathogenesis has led to questioning the origin of disease and whether it is triggered by external or internal factors (i.e., the outside in versus inside out hypotheses) (Silverberg and Silverberg, 2015).

IL36R is a novel member of the IL1R family that forms a heterodimeric complex with the IL1R accessory protein (IL1 RAcp) and IL1 Rrp2 associated with epithelial mediated inflammation and barrier dysfunction. The heterodimeric IL36R system with stimulating (IL36α, IL36β, IL36γ) and inhibitory ligands (IL36Ra and IL38) shares a number of structural and functional similarities to other members of the IL1/ILR family, such as IL1, IL18 and IL33. All IL1 family members (IL1α, IL1β, IL18, IL36α, IL36β, IL36γ, and IL38) signal through a unique, cognate receptor protein which, upon ligand binding, recruits the common IL1 RAcP subunit and activates NFκB and MAP kinase pathways in receptor-positive cell types (Dinarello, 2011; Towne et al., 2004; Towne et al., 2011). Genetic human studies have established a strong link between IL36R signaling and skin inflammation as demonstrated by occurrence of generalized pustular psoriasis in patients with a loss of function mutation in IL36Ra which results in uncontrolled IL36R signaling (Marrakchi et al., 2011).

IL36R is expressed in epithelial cells (e.g. keratinocytes, intestinal epithelial cells), dermal fibroblasts, and immune cells (myeloid cells, B cells and T cells). The link between IL36R and atopic dermatitis pathogenesis is emerging. Increased expression of IL36a and IL36γ as well as IL36Ra has been demonstrated in lesion tissue from AtD patients (D'Erme et al., 2015; Suarez-Farinas et al., 2015)). In vivo, IL36R signaling promotes the inflammatory response induced by epicutaneous challenge with Staphylococcus aureus (Liu et al, 2017). Mice deficient of IL36R receptor had significantly reduced skin inflammation and keratinocyte proliferation compared to wildtype controls, without augmentation of S. aureus colonization. These observations were restricted to IL36R pathway since no impact on S. aureus induced inflammation was observed in mice deficient of other IL1 family of cytokines; IL1α-KO, IL1β-KO or IL33-KO. The cellular mechanism of this reduced IL36R-dependent skin inflammation was through a reduction in both IL17 and IL22 from infiltrating T-cells. Given the strong connection between S. aureus colonization and severity of AtD disease it is compelling to consider that IL36R biology may contribute to AtD pathophysiology and hence blocking IL36R activation will be beneficial in patients suffering from AtD.

Therefore, the present invention includes methods which comprise administering to a subject in need thereof a therapeutically effective amount of an anti-IL-36R antibody or an antigen binding fragment thereof. As used herein, the expression “a subject in need thereof” means a human or a non-human animal that exhibits one or more symptoms of atopic dermatitis, e.g., skin infection, and/or who has been diagnosed with AtD. In an embodiment, the skin infection is selected from the group consisting of impetigo, cellulitis, infected dermatitis, eczema herpeticum, folliculitis, infected blister, mycosis, tinea versicolor, Staphylococcus aureus infection, and Streptococcus infection. A microbe that cause the infection includes, but is not limited to Staphylococcus aureus, Streptococcus spp., Pseudomonas aeruginosa, Bacteroides spp., Herpes simplex virus, coxsackievirus, molluscum contagiosum virus, vaccinia virus, Candida albicans, Microsporum spp., Trichophyton spp., Penicillium spp., Cladosporium spp., Alternaria spp., and Aspergillus spp. The term “a subject in need thereof” may also refer to a subject with AtD and with increased susceptibility to a skin infection or at greater risk of developing a skin infection. It may also include a subject with elevated levels of serum total and allergen-specific IgE, or serum chemokines (e.g., CCL17 or CCL27).

In certain aspects, the methods of the invention may be used to reduce inflammation, and/or pruritus due to AtD.

The present invention provides methods to reduce microbial colonization of the skin in a subject with AtD comprising administering a therapeutically effective amount of an anti-IL-36R antibody or an antigen binding fragment thereof to the subject. In certain embodiments, the invention provides for methods to reduce colonization of S. aureus on the skin of patients with atopic dermatitis. In some embodiments, the microbial colonization is reduced by at least 10%, at least 15%, at least 20%, at least 25%, at least 30%, at least 35%, at least 40%, at least 45%, at least 50%, at least 55%, at least 60%, at least 65%, at least 70%, or at least 75% as compared to the baseline, upon administration of the anti-IL-36R antibody.

Microbial colonization may be measured with tests and procedures known in the art, e.g., by PCR, microbial culture, microscopy and staining or immunofluorescence. In certain embodiments, microbial colonization may be measured by the presence of microbial protein biomarkers known in the art, e.g., microbial toxin such as staph toxic shock syndrome toxin-1. Methods for detecting and/or quantifying such biomarkers are known in the art.

### Antibodies of the Present Invention

The anti-IL36R antibodies of the present invention are disclosed in U.S. Pat. No. 9,023,995 or WO2013/074569, the entire content of each of which is incorporated herein by reference.

The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of the anti-IL-36R antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

The antibodies used in the methods of the present invention may be human antibodies. The term “human antibody,” as used herein, is intended to include antibodies having variable and constant regions derived from human germline immunoglobulin sequences. The human antibodies of the invention may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods of the present invention may be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

In certain exemplary embodiments related to any aspects of the present invention, the anti-IL-36R antibody or antigen-binding fragment thereof that can be used in the context of the methods of the present invention includes: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 53 or 141 (H-CDR1); the amino acid sequence of SEQ ID NO: 62, 108, 109, 110, 111 or 142 (H-CDR2); the amino acid sequence of SEQ ID NO: 72 (H-CDR3).

According to certain embodiments, the anti-IL-36R antibody or antigen-binding fragment thereof comprises:
I. a) a light chain variable region comprising the amino acid sequence of SEQ ID NO: 26 (L-CDR1); the amino acid sequence of SEQ ID NO: 102 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
II. a) a light chain variable region comprising the amino acid sequence of SEQ ID NO: 26 (L-CDR1); the amino acid sequence of SEQ ID NO: 103 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
III. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
IV. a) a light chain variable region comprising the amino acid sequence of SEQ ID NO: 26 (L-CDR1); the amino acid sequence of SEQ ID NO: 105 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
V a) a light chain variable region comprising the amino acid sequence of SEQ ID NO: 26 (L-CDR1); the amino acid sequence of SEQ ID NO: 106 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3) or
VI. a) a light chain variable region comprising the amino acid sequence of SEQ ID NO: 26 (L-CDR1); the amino acid sequence of SEQ ID NO: 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
VII. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 141 (H-CDR1); the amino acid sequence of SEQ ID NO: 62, 108, 109, 110, 111 or 142 (H-CDR2); the amino acid sequence of SEQ ID NO: 72 (H-CDR3).

According to certain embodiments, the anti-IL-36R antibody or antigen-binding fragment thereof comprises:
(i) a light chain variable region comprising the amino acid sequence of SEQ ID NO: 77; and a heavy chain variable region comprising the amino acid sequence of SEQ ID NO: 87; or
(ii) a light chain variable region comprising the amino acid sequence of SEQ ID NO: 77; and a heavy chain variable region comprising the amino acid sequence of SEQ ID NO: 88; or
(iii) a light chain variable region comprising the amino acid sequence of SEQ ID NO: 77; and a heavy chain variable region comprising the amino acid sequence of SEQ ID NO: 89; or
(iv) a light chain variable region comprising the amino acid sequence of SEQ ID NO: 80; and a heavy chain variable region comprising the amino acid sequence of SEQ ID NO: 87; or
(v) a light chain variable region comprising the amino acid sequence of SEQ ID NO: 80; and a heavy chain variable region comprising the amino acid sequence of SEQ ID NO: 88; or
(vi) a light chain variable region comprising the amino acid sequence of SEQ ID NO: 80; and a heavy chain variable region comprising the amino acid sequence of SEQ ID NO: 89; or
(vii) a light chain variable region comprising the amino acid sequence of SEQ ID NO: 85; and a heavy chain variable region comprising the amino acid sequence of SEQ ID NO: 100; or
(viii) a light chain variable region comprising the amino acid sequence of SEQ ID NO: 85; and a heavy chain variable region comprising the amino acid sequence of SEQ ID NO:101; or
(ix) a light chain variable region comprising the amino acid sequence of SEQ ID NO: 86; and a heavy chain variable region comprising the amino acid sequence of SEQ ID NO: 100; or
(x) a light chain variable region comprising the amino acid sequence of SEQ ID NO: 86; and a heavy chain variable region comprising the amino acid sequence of SEQ ID NO:101.

According to certain embodiments, the anti-IL-36R antibody or antigen-binding fragment thereof comprises:
i. a light chain comprising the amino acid sequence of SEQ ID NO: 115; and a heavy chain comprising the amino acid sequence of SEQ ID NO: 125; or
ii. a light chain comprising the amino acid sequence of SEQ ID NO: 115; and a heavy chain comprising the amino acid sequence of SEQ ID NO: 126; or
iii. a light chain comprising the amino acid sequence of SEQ ID NO: 115; and a heavy chain comprising the amino acid sequence of SEQ ID NO: 127; or
iv. a light chain comprising the amino acid sequence of SEQ ID NO: 118; and a heavy chain comprising the amino acid sequence of SEQ ID NO: 125; or
v. a light chain comprising the amino acid sequence of SEQ ID NO: 118; and a heavy chain comprising the amino acid sequence of SEQ ID NO: 126; or
vi. a light chain comprising the amino acid sequence of SEQ ID NO: 118; and a heavy chain comprising the amino acid sequence of SEQ ID NO: 127; or
vii. a light chain comprising the amino acid sequence of SEQ ID NO: 123; and a heavy chain comprising the amino acid sequence of SEQ ID NO: 138; or
viii. a light chain comprising the amino acid sequence of SEQ ID NO: 123; and a heavy chain comprising the amino acid sequence of SEQ ID NO: 139; or
ix. a light chain comprising the amino acid sequence of SEQ ID NO: 124; and a heavy chain comprising the amino acid sequence of SEQ ID NO: 138.

In one aspect, described and disclosed herein are anti-IL-36R antibodies, in particular humanized anti-IL-36R antibodies, and compositions and articles of manufacture comprising one or more anti-IL-36R antibody, in particular one or more humanized anti-IL-36R antibody of the present invention. Also described are binding agents that include an antigen-binding fragment of an anti-IL-36 antibody, in particular a humanized anti-IL-36R antibody.

Variable regions and CDRs of representative antibodies of the present invention are disclosed below:

L-CDR1 Amino Acid Sequences
>81B4vK32_3 L-CDR1
(SEQ ID NO: 26)
TASSSVSSSYFH
>81B4vK32_105 L-CDR1
(SEQ ID NO: 26)
TASSSVSSSYFH
>81B4vK32_116 L-CDR1
(SEQ ID NO: 26)
TASSSVSSSYFH
>81B4vK32_127 L-CDR1
(SEQ ID NO: 26)
TASSSVSSSYFH
>81B4vK32_138 L-CDR1
(SEQ ID NO: 26)
TASSSVSSSYFH
>81B4vK32_140 L-CDR1
(SEQ ID NO: 26)
TASSSVSSSYFH
>81B4vK32_141 L-CDR1
(SEQ ID NO: 26)
TASSSVSSSYFH
>81B4vK32_147 L-CDR1
(SEQ ID NO: 26)
TASSSVSSSYFH
>73C5vK39_2 L-CDR1
(SEQ ID NO: 27)
KASQDVGTNVL
>73C5vK39_7 L-CDR1
(SEQ ID NO: 27)
KASQDVGTNVL
>73C5vK39_15 L-CDR1
(SEQ ID NO: 27)
KASQDVGTNVL
L-CDR2 Amino Acid Sequences
>81B4vK32_3 L-CDR2
(SEQ ID NO: 102)
RTSTLAS
>81B4vK32_105 L-CDR2
(SEQ ID NO: 103)
RTSILAS
>81B4vK32_116 L-CDR2
(SEQ ID NO: 104)
RTSRLAS
>81B4vK32 _127 L-CDR2
(SEQ ID NO: 104)
RTSRLAS
>81B4vK32_138 L-CDR2
(SEQ ID NO: 104)
RTSRLAS
>81B4vK32_140 L-CDR2
(SEQ ID NO: 105)
RTSQLAS
>81B4vK32_141 L-CDR2
(SEQ ID NO: 106)
RTSKLAS
>81B4vK32_147 L-CDR2
(SEQ ID NO: 140)
RTSHLAS
>73C5vK39_2 L-CDR2
(SEQ ID NO: 36)
SASYRHS
>73C5vK39_7 L-CDR2
(SEQ ID NO: 36)
SASYRHS
>73C5vK39_15 L-CDR2
(SEQ ID NO: 36)
SASYRHS
L-CDR3 Amino Acid Sequences
>81B4vK32_3 L-CDR3
(SEQ ID NO: 44)
HQFHRSPLT
>81B4vK32_105 L-CDR3
(SEQ ID NO: 44)
HQFHRSPLT
>81B4vK32_116 L-CDR3
(SEQ ID NO: 44)
HQFHRSPLT
>81B4vK32_127 L-CDR3
(SEQ ID NO: 44)
HQFHRSPLT
>81B4vK32_138 L-CDR3
(SEQ ID NO: 44)
HQFHRSPLT
>81B4vK32_140 L-CDR3
(SEQ ID NO: 44)
HQFHRSPLT
>81B4vK32_141 L-CDR3
(SEQ ID NO: 44)
HQFHRSPLT
>81B4vK32_147 L-CDR3
(SEQ ID NO: 44)
HQFHRSPLT
>73C5vK39_2 L-CDR3
(SEQ ID NO: 45)
QQYSRYPLT
>73C5vK39_7 L-CDR3
(SEQ ID NO: 45)
QQYSRYPLT
>73C5vK39_15 L-CDR3
(SEQ ID NO: 45)
QQYSRYPLT
H-CDR1 Amino Acid Sequences
>81B4vH33_49 H-CDR1
(SEQ ID NO: 53)
GYSFTSSWIH
>81B4vH33_85T H-CDR1
(SEQ ID NO: 53)
GYSFTSSWIH
>81B4vH33_90 H-CDR1
(SEQ ID NO: 53)
GYSFTSSWIH
>81B4vH33_93 H-CDR1
(SEQ ID NO: 53)
GYSFTSSWIH
>81B4vH50_22 H-CDR1
(SEQ ID NO: 53)
GYSFTSSWIH
>81B4vH50_30 H-CDR1
(SEQ ID NO: 53)
GYSFTSSWIH
>81B4vH51_13 H-CDR1
(SEQ ID NO: 53)
GYSFTSSWIH
>81B4vH51_15 H-CDR1
(SEQ ID NO: 53)
GYSFTSSWIH
>81B4vH52_83 H-CDR1
(SEQ ID NO: 53)
GYSFTSSWIH
>73C5vH46_4 H-CDR1
(SEQ ID NO: 107)
GFSLTDYAVH
>73C5vH46_19 H-CDR1
(SEQ ID NO: 107)
GFSLTDYAVH
>73C5vH46_40 H-CDR1
(SEQ ID NO: 107)
GFSLTDYAVH
>73C5vH47_65 H-CDR1
(SEQ ID NO: 107)
GFSLTDYAVH
>73C5vH47_77 H-CDR1
(SEQ ID NO: 107)
GFSLTDYAVH
>73C5vH58_91 H-CDR1
(SEQ ID NO: 107)
GFSLTDYAVH
H-CDR2 Amino Acid Sequences
>81B4vH33_49 H-CDR2
(SEQ ID NO: 62)
EINPGNVRTNYNENF
>81B4vH33_85T H-CDR2
(SEQ ID NO: 62)
EINPGNVRTNYNENF
>81B4vH33_90 H-CDR2
(SEQ ID NO: 62)
EINPGNVRTNYNENF
>81B4vH33_93 H-CDR2
(SEQ ID NO: 62)
EINPGNVRTNYNENF
>81B4vH50_22 H-CDR2
(SEQ ID NO: 108)
EILPGVVRTNYNENF
>81B4vH50_30 H-CDR2
(SEQ ID NO: 109)
EINPGAVRTNYNENF
>81B4vH51_13 H-CDR2
(SEQ ID NO: 110)
EINPGLVRTNYNENF
>81B4vH51_15 H-CDR2
(SEQ ID NO: 109)
EINPGAVRTNYNENF
>81B4vH52_83 H-CDR2
(SEQ ID NO: 111)
EINPGSVRTNYNENF
>73C5vH46_4 H-CDR2
(SEQ ID NO: 64)
VIWSDGSTDYNAPFKS
>73C5vH46_19 H-CDR2
(SEQ ID NO: 64)
VIWSDGSTDYNAPFKS
>73C5vH46_40 H-CDR2
(SEQ ID NO: 64)
VIWSDGSTDYNAPFKS
>73C5vH47_65 H-CDR2
(SEQ ID NO: 64)
VIWSDGSTDYNAPFKS
>73C5vH47_77 H-CDR2
(SEQ ID NO: 63)
VIWSDGSTDFNAPFKS
>73C5vH58_91 H-CDR2
(SEQ ID NO: 64)
VIWSDGSTDYNAPFKS
H-CDR3 Amino Acid Sequences
>81B4vH33_49 H-CDR3
(SEQ ID NO: 72)
VFYGEPYFPY
>81B4vH33_85T H-CDR3
(SEQ ID NO: 72)
VFYGEPYFPY
>81B4vH33_90 H-CDR3
(SEQ ID NO: 72)
VFYGEPYFPY
>81B4vH33_93 H-CDR3
(SEQ ID NO: 72)
VFYGEPYFPY
>81B4vH50_22 H-CDR3
(SEQ ID NO: 72)
VFYGEPYFPY
>81B4vH50_30 H-CDR3
(SEQ ID NO: 72)
VFYGEPYFPY
>81B4vH51_13 H-CDR3
(SEQ ID NO: 72)
VFYGEPYFPY
>81B4vH51_15 H-CDR3
(SEQ ID NO: 72)
VFYGEPYFPY
>81B4vH52_83 H-CDR3
(SEQ ID NO: 72)
VFYGEPYFPY
>73C5vH46_4 H-CDR3
(SEQ ID NO: 73)
KGGYSGSWFAY
>73C5vH46_19 H-CDR3
(SEQ ID NO: 73)
KGGYSGSWFAY
>73C5vH46_40 H-CDR3
(SEQ ID NO: 73)
KGGYSGSWFAY
>73C5vH47_65 H-CDR3
(SEQ ID NO: 73)
KGGYSGSWFAY
>73C5vH47_77 H-CDR3
(SEQ ID NO: 73)
KGGYSGSWFAY
>73C5vH58_91 H-CDR3
(SEQ ID NO: 73)
KGGYSGSWFAY

In one aspect, a variable region of the present invention is linked to a constant region. For example, a variable region of the present invention is linked to a constant region shown below to form a heavy chain or a light chain of an antibody.

Heavy Chain Constant region linked downstream of
a humanized variable heavy region:
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRV
EPKSCDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK (SEQ ID
NO: 112)
Light Chain Constant region linked downstream of
a humanized variable light region:
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC (SEQ ID NO: 113)

Representative light chain and heavy chain sequences of the present invention are shown below (humanized variable regions derived from antibodies 81 B4 and 73C5 linked to constant regions).

Light Chain Amino Acid Sequences
>81B4vK32_3 Light Chain
EIVLTQSPGTLSLSPGERATMSCTASSSVSSSYFHWYQQKPGQAPRLLIYRTSTLASGIPDRFSGSGSGT
DFTLTISRLEPEDAATYYCHQFHRSPLTFGQGTKLEIKRTVAAPSVFIFPPSDEQLKSGTASVVCLLNNFYP
REAKVQWKVDNALQSGNSQESVTEQDSKDSTYSLSSTLTLSKADYEKHKVYACEVTHQGLSSPVTKSFN
RGEC (SEQ ID NO: 114)
>81B4vK32_105 Light Chain
EIVLTQSPGTLSLSPGERATMSCTASSSVSSSYFHWYQQKPGQAPRLLIYRTSILASGVPDRFSGSGSGT
DFTLTISRLEPEDFATYYCHQFHRSPLTFGQGTKLEIKRTVAAPSVFIFPPSDEQLKSGTASVVCLLNNFYP
REAKVQWKVDNALQSGNSQESVTEQDSKDSTYSLSSTLTLSKADYEKHKVYACEVTHQGLSSPVTKSFN
RGEC (SEQ ID NO: 115)
>81B4vK32_116 Light Chain
EIVLTQSPGTLSLSPGERATMSCTASSSVSSSYFHWYQQKPGQAPRLWIYRTSRLASGVPDRFSGSGSG
TDFTLTISRLEPEDAATYYCHQFHRSPLTFGQGTKLEIKRTVAAPSVFIFPPSDEQLKSGTASVVCLLNNFY
PREAKVQWKVDNALQSGNSQESVTEQDSKDSTYSLSSTLTLSKADYEKHKVYACEVTHQGLSSPVTKSF
NRGEC (SEQ ID NO: 116)
>81B4vK32_127 Light Chain
EIVLTQSPGTLSLSPGERATMTCTASSSVSSSYFHWYQQKPGQAPRLLIYRTSRLASGVPDRFSGSGSG
TDFTLTISRLEPEDFAVYYCHQFHRSPLTFGQGTKLEIKRTVAAPSVFIFPPSDEQLKSGTASVVCLLNNFY
PREAKVQWKVDNALQSGNSQESVTEQDSKDSTYSLSSTLTLSKADYEKHKVYACEVTHQGLSSPVTKSF
NRGEC (SEQ ID NO: 117)
>81B4vK32_138 Light Chain
QIVLTQSPGTLSLSPGERATMTCTASSSVSSSYFHWYQQKPGQAPRLWIYRTSRLASGVPDRFSGSGSG
TDFTLTISRLEPEDAATYYCHQFHRSPLTFGAGTKLEIKRTVAAPSVFIFPPSDEQLKSGTASVVCLLNNFY
PREAKVQWKVDNALQSGNSQESVTEQDSKDSTYSLSSTLTLSKADYEKHKVYACEVTHQGLSSPVTKSF
NRGEC (SEQ ID NO: 118)
>81B4vK32_140 Light Chain
QIVLTQSPGTLSLSPGERVTMSCTASSSVSSSYFHWYQQKPGQAPRLLIYRTSQLASGIPDRFSGSGSGT
DFTLTISRLEPEDAATYYCHQFHRSPLTFGQGTKLEIKRTVAAPSVFIFPPSDEQLKSGTASVVCLLNNFYP
REAKVQWKVDNALQSGNSQESVTEQDSKDSTYSLSSTLTLSKADYEKHKVYACEVTHQGLSSPVTKSFN
RGEC (SEQ ID NO: 119)
>81B4vK32_141 Light Chain
QIVLTQSPGTLSLSPGERATMTCTASSSVSSSYFHWYQQKPGQAPRLLIYRTSKLASGVPDRFSGSGSG
TDFTLTISRLEPEDFATYYCHQFHRSPLTFGQGTKLEIKRTVAAPSVFIFPPSDEQLKSGTASVVCLLNNFY
PREAKVQWKVDNALQSGNSQESVTEQDSKDSTYSLSSTLTLSKADYEKHKVYACEVTHQGLSSPVTKSF
NRGEC (SEQ ID NO: 120)
>81B4vK32_147 Light Chain
EIVLTQSPGTLSLSPGERATMSCTASSSVSSSYFHWYQQKPGQAPRLLIYRTSHLASGIPGRFSGSGSGT
DFTLTISRLEPEDAAVYYCHQFHRSPLTFGQGTKLEIKRTVAAPSVFIFPPSDEQLKSGTASVVCLLNNFYP
REAKVQWKVDNALQSGNSQESVTEQDSKDSTYSLSSTLTLSKADYEKHKVYACEVTHQGLSSPVTKSFN
RGEC (SEQ ID NO: 121)
>73C5vK39_2 Light Chain
EIVMTQSPATLSVSPGVRATLSCKASQDVGTNVLWYQQKPGQAPRPLIYSASYRHSGIPDRFSGSGSGT
EFTLTISSLQSEDFAEYFCQQYSRYPLTFGQGTKLEIKRTVAAPSVFIFPPSDEQLKSGTASVVCLLNNFYP
REAKVQWKVDNALQSGNSQESVTEQDSKDSTYSLSSTLTLSKADYEKHKVYACEVTHQGLSSPVTKSFN
RGEC (SEQ ID NO: 122)
>73C5vK39_7 Light Chain
EIVMTQSPATLSVSPGVRATLSCKASQDVGTNVLWYQQKPGQAPRPLIYSASYRHSGIPDRFSGSGSGT
EFTLTISSLQSEDFAVYYCQQYSRYPLTFGQGTKLEIKRTVAAPSVFIFPPSDEQLKSGTASVVCLLNNFYP
REAKVQWKVDNALQSGNSQESVTEQDSKDSTYSLSSTLTLSKADYEKHKVYACEVTHQGLSSPVTKSFN
RGEC (SEQ ID NO: 123)
>73C5vK39_15 Light Chain
EIVMTQSPATLSVSPGVRATLSCKASQDVGTNVLWYQQKPGQAPRPLIYSASYRHSGIPARFSGSGSGT
EFTLTISSLQSEDFAEYYCQQYSRYPLTFGQGTKLEIKRTVAAPSVFIFPPSDEQLKSGTASVVCLLNNFYP
REAKVQWKVDNALQSGNSQESVTEQDSKDSTYSLSSTLTLSKADYEKHKVYACEVTHQGLSSPVTKSFN
RGEC (SEQ ID NO: 124)
Heavy Chain Amino Acid Sequences
>81B4vH33_49 Heavy Chain
QVQLVQSGAEVKKPGASVKVSCKASGYSFTSSWIHWVRQAPGQGLEWIGEINPGNVRTNYNENFRNKA
TMTVDTSISTAYMELSRLRSDDTAVYYCAVVFYGEPYFPYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKRVEPKSCDKTHTCPPCPAPEAAGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGK (SEQ ID NO: 125)
>81B4vH33_85T Heavy Chain
QVQLVQSGAEVKKPGASVKVSCKASGYSFTSSWIHWVRQRPGQGLEWIGElNPGNVRTNYNENFRNRV
TMTVDTSISTAYMELSRLRSDDTAVYYCTVVFYGEPYFPYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKRVEPKSCDKTHTCPPCPAPEAAGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGK (SEQ ID NO: 126)
>81B4vH33_90 Heavy Chain
QVQLVQSGAEVKKPGASVKVSCKASGYSFTSSWIHWVKQAPGQGLEWMGEINPGNVRTNYNENFRNK
VTMTVDTSISTAYMELSRLRSDDTAVYYCTVVFYGEPYFPYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHK
PSNTKVDKRVEPKSCDKTHTCPPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFLYSKLTVDK
SRWQQGNVFSCSVMHEALHNHYTQKSLSLSPGK (SEQ ID NO: 127)
>81B4vH33_93 Heavy Chain
QVQLVQSGAEVKKPGASVKVSCKASGYSFTSSWIHWVRQRPGQGLEWMGElNPGNVRTNYNENFRNR
ATLTRDTSISTAYMELSRLRSDDTAVYYCAVVFYGEPYFPYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHK
PSNTKVDKRVEPKSCDKTHTCPPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFLYSKLTVDK
SRWQQGNVFSCSVMHEALHNHYTQKSLSLSPGK (SEQ ID NO: 128)
>81B4vH50_22 Heavy Chain
QVQLVQSGAEVKKPGASVKVSCKASGYSFTSSWIHWVRQRPGQGLEWMGElLPGVVRTNYNENFRNK
VTMTVDTSISTAYMELSRLRSDDTAVYYCTVVFYGEPYFPYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHK
PSNTKVDKRVEPKSCDKTHTCPPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFLYSKLTVDK
SRWQQGNVFSCSVMHEALHNHYTQKSLSLSPGK (SEQ ID NO: 129)
>81B4vH50_30 Heavy Chain
QVQLVQSGAEVKKPGASVKVSCKASGYSFTSSWIHWVRQRPGQGLEWIGElNPGAVRTNYNENFRNRV
TMTVDTSISTAYMELSRLRSDDTAVYYCTVVFYGEPYFPYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKRVEPKSCDKTHTCPPCPAPEAAGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGK (SEQ ID NO: 130)
>81B4vH51_13 Heavy Chain
QVQLVQSGAEVKKPGASVKVSCKASGYSFTSSWIHWVRQAPGQGLEWIGEINPGLVRTNYNENFRNKV
TMTVDTSISTAYMELSRLRSDDTAVYYCAVVFYGEPYFPYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKRVEPKSCDKTHTCPPCPAPEAAGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGK (SEQ ID NO: 131)
>81B4vH51_15 Heavy Chain
QVQLVQSGAEVKKPGASVKVSCKASGYSFTSSWIHWVRQAPGQGLEWIGEINPGAVRTNYNENFRNKV
TMTVDTSISTAYMELSRLRSDDTAVYYCAVVFYGEPYFPYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKRVEPKSCDKTHTCPPCPAPEAAGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGK (SEQ ID NO: 132)
>81B4vH52_83 Heavy Chain
QVQLVQSGAEVKKPGASVKVSCKASGYSFTSSWIHWVRQAPGQGLEWIGEINPGSVRTNYNENFRNKA
TMTVDTSISTAYMELSRLRSDDTAVYYCAVVFYGEPYFPYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKRVEPKSCDKTHTCPPCPAPEAAGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGK (SEQ ID NO: 133)
>73C5vH46_4 Heavy Chain
QVQLQESGPGLVKPSETLSITCTVSGFSLTDYAVHWIRQPPGKGLEWIGVIWSDGSTDYNAPFKSRVTIN
KDTSKSQVSFKMSSVQAADTAVYYCARKGGYSGSWFAYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKRVEPKSCDKTHTCPPCPAPEAAGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGK (SEQ ID NO: 134)
>73C5vH46_19 Heavy Chain
QVQLQESGPGLVKPSETLSITCTVSGFSLTDYAVHWIRQPPGKGLEWIGVIWSDGSTDYNAPFKSRVTIS
KDTSKNQVSLKMNSLTTDDTAVYYCARKGGYSGSWFAYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKRVEPKSCDKTHTCPPCPAPEAAGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGK (SEQ ID NO: 135)
>73C5vH46_40 Heavy Chain
QVQLQESGPGLVKPSETLSITCTVSGFSLTDYAVHWIRQPPGKGLEWIGVIWSDGSTDYNAPFKSRVTIS
KDNSKSQVSLKMNSVTVADTAVYYCARKGGYSGSWFAYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKRVEPKSCDKTHTCPPCPAPEAAGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGK (SEQ ID NO: 136)
>73C5vH47_65 Heavy Chain
QVQLQESGPGLVKPSETLSITCTVSGFSLTDYAVHWVRQPPGKGLEWIGVIWSDGSTDYNAPFKSRVTIS
KDTSKNQVSFKLSSVTVDDTAVYYCARKGGYSGSWFAYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKRVEPKSCDKTHTCPPCPAPEAAGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGK (SEQ ID NO: 137)
>73C5vH47_77 Heavy Chain
QVQLQESGPGLVAPSETLSLTCTVSGFSLTDYAVHWIRQFPGKGLEWIGVIWSDGSTDFNAPFKSRVTIS
KDTSKNQVSFKLSSVTTDDTAVYYCARKGGYSGSWFAYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKRVEPKSCDKTHTCPPCPAPEAAGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGK (SEQ ID NO: 138)
>73C5vH58_91 Heavy Chain
QVQLQESGPGLVKPSETLSITCTVSGFSLTDYAVHWIRQPPGKGLEWIGVIWSDGSTDYNAPFKSRVTIS
KDNSKSQVSFKMSSVTADDTAVYYCARKGGYSGSWFAYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKRVEPKSCDKTHTCPPCPAPEAAGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGK (SEQ ID NO: 139)

The CDRs listed above are defined using the Chothia numbering system (Al-Lazikani et al., (1997) JMB 273, 927-948).

In one aspect, an antibody of the present invention comprises 3 light chain CDRs and 3 heavy chain CDRs, for example as set forth above.

In one aspect, an antibody of the present invention comprises a light chain and a heavy chain variable region as set forth above. In one aspect, a light chain variable region of the invention is fused to a light chain constant region, for example a kappa or lambda constant region. In one aspect, a heavy chain variable region of the invention is fused to a heavy chain constant region, for example IgA, IgD, IgE, IgG or IgM, in particular, IgG₁, IgG₂, IgG₃ or IgG₄.

The present invention provides an anti-IL-36R antibody comprising a light chain comprising the amino acid sequence of SEQ ID NO: 115; and a heavy chain comprising the amino acid sequence of SEQ ID NO: 125 (Antibody B1).

The present invention provides an anti-IL-36R antibody comprising a light chain comprising the amino acid sequence of SEQ ID NO: 115; and a heavy chain comprising the amino acid sequence of SEQ ID NO: 126 (Antibody B2).

The present invention provides an anti-IL-36R antibody comprising a light chain comprising the amino acid sequence of SEQ ID NO: 115; and a heavy chain comprising the amino acid sequence of SEQ ID NO: 127 (Antibody B3).

The present invention provides an anti-IL-36R antibody comprising a light chain comprising the amino acid sequence of SEQ ID NO: 118; and a heavy chain comprising the amino acid sequence of SEQ ID NO: 125 (Antibody B4).

The present invention provides an anti-IL-36R antibody comprising a light chain comprising the amino acid sequence of SEQ ID NO: 118; and a heavy chain comprising the amino acid sequence of SEQ ID NO: 126 (Antibody B5).

The present invention provides an anti-IL-36R antibody comprising a light chain comprising the amino acid sequence of SEQ ID NO: 118; and a heavy chain comprising the amino acid sequence of SEQ ID NO: 127 Antibody B6).

The present invention provides an anti-IL-36R antibody comprising a light chain comprising the amino acid sequence of SEQ ID NO: 123; and a heavy chain comprising the amino acid sequence of SEQ ID NO: 138 (Antibody C3).

The present invention provides an anti-IL-36R antibody comprising a light chain comprising the amino acid sequence of SEQ ID NO: 123; and a heavy chain comprising the amino acid sequence of SEQ ID NO: 139 (Antibody C2).

The present invention provides an anti-IL-36R antibody comprising a light chain comprising the amino acid sequence of SEQ ID NO: 124; and a heavy chain comprising the amino acid sequence of SEQ ID NO: 138 (Antibody C1)

Representative antibodies of the present invention are shown below.

TABLE A

| Anti- | | |
|---|---|---|
| body | Light Chain Sequences | Heavy Chain Sequences |
| B1 | EIVLTQSPGTLSLSPGERATMSCTASSSV | QVQLVQSGAEVKKPGASVKVSCKASGYSFTSSW |
| | SSSYFHWYQQKPGQAPRLLIYRTSILASG | IHWVRQAPGQGLEWIGEINPGNVRTNYNENFRN |
| | VPDRFSGSGSGTDFTLTISRLEPEDFATY | KATMTVDTSISTAYMELSRLRSDDTAVYYCAVVF |
| | YCHQFHRSPLTFGQGTKLEIKRTVAAPS | YGEPYFPYWGQGTLVTVSSASTKGPSVFPLAPS |
| | VFIFPPSDEQLKSGTASVVCLLNNFYPRE | SKSTSGGTAALGCLVKDYFPEPVTVSWNSGALT |
| | AKVQWKVDNALQSGNSQESVTEQDSKD | SGVHTFPAVLQSSGLYSLSSVVTVPSSSLGTQTYI |
| | STYSLSSTLTLSKADYEKHKVYACEVTHQ | CNVNHKPSNTKVDKRVEPKSCDKTHTCPPCPAP |
| | GLSSPVTKSFNRGEC (SEQ ID NO: 115) | EAAGGPSVFLFPPKPKDTLMISRTPEVTCVVVDV |
| | | SHEDPEVKFNWYVDGVEVHNAKTKPREEQYNST |
| | | YRVVSVLTVLHQDWLNGKEYKCKVSNKALPAPIE |
| | | KTISKAKGQPREPQVYTLPPSREEMTKNQVSLTC |
| | | LVKGFYPSDIAVEWESNGQPENNYKTTPPVLDSD |
| | | GSFFLYSKLTVDKSRWQQGNVFSCSVMHEALHN |
| | | HYTQKSLSLSPGK (SEQ ID NO: 125) |
| B2 | EIVLTQSPGTLSLSPGERATMSCTASSSV | QVQLVQSGAEVKKPGASVKVSCKASGYSFTSSW |
| | SSSYFHWYQQKPGQAPRLLIYRTSILASG | IHWVRQRPGQGLEWIGEINPGNVRTNYNENFRN |
| | VPDRFSGSGSGTDFTLTISRLEPEDFATY | RVTMTVDTSISTAYMELSRLRSDDTAVYYCTVVF |
| | YCHQFHRSPLTFGQGTKLEIKRTVAAPS | YGEPYFPYWGQGTLVTVSSASTKGPSVFPLAPS |
| | VFIFPPSDEQLKSGTASVVCLLNNFYPRE | SKSTSGGTAALGCLVKDYFPEPVTVSWNSGALT |
| | AKVQWKVDNALQSGNSQESVTEQDSKD | SGVHTFPAVLQSSGLYSLSSVVTVPSSSLGTQTYI |
| | STYSLSSTLTLSKADYEKHKVYACEVTHQ | CNVNHKPSNTKVDKRVEPKSCDKTHTCPPCPAP |
| | GLSSPVTKSFNRGEC (SEQ ID NO: 115) | EAAGGPSVFLFPPKPKDTLMISRTPEVTCVVVDV |
| | | SHEDPEVKFNWYVDGVEVHNAKTKPREEQYNST |
| | | YRVVSVLTVLHQDWLNGKEYKCKVSNKALPAPIE |
| | | KTISKAKGQPREPQVYTLPPSREEMTKNQVSLTC |
| | | LVKGFYPSDIAVEWESNGQPENNYKTTPPVLDSD |
| | | GSFFLYSKLTVDKSRWQQGNVFSCSVMHEALHN |
| | | HYTQKSLSLSPGK (SEQ ID NO: 126) |
| B3 | EIVLTQSPGTLSLSPGERATMSCTASSSV | QVQLVQSGAEVKKPGASVKVSCKASGYSFTSSW |
| | SSSYFHWYQQKPGQAPRLLIYRTSILASG | IHWVKQAPGQGLEWMGEINPGNVRTNYNENFR |
| | VPDRFSGSGSGTDFTLTISRLEPEDFATY | NKVTMTVDTSISTAYMELSRLRSDDTAVYYCTVV |
| | YCHQFHRSPLTFGQGTKLEIKRTVAAPS | FYGEPYFPYWGQGTLVTVSSASTKGPSVFPLAP |
| | VFIFPPSDEQLKSGTASVVCLLNNFYPRE | SSKSTSGGTAALGCLVKDYFPEPVTVSWNSGAL |
| | AKVQWKVDNALQSGNSQESVTEQDSKD | TSGVHTFPAVLQSSGLYSLSSVVTVPSSSLGTQT |
| | STYSLSSTLTLSKADYEKHKVYACEVTHQ | YICNVNHKPSNTKVDKRVEPKSCDKTHTCPPCPA |
| | GLSSPVTKSFNRGEC (SEQ ID NO: 115) | PEAAGGPSVFLFPPKPKDTLMISRTPEVTCVVVD |
| | | VSHEDPEVKFNWYVDGVEVHNAKTKPREEQYNS |
| | | TYRVVSVLTVLHQDWLNGKEYKCKVSNKALPAPI |
| | | EKTISKAKGQPREPQVYTLPPSREEMTKNQVSLT |
| | | CLVKGFYPSDIAVEWESNGQPENNYKTTPPVLDS |
| | | DGSFFLYSKLTVDKSRWQQGNVFSCSVMHEALH |
| | | NHYTQKSLSLSPGK (SEQ ID NO: 127) |
| B4 | QIVLTQSPGTLSLSPGERATMTCTASSSV | QVQLVQSGAEVKKPGASVKVSCKASGYSFTSSW |
| | SSSYFHWYQQKPGQAPRLWIYRTSRLA | IHWVRQAPGQGLEWIGEINPGNVRTNYNENFRN |
| | SGVPDRFSGSGSGTDFTLTISRLEPEDA | KATMTVDTSISTAYMELSRLRSDDTAVYYCAVVF |
| | ATYYCHQFHRSPLTFGAGTKLEIKRTVAA | YGEPYFPYWGQGTLVTVSSASTKGPSVFPLAPS |
| | PSVFIFPPSDEQLKSGTASVVCLLNNFYP | SKSTSGGTAALGCLVKDYFPEPVTVSWNSGALT |
| | REAKVQWKVDNALQSGNSQESVTEQDS | SGVHTFPAVLQSSGLYSLSSVVTVPSSSLGTQTYI |
| | KDSTYSLSSTLTLSKADYEKHKVYACEVT | CNVNHKPSNTKVDKRVEPKSCDKTHTCPPCPAP |
| | HQGLSSPVTKSFNRGEC (SEQ ID NO: | EAAGGPSVFLFPPKPKDTLMISRTPEVTCVVVDV |
| | 118) | SHEDPEVKFNWYVDGVEVHNAKTKPREEQYNST |
| | | YRVVSVLTVLHQDWLNGKEYKCKVSNKALPAPIE |
| | | KTISKAKGQPREPQVYTLPPSREEMTKNQVSLTC |
| | | LVKGFYPSDIAVEWESNGQPENNYKTTPPVLDSD |
| | | GSFFLYSKLTVDKSRWQQGNVFSCSVMHEALHN |
| | | HYTQKSLSLSPGK (SEQ ID NO: 125) |
| B5 | QIVLTQSPGTLSLSPGERATMTCTASSSV | QVQLVQSGAEVKKPGASVKVSCKASGYSFTSSW |
| | SSSYFHWYQQKPGQAPRLWIYRTSRLA | IHWVRQRPGQGLEWIGEINPGNVRTNYNENFRN |
| | SGVPDRFSGSGSGTDFTLTISRLEPEDA | RVTMTVDTSISTAYMELSRLRSDDTAVYYCTVVF |
| | ATYYCHQFHRSPLTFGAGTKLEIKRTVAA | YGEPYFPYWGQGTLVTVSSASTKGPSVFPLAPS |
| | PSVFIFPPSDEQLKSGTASVVCLLNNFYP | SKSTSGGTAALGCLVKDYFPEPVTVSWNSGALT |
| | REAKVQWKVDNALQSGNSQESVTEQDS | SGVHTFPAVLQSSGLYSLSSVVTVPSSSLGTQTYI |
| | KDSTYSLSSTLTLSKADYEKHKVYACEVT | CNVNHKPSNTKVDKRVEPKSCDKTHTCPPCPAP |
| | HQGLSSPVTKSFNRGEC (SEQ ID NO: | EAAGGPSVFLFPPKPKDTLMISRTPEVTCVVVDV |
| | 118) | SHEDPEVKFNWYVDGVEVHNAKTKPREEQYNST |
| | | YRVVSVLTVLHQDWLNGKEYKCKVSNKALPAPIE |
| | | KTISKAKGQPREPQVYTLPPSREEMTKNQVSLTC |
| | | LVKGFYPSDIAVEWESNGQPENNYKTTPPVLDSD |
| | | GSFFLYSKLTVDKSRWQQGNVFSCSVMHEALHN |
| | | HYTQKSLSLSPGK (SEQ ID NO: 126) |
| B6 | QIVLTQSPGTLSLSPGERATMTCTASSSV | QVQLVQSGAEVKKPGASVKVSCKASGYSFTSSW |
| | SSSYFHWYQQKPGQAPRLWIYRTSRLA | IHWVKQAPGQGLEWMGEINPGNVRTNYNENFR |
| | SGVPDRFSGSGSGTDFTLTISRLEPEDA | NKVTMTVDTSISTAYMELSRLRSDDTAVYYCTVV |
| | ATYYCHQFHRSPLTFGAGTKLEIKRTVAA | FYGEPYFPYWGQGTLVTVSSASTKGPSVFPLAP |
| | PSVFIFPPSDEQLKSGTASVVCLLNNFYP | SSKSTSGGTAALGCLVKDYFPEPVTVSWNSGAL |
| | REAKVQWKVDNALQSGNSQESVTEQDS | TSGVHTFPAVLQSSGLYSLSSVVTVPSSSLGTQT |
| | KDSTYSLSSTLTLSKADYEKHKVYACEVT | YICNVNHKPSNTKVDKRVEPKSCDKTHTCPPCPA |
| | HQGLSSPVTKSFNRGEC (SEQ ID NO: | PEAAGGPSVFLFPPKPKDTLMISRTPEVTCVVVD |
| | 118) | VSHEDPEVKFNWYVDGVEVHNAKTKPREEQYNS |
| | | TYRVVSVLTVLHQDWLNGKEYKCKVSNKALPAPI |
| | | EKTISKAKGQPREPQVYTLPPSREEMTKNQVSLT |
| | | CLVKGFYPSDIAVEWESNGQPENNYKTTPPVLDS |
| | | DGSFFLYSKLTVDKSRWQQGNVFSCSVMHEALH |
| | | NHYTQKSLSLSPGK (SEQ ID NO: 127) |

TABLE B

| Anti- | | |
|---|---|---|
| body | Light Chain Sequences | Heavy Chain Sequences |
| C1 | EIVMTQSPATLSVSPGVRATLSCKASQD | QVQLQESGPGLVAPSETLSLTCTVSGFSLTDYAV |
| | VGTNVLWYQQKPGQAPRPLIYSASYRHS | HWIRQFPGKGLEWIGVIWSDGSTDFNAPFKSRVT |
| | GIPARFSGSGSGTEFTLTISSLQSEDFAE | ISKDTSKNQVSFKLSSVTTDDTAVYYCARKGGYS |
| | YYCQQYSRYPLTFGQGTKLEIKRTVAAP | GSWFAYWGQGTLVTVSSASTKGPSVFPLAPSSK |
| | SVFIFPPSDEQLKSGTASVVCLLNNFYPR | STSGGTAALGCLVKDYFPEPVTVSWNSGALTSG |
| | EAKVQWKVDNALQSGNSQESVTEQDSK | VHTFPAVLQSSGLYSLSSVVTVPSSSLGTQTYICN |
| | DSTYSLSSTLTLSKADYEKHKVYACEVTH | VNHKPSNTKVDKRVEPKSCDKTHTCPPCPAPEA |
| | QGLSSPVTKSFNRGEC (SEQ ID NO: | AGGPSVFLFPPKPKDTLMISRTPEVTCVVVDVSH |
| | 124) | EDPEVKFNWYVDGVEVHNAKTKPREEQYNSTYR |
| | | VVSVLTVLHQDWLNGKEYKCKVSNKALPAPIEKTI |
| | | SKAKGQPREPQVYTLPPSREEMTKNQVSLTCLV |
| | | KGFYPSDIAVEWESNGQPENNYKTTPPVLDSDG |
| | | SFFLYSKLTVDKSRWQQGNVFSCSVMHEALHNH |
| | | YTQKSLSLSPGK (SEQ ID NO: 138) |
| C2 | EIVMTQSPATLSVSPGVRATLSCKASQD | QVQLQESGPGLVKPSETLSITCTVSGFSLTDYAV |
| | VGTNVLWYQQKPGQAPRPLIYSASYRHS | HWIRQPPGKGLEWIGVIWSDGSTDYNAPFKSRV |
| | GIPDRFSGSGSGTEFTLTISSLQSEDFAV | TISKDNSKSQVSFKMSSVTADDTAVYYCARKGG |
| | YYCQQYSRYPLTFGQGTKLEIKRTVAAP | YSGSWFAYWGQGTLVTVSSASTKGPSVFPLAPS |
| | SVFIFPPSDEQLKSGTASVVCLLNNFYPR | SKSTSGGTAALGCLVKDYFPEPVTVSWNSGALT |
| | EAKVQWKVDNALQSGNSQESVTEQDSK | SGVHTFPAVLQSSGLYSLSSVVTVPSSSLGTQTYI |
| | DSTYSLSSTLTLSKADYEKHKVYACEVTH | CNVNHKPSNTKVDKRVEPKSCDKTHTCPPCPAP |
| | QGLSSPVTKSFNRGEC (SEQ ID NO: | EAAGGPSVFLFPPKPKDTLMISRTPEVTCVVVDV |
| | 123) | SHEDPEVKFNWYVDGVEVHNAKTKPREEQYNST |
| | | YRVVSVLTVLHQDWLNGKEYKCKVSNKALPAPIE |
| | | KTISKAKGQPREPQVYTLPPSREEMTKNQVSLTC |
| | | LVKGFYPSDIAVEWESNGQPENNYKTTPPVLDSD |
| | | GSFFLYSKLTVDKSRWQQGNVFSCSVMHEALHN |
| | | HYTQKSLSLSPGK (SEQ ID NO: 139) |
| C3 | EIVMTQSPATLSVSPGVRATLSCKASQD | QVQLQESGPGLVAPSETLSLTCTVSGFSLTDYAV |
| | VGTNVLWYQQKPGQAPRPLIYSASYRHS | HWIRQFPGKGLEWIGVIWSDGSTDFNAPFKSRVT |
| | GIPDRFSGSGSGTEFTLTISSLQSEDFAV | ISKDTSKNQVSFKLSSVTTDDTAVYYCARKGGYS |
| | YYCQQYSRYPLTFGQGTKLEIKRTVAAP | GSWFAYWGQGTLVTVSSASTKGPSVFPLAPSSK |
| | SVFIFPPSDEQLKSGTASVVCLLNNFYPR | STSGGTAALGCLVKDYFPEPVTVSWNSGALTSG |
| | EAKVQWKVDNALQSGNSQESVTEQDSK | VHTFPAVLQSSGLYSLSSVVTVPSSSLGTQTYICN |
| | DSTYSLSSTLTLSKADYEKHKVYACEVTH | VNHKPSNTKVDKRVEPKSCDKTHTCPPCPAPEA |
| | QGLSSPVTKSFNRGEC (SEQ ID NO: | AGGPSVFLFPPKPKDTLMISRTPEVTCVVVDVSH |
| | 123) | EDPEVKFNWYVDGVEVHNAKTKPREEQYNSTYR |
| | | VVSVLTVLHQDWLNGKEYKCKVSNKALPAPIEKTI |
| | | SKAKGQPREPQVYTLPPSREEMTKNQVSLTCLV |
| | | KGFYPSDIAVEWESNGQPENNYKTTPPVLDSDG |
| | | SFFLYSKLTVDKSRWQQGNVFSCSVMHEALHNH |
| | | YTQKSLSLSPGK (SEQ ID NO: 138) |

### Pharmaceutical Doses and Administration

Anti-IL-36R antibodies of the present invention are typically administered to a patient as a pharmaceutical composition described herein.

In one aspect, the present invention relates to a method for treating, preventing or ameliorating atopic dermatitis (AtD) in a subject, comprising administering to the subject a therapeutically effective amount of an anti-IL-36R antibody or an antigen-binding fragment thereof.

In one aspect, the present invention relates to a method of reducing microbial colonization of the skin in a subject with atopic dermatitis comprising administering to the subject a therapeutically effective amount of an anti-IL-36-R antibody or an antigen-binding fragment thereof. In an embodiment relating to this aspect, for example, the S. aureus colonization is reduced by at least 10% or by at least 20% from the baseline following the administration of the anti-IL-36-R antibody or an antigen-binding fragment thereof. In another embodiment relating to this aspect, the anti-IL-36R antibody is administered according to any of the doses and dosage regimens provided in Tables 1 and 2.

In one aspect, the present invention relates to a method of reducing susceptibility to a skin infection in a subject with atopic dermatitis comprising administering to the subject a therapeutically effective amount of an anti-IL-36-R antibody or an antigen-binding fragment thereof. In another embodiment relating to this aspect, the anti-IL-36R antibody is administered according to any of the doses and dosage regimens provided in Tables 1 and 2.

In one aspect, the present invention relates to a method of treating a skin disorder associated with AtD in a patient, said method(s) including administering or having administered to the patient a therapeutically effective amount of an anti-IL-36R antibody of the present invention. In another embodiment relating to this aspect, the anti-IL-36R antibody is administered according to any of the doses and dosage regimens provided in Tables 1 and 2.

In one aspect, the present invention relates to a method of treating skin inflammation associated with AtD in a subject, said method including administering or having administered to the subject a therapeutically effective amount of an anti-IL-36R antibody of the present invention or an antigen binding fragment thereof. In another embodiment relating to this aspect, the anti-IL-36R antibody is administered according to any of the doses and dosage regimens provided in Tables 1 and 2.

In one embodiment related to any of the above aspects, the anti-IL-36R antibody includes: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 53 or 141 (H-CDR1); the amino acid sequence of SEQ ID NO: 62, 108, 109, 110, 111 or 142 (H-CDR2); the amino acid sequence of SEQ ID NO: 72 (H-CDR3).

In one embodiment related to any of the above aspects, the anti-IL-36R antibody includes:

I. a) a light chain variable region comprising the amino acid sequence of SEQ ID NO: 26 (L-CDR1); the amino acid sequence of SEQ ID NO: 102 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or

II. a) a light chain variable region comprising the amino acid sequence of SEQ ID NO: 26 (L-CDR1); the amino acid sequence of SEQ ID NO: 103 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or

III. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or

IV. a) a light chain variable region comprising the amino acid sequence of SEQ ID NO: 26 (L-CDR1); the amino acid sequence of SEQ ID NO: 105 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or

V. a) a light chain variable region comprising the amino acid sequence of SEQ ID NO: 26 (L-CDR1); the amino acid sequence of SEQ ID NO: 106 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or

VI. a) a light chain variable region comprising the amino acid sequence of SEQ ID NO: 26 (L-CDR1); the amino acid sequence of SEQ ID NO: 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or

VII. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 141 (H-CDR1); the amino acid sequence of SEQ ID NO: 62, 108, 109, 110, 111 or 142 (H-CDR2); the amino acid sequence of SEQ ID NO: 72 (H-CDR3).

In one embodiment related to any of the above aspects, the anti-IL-36R antibody includes:

(i) a light chain variable region comprising the amino acid sequence of SEQ ID NO: 77; and a heavy chain variable region comprising the amino acid sequence of SEQ ID NO: 87; or

(ii) a light chain variable region comprising the amino acid sequence of SEQ ID NO: 77; and a heavy chain variable region comprising the amino acid sequence of SEQ ID NO: 88; or

(iii) a light chain variable region comprising the amino acid sequence of SEQ ID NO: 77; and a heavy chain variable region comprising the amino acid sequence of SEQ ID NO: 89; or

(iv) a light chain variable region comprising the amino acid sequence of SEQ ID NO: 80; and a heavy chain variable region comprising the amino acid sequence of SEQ ID NO: 87; or

(v) a light chain variable region comprising the amino acid sequence of SEQ ID NO: 80; and a heavy chain variable region comprising the amino acid sequence of SEQ ID NO: 88; or

(vi) a light chain variable region comprising the amino acid sequence of SEQ ID NO: 80; and a heavy chain variable region comprising the amino acid sequence of SEQ ID NO: 89; or

(vii) a light chain variable region comprising the amino acid sequence of SEQ ID NO: 85; and a heavy chain variable region comprising the amino acid sequence of SEQ ID NO: 100; or

(viii) a light chain variable region comprising the amino acid sequence of SEQ ID NO: 85; and a heavy chain variable region comprising the amino acid sequence of SEQ ID NO:101; or

(ix) a light chain variable region comprising the amino acid sequence of SEQ ID NO: 86; and a heavy chain variable region comprising the amino acid sequence of SEQ ID NO: 100; or

(x) a light chain variable region comprising the amino acid sequence of SEQ ID NO: 86; and a heavy chain variable region comprising the amino acid sequence of SEQ ID NO:101.

In one embodiment related to any of aspects first to fifth, the anti-IL-36R antibody includes:

i. a light chain comprising the amino acid sequence of SEQ ID NO: 115; and a heavy chain comprising the amino acid sequence of SEQ ID NO: 125; or

ii. a light chain comprising the amino acid sequence of SEQ ID NO: 115; and a heavy chain comprising the amino acid sequence of SEQ ID NO: 126; or

iii. a light chain comprising the amino acid sequence of SEQ ID NO: 115; and a heavy chain comprising the amino acid sequence of SEQ ID NO: 127; or

iv. a light chain comprising the amino acid sequence of SEQ ID NO: 118; and a heavy chain comprising the amino acid sequence of SEQ ID NO: 125; or

v. a light chain comprising the amino acid sequence of SEQ ID NO: 118; and a heavy chain comprising the amino acid sequence of SEQ ID NO: 126; or

vi. a light chain comprising the amino acid sequence of SEQ ID NO: 118; and a heavy chain comprising the amino acid sequence of SEQ ID NO: 127; or

vii. a light chain comprising the amino acid sequence of SEQ ID NO: 123; and a heavy chain comprising the amino acid sequence of SEQ ID NO: 138; or

viii. a light chain comprising the amino acid sequence of SEQ ID NO: 123; and a heavy chain comprising the amino acid sequence of SEQ ID NO: 139; or

ix. a light chain comprising the amino acid sequence of SEQ ID NO: 124; and a heavy chain comprising the amino acid sequence of SEQ ID NO: 138.

In an embodiment relating to any of the aspects and embodiments described herein, the anti-IL-36R antibody is administered subcutaneously or intravenously or by both routes simultaneously or sequentially and in any order. In a related embodiment, the subcutaneous administration comprises administration of 300 mg or 600 mg of the anti-IL-36R antibody. In a related embodiment, the intravenous administration comprises administering 300 mg, 600 mg, 900 mg or 1200 mg of the anti-IL-36R antibody. In a related embodiment, the subcutaneous administration comprises administration of one or more doses of 300 mg or one or more doses of 600 mg each of the anti-IL-36R antibody once every week (qw), once every 2 weeks (q2w), once every 4 weeks (q4w), once every 6 weeks (q6w) or once every 8 weeks (q8w), or a combination thereof.

In another embodiment relating to any of the aspects and embodiments described herein, the anti-IL-36R antibody is administered subcutaneously or intravenously or by both routes simultaneously or sequentially and in any order. In a related embodiment, the subcutaneous administration comprises initial doses (e.g., a lead-in or an induction dose regime). In a related embodiment, the subcutaneous administration further comprises subsequent doses (e.g., maintenance dosage regimen). In a related embodiment, the initial doses includes: (a) one or more doses of 150 mg each of the anti-IL-36R antibody administered daily for 2 weeks; or (b) one or more doses of 300 mg each of the anti-IL-36R antibody administered daily for 2 weeks; or (c) one or more doses of 600 mg each of the anti-IL-36R antibody administered twice, three times or four times in 4 weeks or administered twice per week for 2 weeks, or administered twice per week for 3 weeks, or administered twice per week for 4 weeks; or (d) one dose of 900 mg or 1200 mg of the anti-IL-36R antibody administered once; or (e) two doses of 900 mg or 1200 mg each of the anti-IL-36R antibody administered twice in three weeks (e.g., in weeks 0 and 2); the subsequent dose includes: (a) one or more doses of 300 mg each of the anti-IL-36R antibody administered q2w, q4w, q6w or q8w; or (b) one or more doses of 600 mg each of the anti-IL-36R antibody administered q2w, q4w, q6w or q8w; and wherein the administration of the subsequent dose is between 2 to 4 weeks or 2 weeks or 4 weeks after the administration of the last initial dose. In an embodiment, the administration of the first subsequent dose is between 2 to 4 weeks or 2 weeks or 4 weeks after the administration of the initial dose if only one initial dose is administered.

In another embodiment relating to any of the aspects and embodiments described herein, the anti-IL-36R antibody is administered subcutaneously at initial SC doses of 150 mg or 300 mg each (administered daily for 2 weeks) or 600 mg each (administered two times, three times, or four times in four weeks or twice per week in two weeks, three weeks or four weeks) or 900 mg or 1200 mg (administered once) or 900 mg or 1200 mg each (administered two times in three weeks) followed by subsequent SC doses of 300 mg or 600 mg (administered q2w, q4w, q6w or q8w). In an embodiment, the first subsequent dose is administered two to four weeks or two weeks or four weeks after the last initial dose. In an embodiment, the administration of the first subsequent dose is between two to four weeks or two weeks or four weeks after the administration of the initial dose if only one initial dose is administered.

In another embodiment related to any of the above aspects, the anti-IL-36R antibody is administered subcutaneously in an initial dose and a subsequent dose. In a related embodiment, the initial doses are 150 mg, 300 mg, 600 mg, 900 mg or 1200 mg each. In a related embodiment, the initial doses of 150 mg or 300 mg each are administered per day (in consecutive days) for two weeks. In a related embodiment, the initial doses of 600 mg each are administered once per week for two weeks including weeks 0 and 1; weeks 0 and 2; weeks 0 and 3; or weeks 0 and 4. In a related embodiment, the initial doses of 600 mg each are administered once per week for three weeks including weeks 0, 1 and 2; weeks 0, 1 and 3; weeks 0, 1 and 4; weeks 0, 2 and 3; weeks 0, 2 and 4; or weeks 0, 3 and 4. In a related embodiment, the initial doses of 600 mg each are administered once per week for four weeks including weeks 0, 1, 2 and 3; weeks 0, 1, 2 and 4; weeks 0, 1, 3 and 4; or weeks 0, 2, 3 and 4. In a related embodiment, the initial doses of 600 mg each are administered twice per week for 2 weeks. In a related embodiment, the initial doses of 600 mg each are administered twice per week for 3 weeks. In a related embodiment, the initial doses of 600 mg each are administered twice per week for 4 weeks. In a related embodiment, the initial dose of 900 mg or 1200 mg is administered once. In a related embodiment, the initial doses of 900 mg or 1200 mg each are administered twice in three weeks (e.g., in weeks 0 and 2). In a related embodiment, the subsequent doses include 300 mg or 600 mg of the anti-IL-36R.

In a related embodiment, the subsequent doses are 300 mg or 600 mg each. In a related embodiment, the subsequent dose administration begins two to four weeks after the initial dose administration ends. In a related embodiment, the subsequent doses of 300 mg or 600 mg each are administered q2w (once every 2 weeks), q4w (once every 4 weeks), q6w (once every 6 weeks) or q8w (once every 8 weeks).

Representative examples of dosage regimens according to the present invention are disclosed in Tables 1 and 2 below.

TABLE 1
Doses and Dosage Regimens

| Treatment dose (mg) | Dose frequency |
|---|---|
| 300 (IV or SC) | qw |
| 300 (IV or SC) | q2w |
| 300 (IV or SC) | q4w |
| 300 (IV or SC) | q6w |
| 300 (IV or SC) | q8w |
| 600 (IV or SC) | qw |
| 600 (IV or SC) | q2w |
| 600 (IV or SC) | q4w |
| 600 (IV or SC) | q6w |
| 600 (IV or SC) | q8w |

TABLE 2
Doses and Dosage Regimens

| Initial dose | Frequency | Subsequent | Frequency |
|---|---|---|---|
| (e.g., lead-in | of | dose (eg., | of |
| or induction | Initial | maintenance) | subsequent |
| dose) (mg) | dose | dose (mg) | doses |
| 150 (SC) | Per day for 2 weeks | 300 (SC) | q2w |
| 300 (SC) | Per day for 2 weeks | 300 (SC) | q2w |
| 600 (SC) | At weeks 0 and 1 | 300 (SC) | q2w |
| 600 (SC) | At weeks 0 and 2 | 300 (SC) | q2w |
| 600 (SC) | At weeks 0 and 3 | 300 (SC) | q2w |
| 600 (SC) | At weeks 0 and 4 | 300 (SC) | q2w |
| 600 (SC) | At weeks 0, 1 and 2 | 300 (SC) | q2w |
| 600 (SC) | At weeks 0, 1 and 3 | 300 (SC) | q2w |
| 600 (SC) | At weeks 0, 1 and 4 | 300 (SC) | q2w |
| 600 (SC) | At weeks 0, 2 and 3 | 300 (SC) | q2w |
| 600 (SC) | At weeks 0, 2 and 4 | 300 (SC) | q2w |
| 600 (SC) | At weeks 0, 3 and 4 | 300 (SC) | q2w |
| 600 (SC) | At weeks 0, 1, 2 and 3 | 300 (SC) | q2w |
| 600 (SC) | At weeks 0, 1, 2 and 4 | 300 (SC) | q2w |
| 600 (SC) | At weeks 0, 1, 3 and 4 | 300 (SC) | q2w |
| 600 (SC) | At weeks 0, 2, 3 and 4 | 300 (SC) | q2w |
| 600 (SC) | Twice per week for 2 weeks | 300 (SC) | q2w |
| 600 (SC) | Twice per week for 3 weeks | 300 (SC) | q2w |
| 600 (SC) | Twice per week for 4 weeks | 300 (SC) | q2w |
| 150 (SC) | Per day for 2 weeks | 300 (SC) | q4w |
| 300 (SC) | Per day for 2 weeks | 300 (SC) | q4w |
| 600 (SC) | At weeks 0 and 1 | 300 (SC) | q4w |
| 600 (SC) | At weeks 0 and 2 | 300 (SC) | q4w |
| 600 (SC) | At weeks 0 and 3 | 300 (SC) | q4w |
| 600 (SC) | At weeks 0 and 4 | 300 (SC) | q4w |
| 600 (SC) | At weeks 0, 1 and 2 | 300 (SC) | q4w |
| 600 (SC) | At weeks 0, 1 and 3 | 300 (SC) | q4w |
| 600 (SC) | At weeks 0, 1 and 4 | 300 (SC) | q4w |
| 600 (SC) | At weeks 0, 2 and 3 | 300 (SC) | q4w |
| 600 (SC) | At weeks 0, 2 and 4 | 300 (SC) | q4w |
| 600 (SC) | At weeks 0, 3 and 4 | 300 (SC) | q4w |
| 600 (SC) | At weeks 0, 1, 2 and 3 | 300 (SC) | q4w |
| 600 (SC) | At weeks 0, 1, 2 and 4 | 300 (SC) | q4w |
| 600 (SC) | At weeks 0, 1, 3 and 4 | 300 (SC) | q4w |
| 600 (SC) | At weeks 0, 2, 3 and 4 | 300 (SC) | q4w |
| 600 (SC) | Twice per week for 2 weeks | 300 (SC) | q4w |
| 600 (SC) | Twice per week for 3 weeks | 300 (SC) | q4w |
| 600 (SC) | Twice per week for 4 weeks | 300 (SC) | q4w |
| 150 (SC) | Per day for 2 weeks | 300 (SC) | q6w |
| 300 (SC) | Per day for 2 weeks | 300 (SC) | q6w |
| 600 (SC) | At weeks 0 and 1 | 300 (SC) | q6w |
| 600 (SC) | At weeks 0 and 2 | 300 (SC) | q6w |
| 600 (SC) | At weeks 0 and 3 | 300 (SC) | q6w |
| 600 (SC) | At weeks 0 and 4 | 300 (SC) | q6w |
| 600 (SC) | At weeks 0, 1 and 2 | 300 (SC) | q6w |
| 600 (SC) | At weeks 0, 1 and 3 | 300 (SC) | q6w |
| 600 (SC) | At weeks 0, 1 and 4 | 300 (SC) | q6w |
| 600 (SC) | At weeks 0, 2 and 3 | 300 (SC) | q6w |
| 600 (SC) | At weeks 0, 2 and 4 | 300 (SC) | q6w |
| 600 (SC) | At weeks 0, 3 and 4 | 300 (SC) | q6w |
| 600 (SC) | At weeks 0, 1, 2 and 3 | 300 (SC) | q6w |
| 600 (SC) | At weeks 0, 1, 2 and 4 | 300 (SC) | q6w |
| 600 (SC) | At weeks 0, 1, 3 and 4 | 300 (SC) | q6w |
| 600 (SC) | At weeks 0, 2, 3 and 4 | 300 (SC) | q6w |
| 600 (SC) | Twice per week for 2 weeks | 300 (SC) | q6w |
| 600 (SC) | Twice per week for 3 weeks | 300 (SC) | q6w |
| 600 (SC) | Twice per week for 4 weeks | 300 (SC) | q6w |
| 150 (SC) | Per day for 2 weeks | 300 (SC) | q8w |
| 300 (SC) | Per day for 2 weeks | 300 (SC) | q8w |
| 600 (SC) | At weeks 0 and 1 | 300 (SC) | q8w |
| 600 (SC) | At weeks 0 and 2 | 300 (SC) | q8w |
| 600 (SC) | At weeks 0 and 3 | 300 (SC) | q8w |
| 600 (SC) | At weeks 0 and 4 | 300 (SC) | q8w |
| 600 (SC) | At weeks 0, 1 and 2 | 300 (SC) | q8w |
| 600 (SC) | At weeks 0, 1 and 3 | 300 (SC) | q8w |
| 600 (SC) | At weeks 0, 1 and 4 | 300 (SC) | q8w |
| 600 (SC) | At weeks 0, 2 and 3 | 300 (SC) | q8w |
| 600 (SC) | At weeks 0, 2 and 4 | 300 (SC) | q8w |
| 600 (SC) | At weeks 0, 3 and 4 | 300 (SC) | q8w |
| 600 (SC) | At weeks 0, 1, 2 and 3 | 300 (SC) | q8w |
| 600 (SC) | At weeks 0, 1, 2 and 4 | 300 (SC) | q8w |
| 600 (SC) | At weeks 0, 1, 3 and 4 | 300 (SC) | q8w |
| 600 (SC) | At weeks 0, 2, 3 and 4 | 300 (SC) | q8w |
| 600 (SC) | Twice per week for 2 weeks | 300 (SC) | q8w |
| 600 (SC) | Twice per week for 3 weeks | 300 (SC) | q8w |
| 600 (SC) | Twice per week for 4 weeks | 300 (SC) | q8w |
| 150 (SC) | Per day for 2 weeks | 600 (SC) | q2w |
| 300 (SC) | Per day for 2 weeks | 600 (SC) | q2w |
| 600 (SC) | At weeks 0 and 1 | 600 (SC) | q2w |
| 600 (SC) | At weeks 0 and 2 | 600 (SC) | q2w |
| 600 (SC) | At weeks 0 and 3 | 600 (SC) | q2w |
| 600 (SC) | At weeks 0 and 4 | 600 (SC) | q2w |
| 600 (SC) | At weeks 0, 1 and 2 | 600 (SC) | q2w |
| 600 (SC) | At weeks 0, 1 and 3 | 600 (SC) | q2w |
| 600 (SC) | At weeks 0, 1 and 4 | 600 (SC) | q2w |
| 600 (SC) | At weeks 0, 2 and 3 | 600 (SC) | q2w |
| 600 (SC) | At weeks 0, 2 and 4 | 600 (SC) | q2w |
| 600 (SC) | At weeks 0, 3 and 4 | 600 (SC) | q2w |
| 600 (SC) | At weeks 0, 1, 2 and 3 | 600 (SC) | q2w |
| 600 (SC) | At weeks 0, 1, 2 and 4 | 600 (SC) | q2w |
| 600 (SC) | At weeks 0, 1, 3 and 4 | 600 (SC) | q2w |
| 600 (SC) | At weeks 0, 2, 3 and 4 | 600 (SC) | q2w |
| 600 (SC) | Twice per week for 2 weeks | 600 (SC) | q2w |
| 600 (SC) | Twice per week for 3 weeks | 600 (SC) | q2w |
| 600 (SC) | Twice per week for 4 weeks | 600 (SC) | q2w |
| 150 (SC) | Per day for 2 weeks | 600 (SC) | q4w |
| 300 (SC) | Per day for 2 weeks | 600 (SC) | q4w |
| 600 (SC) | At weeks 0 and 1 | 600 (SC) | q4w |
| 600 (SC) | At weeks 0 and 2 | 600 (SC) | q4w |
| 600 (SC) | At weeks 0 and 3 | 600 (SC) | q4w |
| 600 (SC) | At weeks 0 and 4 | 600 (SC) | q4w |
| 600 (SC) | At weeks 0, 1 and 2 | 600 (SC) | q4w |
| 600 (SC) | At weeks 0, 1 and 3 | 600 (SC) | q4w |
| 600 (SC) | At weeks 0, 1 and 4 | 600 (SC) | q4w |
| 600 (SC) | At weeks 0, 2 and 3 | 600 (SC) | q4w |
| 600 (SC) | At weeks 0, 2 and 4 | 600 (SC) | q4w |
| 600 (SC) | At weeks 0, 3 and 4 | 600 (SC) | q4w |
| 600 (SC) | At weeks 0, 1, 2 and 3 | 600 (SC) | q4w |
| 600 (SC) | At weeks 0, 1, 2 and 4 | 600 (SC) | q4w |
| 600 (SC) | At weeks 0, 1, 3 and 4 | 600 (SC) | q4w |
| 600 (SC) | At weeks 0, 2, 3 and 4 | 600 (SC) | q4w |
| 600 (SC) | Twice per week for 2 weeks | 600 (SC) | q4w |
| 600 (SC) | Twice per week for 3 weeks | 600 (SC) | q4w |
| 600 (SC) | Twice per week for 4 weeks | 600 (SC) | q4w |
| 150 (SC) | Per day for 2 weeks | 600 (SC) | q6w |
| 300 (SC) | Per day for 2 weeks | 600 (SC) | q6w |
| 600 (SC) | At weeks 0 and 1 | 600 (SC) | q6w |
| 600 (SC) | At weeks 0 and 2 | 600 (SC) | q6w |
| 600 (SC) | At weeks 0 and 3 | 600 (SC) | q6w |
| 600 (SC) | At weeks 0 and 4 | 600 (SC) | q6w |
| 600 (SC) | At weeks 0, 1 and 2 | 600 (SC) | q6w |
| 600 (SC) | At weeks 0, 1 and 3 | 600 (SC) | q6w |
| 600 (SC) | At weeks 0, 1 and 4 | 600 (SC) | q6w |
| 600 (SC) | At weeks 0, 2 and 3 | 600 (SC) | q6w |
| 600 (SC) | At weeks 0, 2 and 4 | 600 (SC) | q6w |
| 600 (SC) | At weeks 0, 3 and 4 | 600 (SC) | q6w |
| 600 (SC) | At weeks 0, 1, 2 and 3 | 600 (SC) | q6w |
| 600 (SC) | At weeks 0, 1, 2 and 4 | 600 (SC) | q6w |
| 600 (SC) | At weeks 0, 1, 3 and 4 | 600 (SC) | q6w |
| 600 (SC) | At weeks 0, 2, 3 and 4 | 600 (SC) | q6w |
| 600 (SC) | Twice per week for 2 weeks | 600 (SC) | q6w |
| 600 (SC) | Twice per week for 3 weeks | 600 (SC) | q6w |
| 600 (SC) | Twice per week for 4 weeks | 600 (SC) | q6w |
| 150 (SC) | Per day for 2 weeks | 600 (SC) | q8w |
| 300 (SC) | Per day for 2 weeks | 600 (SC) | q8w |
| 600 (SC) | At weeks 0 and 1 | 600 (SC) | q8w |
| 600 (SC) | At weeks 0 and 2 | 600 (SC) | q8w |
| 600 (SC) | At weeks 0 and 3 | 600 (SC) | q8w |
| 600 (SC) | At weeks 0 and 4 | 600 (SC) | q8w |
| 600 (SC) | At weeks 0, 1 and 2 | 600 (SC) | q8w |
| 600 (SC) | At weeks 0, 1 and 3 | 600 (SC) | q8w |
| 600 (SC) | At weeks 0, 1 and 4 | 600 (SC) | q8w |
| 600 (SC) | At weeks 0, 2 and 3 | 600 (SC) | q8w |
| 600 (SC) | At weeks 0, 2 and 4 | 600 (SC) | q8w |
| 600 (SC) | At weeks 0, 3 and 4 | 600 (SC) | q8w |
| 600 (SC) | At weeks 0, 1, 2 and 3 | 600 (SC) | q8w |
| 600 (SC) | At weeks 0, 1, 2 and 4 | 600 (SC) | q8w |
| 600 (SC) | At weeks 0, 1, 3 and 4 | 600 (SC) | q8w |
| 600 (SC) | At weeks 0, 2, 3 and 4 | 600 (SC) | q8w |
| 600 (SC) | Twice per week for 2 weeks | 600 (SC) | q8w |
| 600 (SC) | Twice per week for 3 weeks | 600 (SC) | q8w |
| 600 (SC) | Twice per week for 4 weeks | 600 (SC) | q8w |
| 900 (SC) | At weeks 0 and 2 | 300 (SC) | q2w |
| 900 (SC) | At week 0 | 300 (SC) | q2w |
| 900 (SC) | At weeks 0 and 2 | 300 (SC) | q4w |
| 900 (SC) | At week 0 | 300 (SC) | q4w |
| 900 (SC) | At weeks 0 and 2 | 300 (SC) | q6w |
| 900 (SC) | At week 0 | 300 (SC) | q6w |
| 900 (SC) | At weeks 0 and 2 | 300 (SC) | q8w |
| 900 (SC) | At week 0 | 300 (SC) | q8w |
| 900 (SC) | At weeks 0 and 2 | 600 (SC) | q2w |
| 900 (SC) | At week 0 | 600 (SC) | q2w |
| 900 (SC) | At weeks 0 and 2 | 600 (SC) | q4w |
| 900 (SC) | At week 0 | 600 (SC) | q4w |
| 900 (SC) | At weeks 0 and 2 | 600 (SC) | q6w |
| 900 (SC) | At week 0 | 600 (SC) | q6w |
| 900 (SC) | At weeks 0 and 2 | 600 (SC) | q8w |
| 900 (SC) | At week 0 | 600 (SC) | q8w |
| 1200 (SC) | At weeks 0 and 2 | 300 (SC) | q2w |
| 1200 (SC) | At week 0 | 300 (SC) | q2w |
| 1200 (SC) | At weeks 0 and 2 | 300 (SC) | q4w |
| 1200 (SC) | At week 0 | 300 (SC) | q4w |
| 1200 (SC) | At weeks 0 and 2 | 300 (SC) | q6w |
| 1200 (SC) | At week 0 | 300 (SC) | q6w |
| 1200 (SC) | At weeks 0 and 2 | 300 (SC) | q8w |
| 1200 (SC) | At week 0 | 300 (SC) | q8w |
| 1200 (SC) | At weeks 0 and 2 | 600 (SC) | q2w |
| 1200 (SC) | At week 0 | 600 (SC) | q2w |
| 1200 (SC) | At weeks 0 and 2 | 600 (SC) | q4w |
| 1200 (SC) | At week 0 | 600 (SC) | q4w |
| 1200 (SC) | At weeks 0 and 2 | 600 (SC) | q6w |
| 1200 (SC) | At week 0 | 600 (SC) | q6w |
| 1200 (SC) | At weeks 0 and 2 | 600 (SC) | q8w |
| 1200 (SC) | At week 0 | 600 (SC) | q8w |

SC: subcutanous or subcutanously
IV: intravenous or intravenously

In an embodiment relating to any of the above aspects, during or after treatment with an anti-IL-36R antibody of the present invention, the mammal or the patient is evaluated for improvement over placebo or baseline as defined by:
i. at least 10% improvement in Eczema Area and Severity Index (EASI) score at 4 and/or 16 weeks;
ii. at least 10% improvement in proportion of patients who attain EASI 50 at 4 and/or 16 weeks;
iii. at least 10% improvement in proportion of patients who attain EASI 75 at 4 and/or 16 weeks;
iv. at least 10% improvement in SCORAD, Max Itch Intensity and DLQI;
v. at least 10% improvement in number of patients with drug related Adverse Events (AEs) up to week 44;
vi. at least 10% improvement in absolute and percentage change from baseline in Eczema Area and Severity Index (EASI) at week 4;
vii. at least 10% improvement in proportion of patients with a 50% improvement in Eczema Area and Severity Index (EASI)(EASI50) at weeks 4 and/or 16;
viii. at least 5% improvement in proportion of patients with a 75% improvement in Eczema Area and Severity Index (EASI)(EASI75) at week 4 and/or 16;
ix. at least 10% improvement in SCORing of Atopic Dermatitis (SCORAD) at week 4 and/or 16;
x. at least 5% improvement in proportion of patients achieving at least a 2-grade reduction to clear (0) or almost clear (1) in Investigator's Global Assessment (IGA) at week 4 and/or 16;
xi. at least 10 percentage point difference in percentage change from baseline in EASI score at week 16;
xii. at least 10 percentage point difference in EASI50 response rate at week 16;
xiii. at least 5 percentage point difference in EASI75 response rate at week 16;
xiv. at least 10 percentage point difference in percentage change from baseline in SCORAD, Max Itch Intensity and DLQI at week 16;
xv. at least 10 percentage point difference in percentage change from baseline in Eczema Area and Severity Index (EASI) at week 44; or
xvi. at least 5 percentage point difference in IGA rate at week 16.

In a related embodiment, proportion of patients with a response to the administration is higher or significantly higher as compared to patients on placebo for any of the end points recited.

In an embodiment relating to any of the above aspects, the anti-IL-36R antibody or an antigen binding fragment thereof (disclosed herein) is present in a stable pharmaceutical formulation for administration to subject according to any one of the aspects of the present invention.

In another embodiment, the formulation comprises a therapeutic amount of an anti-IL-36R antibody (disclosed herein) and
i) a pharmaceutically acceptable buffer; or
ii) a pharmaceutically acceptable tonicifying agent; or
iii) a pharmaceutically acceptable stabilizing agent; or
iv) a pharmaceutically acceptable salt; or
v) a pharmaceutically acceptable surfactant; or
vi) a pharmaceutically acceptable buffer and a pharmaceutically acceptable tonicifying agent; or
vii) a pharmaceutically acceptable buffer, a pharmaceutically acceptable tonicifying agent and a pharmaceutically acceptable stabilizing agent; or
viii) a pharmaceutically acceptable buffer, a pharmaceutically acceptable tonicifying agent, a pharmaceutically acceptable stabilizing agent and a pharmaceutically acceptable salt; or
ix) a pharmaceutically acceptable buffer, a pharmaceutically acceptable tonicifying agent, a pharmaceutically acceptable stabilizing agent, a pharmaceutically acceptable salt and a pharmaceutically acceptable surfactant;
each in pharmaceutically acceptable quantities and at a pharmaceutically acceptable pH.

In another embodiment, the anti-IL-36R antibody or antigen binding fragment thereof is present in the formulation at a concentration of about 15 mg/mL, about 20 mg/mL, about 25 mg/mL, about 30 mg/mL, about 60 mg/mL, about 75 mg/mL, about 80 mg/mL, about 100 mg/mL or about 150 mg/mL. In another related embodiment, the pharmaceutically acceptable buffer is present in the formulation at a concentration within the range from about 20 mM to about 80 mM, or at a concentration of about 20 mM, about 25 mM, about 35 mM, about 40 mM, about 45 mM, about 50 mM, about 60 mM. In another related embodiment, the pharmaceutically acceptable tonicifying agent is present in the formulation at a concentration within the range from about 100 mM to about 250 mM, or at a concentration of about 100 mM, about 120 mM, about 150 mM, about 180 mM, about 200 mM. In another related embodiment, the pharmaceutically acceptable stabilizing agent is present in the formulation at a concentration within the range from about 0 mM to about 80 mM, or at a concentration of about 25 mM or about 50 mM. In another related embodiment, the pharmaceutically acceptable salt is present in the formulation at a concentration of within the range from about 0 to about 150 mM, or at a concentration of about 3 mM, 5 mM, 10 mM, 25 mM or 50 mM. In another related embodiment, the pharmaceutically acceptable surfactant is present in the formulation at a concentration within the range from about 0 g/L to about 1.5 g/L, or at a concentration of about 0.1 g/L, 0.2 g/L, 0.4 g/L, 0.5 g/L or 1 g/L. In an embodiment related to the first aspect, the formulation is characterized by a pH within the range from about 5 to about 8. In another related embodiment, the pH is about 5, about 5.5, about 6, about 6.5, about 7, about 7.5 or about 8.

In another embodiment, the buffer comprises histidine, phosphate, succinate, citrate, acetate or TRIS; the tonicifying agent is one or more sugar and/or polyol including sucrose, trehalose, sorbitol, magnesium sulfate (MgSO₄), glycerol, mannitol or dextrose; the stabilizer comprises an amino acid including arginine, histidine, glycine, cysteine, proline, methionine, lysine, aspartate, glutamate or pharmaceutically acceptable salts thereof; the salt comprises sodium chloride (NaCl), magnesium chloride (MgCl2), potassium chloride (KCl), lithium chloride (LiCl), calcium chloride (CaCl2)), boric acid salts or zinc chloride (ZnCl2); and the surfactant comprises poloxamer 188, polysorbate 20, polysorbate 40, polysorbate 60 or polysorbate 80.

In one embodiment, the method of treatment according to any of the aspects described herein, includes administering to the subject a therapeutic amount of a stable pharmaceutical formulation comprising from about 20 mg/mL to about 150 mg/mL of an anti-IL-36R antibody, about 20 mM to about 80 mM of a pharmaceutically acceptable buffer (e.g., acetate buffer), about 100 mM to about 250 mM of a pharmaceutically acceptable tonicifying agent (e.g., sucrose), about 0 mM to about 80 mM of a pharmaceutically acceptable stabilizing agent (e.g., arginine) or a pharmaceutically acceptable salt thereof, about 0 to about 150 mM of a pharmaceutically acceptable salt (e.g., sodium chloride), and a pharmaceutically acceptable surfactant (e.g., polysorbate 20) in an amount about 0 g/L to about 1.5 g/L, wherein the atopic dermatitis (AtD) in the subject is treated, prevented or ameliorated, wherein the microbial colonization of the skin in the subject with atopic dermatitis is reduced or inhibited, wherein the susceptibility to a skin infection in the subject with atopic dermatitis is reduced or inhibited, wherein the skin disorder associated with AtD in the subject is treated or prevented, wherein the skin inflammation associated with AtD in the subject is treated. In a related embodiment, the stable pharmaceutical formulation is an aqueous pharmaceutical formulation. In a related embodiment, the pH of the aqueous pharmaceutical formulation is about 5 to about 7. In a related embodiment, the pharmaceutical formulation is for an intravenous administration to the subject. In a related embodiment, the pharmaceutical formulation is for a subcutaneous administration to the subject. In a related embodiment, the pharmaceutical formulation for the intravenous administration comprises an anti-IL-36R antibody in an amount of about 60 mg/mL. In a related embodiment, the pharmaceutical formulation for a subcutaneous administration comprises an anti-IL-36R antibody in an amount of about 150 mg/mL. In a related embodiment, the anti-IL-36R antibody comprising: (i) a light chain including an amino acid sequence set forth as SEQ ID NO:118 and a heavy chain including an amino acid sequence set forth as SEQ ID NO:125; or (ii) a light chain including an amino acid sequence set forth as SEQ ID NO:118 and a heavy chain including an amino acid sequence set forth as SEQ ID NO:126; or (iii) a light chain including an amino acid sequence set forth as SEQ ID NO:118 and a heavy chain including an amino acid sequence set forth as SEQ ID NO:127. In a related embodiment, the anti-IL-36R antibody comprising: a light chain variable region comprising the amino acid sequence of SEQ ID NO: 77; and a heavy chain variable region comprising the amino acid sequence of SEQ ID NO: 87; or a light chain variable region comprising the amino acid sequence of SEQ ID NO: 77; and a heavy chain variable region comprising the amino acid sequence of SEQ ID NO: 88; or a light chain variable region comprising the amino acid sequence of SEQ ID NO: 77; and a heavy chain variable region comprising the amino acid sequence of SEQ ID NO: 89; or a light chain variable region comprising the amino acid sequence of SEQ ID NO: 80; and a heavy chain variable region comprising the amino acid sequence of SEQ ID NO: 87; or a light chain variable region comprising the amino acid sequence of SEQ ID NO: 80; and a heavy chain variable region comprising the amino acid sequence of SEQ ID NO: 88; or a light chain variable region comprising the amino acid sequence of SEQ ID NO: 80; and a heavy chain variable region comprising the amino acid sequence of SEQ ID NO: 89.

In one embodiment, the method of treatment according to any of the preceding aspects, comprises administering to the subject a therapeutic amount of a stable pharmaceutical formulation selected from the group consisting of consisting of:
I. formulation including about 20 mg/mL to about 150 mg/mL of the anti-IL-36R antibody, about 40 mM histidine, about 120 mM sucrose, about 50 mM L-Arginine, about 5 mM NaCl and about 1.0 g/L Polysorbate 20, with a pH of about 6.0;
II. formulation including about 20 mg/mL to about 150 mg/mL of the anti-IL-36R antibody, about 45 mM acetate, about 150 mM sucrose, about 25 mM L-Arginine, about 0.4 g/L Polysorbate 20, with a pH of about 5.5;
III. formulation including about 20 mg/mL to about 150 mg/mL of the anti-IL-36R antibody, about 45 mM acetate, about 180 mM sucrose, about 25 mM Glycine, about 0.4 g/L Polysorbate 80, with a pH of about 5.5;
IV. formulation including about 20 mg/mL to about 150 mg/mL of the anti-IL-36R antibody, about 25 mM citrate, about 150 mM trehalose, about 25 mM methionine, about 0.2 g/L Polysorbate 20, with a pH of about 6.0;
V. formulation including about 20 mg/mL to about 150 mg/mL of the anti-IL-36R antibody, about 25 mM histidine, about 180 mM sucrose, about 20 mM mannitol, about 0.2 g/L Polysorbate 20, with a pH of about 6.5;
VI. formulation including about 20 mg/mL to about 150 mg/mL of the anti-IL-36R antibody, about 25 mM citrate, about 200 mM sucrose, about 0.4 g/L Polysorbate 80, with a pH of about 6.5;
VII. formulation including about 20 mg/mL to about 150 mg/mL of the anti-IL-36R antibody, about 45 mM acetate, about 150 mM sucrose, about 25 mM L-Arginine, about 0.4 g/L Polysorbate 20, with a pH of about 5.5;
VIII. formulation including about 20 mg/mL to about 150 mg/mL of the anti-IL-36R antibody, about 35 mM histidine, about 180 mM trehalose, about 25 mM L-Arginine, about 3 mM NaCl, about 0.4 g/L Polysorbate 80, with a pH of about 6.0;
IX. formulation including about 20 mg/mL to about 150 mg/mL of the anti-IL-36R antibody, about 25 mM acetate, about 100 mM mannitol, about 50 mM NaCl, about 0.2 g/L Polysorbate 20, with a pH of about 5.5;
X. formulation including about 20 mg/mL to about 150 mg/mL of the anti-IL-36R antibody, about 20 mM succinate, about 220 mM sucrose, about 0.1 g/L Polysorbate 80, with a pH of about 6.0; and
XI. formulation including about 20 mg/mL to about 150 mg/mL of the anti-IL-36R antibody, about 25 mM citrate, about 0.4 g/L Polysorbate 20, with a pH of about 6.5,
wherein the atopic dermatitis (AtD) in the subject is treated, prevented or ameliorated, wherein the microbial colonization of the skin in the subject with atopic dermatitis is reduced or inhibited, wherein the susceptibility to a skin infection in the subject with atopic dermatitis is reduced or inhibited, wherein the skin disorder associated with AtD in the subject is treated or prevented, wherein the skin inflammation associated with AtD in the subject is treated.

In a related embodiment, the stable pharmaceutical formulation is an aqueous pharmaceutical formulation. In a related embodiment, the pharmaceutical formulation is for an intravenous administration to the subject. In a related embodiment, the pharmaceutical formulation is for a subcutaneous administration to the subject. In a related embodiment, the pharmaceutical formulation for an intravenous administration comprises an anti-IL-36R antibody in an amount of about 60 mg/mL. In a related embodiment, the pharmaceutical formulation for a subcutaneous administration comprises an anti-IL-36R antibody in an amount of about 150 mg/mL. In a related embodiment, the anti-IL-36R antibody comprising: (i) a light chain including an amino acid sequence set forth as SEQ ID NO:118 and a heavy chain including an amino acid sequence set forth as SEQ ID NO:125; or (ii) a light chain including an amino acid sequence set forth as SEQ ID NO:118 and a heavy chain including an amino acid sequence set forth as SEQ ID NO:126; or (iii) a light chain including an amino acid sequence set forth as SEQ ID NO:118 and a heavy chain including an amino acid sequence set forth as SEQ ID NO:127. In a related embodiment, the anti-IL-36R antibody comprising: a light chain variable region comprising the amino acid sequence of SEQ ID NO: 77; and a heavy chain variable region comprising the amino acid sequence of SEQ ID NO: 87; or a light chain variable region comprising the amino acid sequence of SEQ ID NO: 77; and a heavy chain variable region comprising the amino acid sequence of SEQ ID NO: 88; or a light chain variable region comprising the amino acid sequence of SEQ ID NO: 77; and a heavy chain variable region comprising the amino acid sequence of SEQ ID NO: 89; or a light chain variable region comprising the amino acid sequence of SEQ ID NO: 80; and a heavy chain variable region comprising the amino acid sequence of SEQ ID NO: 87; or a light chain variable region comprising the amino acid sequence of SEQ ID NO: 80; and a heavy chain variable region comprising the amino acid sequence of SEQ ID NO: 88; or a light chain variable region comprising the amino acid sequence of SEQ ID NO: 80; and a heavy chain variable region comprising the amino acid sequence of SEQ ID NO: 89.

In one embodiment, the method of treatment according to any of the preceding aspects, comprises administering to the subject a therapeutic amount of a stable pharmaceutical formulation selected from the group consisting of:
I. formulation including about 20 mg/mL of the anti-IL-36R antibody, about 40 mM histidine, about 120 mM sucrose, about 50 mM L-Arginine, about 5 mM NaCl and about 1.0 g/L Polysorbate 20, with a pH of about 6.0;
II. formulation including about 60 mg/mL of the anti-IL-36R antibody, about 45 mM acetate, about 150 mM sucrose, about 25 mM L-Arginine, about 0.4 g/L Polysorbate 20, with a pH of about 5.5;
III. formulation including about 20 mg/mL of the anti-IL-36R antibody, about 45 mM acetate, about 180 mM sucrose, about 25 mM Glycine, about 0.4 g/L Polysorbate 80, with a pH of about 5.5;
IV. formulation including about 150 mg/mL of the anti-IL-36R antibody, about 25 mM citrate, about 150 mM trehalose, about 25 mM methionine, about 0.2 g/L Polysorbate 20, with a pH of about 6.0;
V. formulation including about 150 mg/mL of the anti-IL-36R antibody, about 25 mM histidine, about 180 mM sucrose, about 20 mM mannitol, about 0.2 g/L Polysorbate 20, with a pH of about 6.5;
VI. formulation including about 20 mg/mL of the anti-IL-36R antibody, about 25 mM citrate, about 200 mM sucrose, about 0.4 g/L Polysorbate 80, with a pH of about 6.5;
VII. formulation including about 150 mg/mL of the anti-IL-36R antibody, about 45 mM acetate, about 150 mM sucrose, about 25 mM L-Arginine, about 0.4 g/L Polysorbate 20, with a pH of about 5.5;
VIII. formulation including about 15 mg/mL of the anti-IL-36R antibody, about 35 mM histidine, about 180 mM trehalose, about 25 mM L-Arginine, about 3 mM NaCl, about 0.4 g/L Polysorbate 80, with a pH of about 6.0;
IX. formulation including about 80 mg/mL of the anti-IL-36R antibody, about 25 mM acetate, about 100 mM mannitol, about 50 mM NaCl, about 0.2 g/L Polysorbate 20, with a pH of about 5.5;
X. formulation including about 100 mg/mL of the anti-IL-36R antibody, about 20 mM succinate, about 220 mM sucrose, about 0.1 g/L Polysorbate 80, with a pH of about 6.0; and
XI. formulation including about 60 mg/mL of the anti-IL-36R antibody, about 25 mM citrate, about 0.4 g/L Polysorbate 20, with a pH of about 6.5,
wherein the atopic dermatitis (AtD) in the subject is treated, prevented or ameliorated, wherein the microbial colonization of the skin in the subject with atopic dermatitis is reduced or inhibited, wherein the susceptibility to a skin infection in the subject with atopic dermatitis is reduced or inhibited, wherein the skin disorder associated with AtD in the subject is treated or prevented, wherein the skin inflammation associated with AtD in the subject is treated.

In a related embodiment, the stable pharmaceutical formulation is an aqueous pharmaceutical formulation. In a related embodiment, the pharmaceutical formulation is for an intravenous administration to the subject. In a related embodiment, the pharmaceutical formulation is for a subcutaneous administration to the subject. In a related embodiment, the pharmaceutical formulation for an intravenous administration comprises an anti-IL-36R antibody in an amount of about 60 mg/mL. In a related embodiment, the pharmaceutical formulation for a subcutaneous administration comprises an anti-IL-36R antibody in an amount of about 150 mg/mL. In a related embodiment, the anti-IL-36R antibody comprising: (i) a light chain including an amino acid sequence set forth as SEQ ID NO:118 and a heavy chain including an amino acid sequence set forth as SEQ ID NO:125; or (ii) a light chain including an amino acid sequence set forth as SEQ ID NO:118 and a heavy chain including an amino acid sequence set forth as SEQ ID NO:126; or (iii) a light chain including an amino acid sequence set forth as SEQ ID NO:118 and a heavy chain including an amino acid sequence set forth as SEQ ID NO:127. In a related embodiment, the anti-IL-36R antibody comprising: a light chain variable region comprising the amino acid sequence of SEQ ID NO: 77; and a heavy chain variable region comprising the amino acid sequence of SEQ ID NO: 87; or a light chain variable region comprising the amino acid sequence of SEQ ID NO: 77; and a heavy chain variable region comprising the amino acid sequence of SEQ ID NO: 88; or a light chain variable region comprising the amino acid sequence of SEQ ID NO: 77; and a heavy chain variable region comprising the amino acid sequence of SEQ ID NO: 89; or a light chain variable region comprising the amino acid sequence of SEQ ID NO: 80; and a heavy chain variable region comprising the amino acid sequence of SEQ ID NO: 87; or a light chain variable region comprising the amino acid sequence of SEQ ID NO: 80; and a heavy chain variable region comprising the amino acid sequence of SEQ ID NO: 88; or a light chain variable region comprising the amino acid sequence of SEQ ID NO: 80; and a heavy chain variable region comprising the amino acid sequence of SEQ ID NO: 89.

In one embodiment, the present invention relates to a method of treating a skin disorder associated with AtD in a patient, said method(s) including administering or having administered to the patient a therapeutically effective amount of an anti-IL-36R antibody of the present invention subcutaneously. In a related embodiment, the subcutaneous administration comprises administration of one or more doses of 300 mg or one or more doses of 600 mg each of the anti-IL-36R antibody once every week(qw), once every 2 weeks (q2w), once every 4 weeks (q4w), once every 6 weeks (q6w) or once every 8 weeks (q8w), or a combination thereof.

In one embodiment, the present invention relates to a method of treating a skin disorder associated with AtD in a patient, said method(s) including administering or having administered to the patient a therapeutically effective amount of an anti-IL-36R antibody of the present invention. In a related embodiment, the anti-IL-36R antibody is administered subcutaneously in an initial dose and a subsequent dose. In a related embodiment, the initial doses are administered subcutaneously. In a related embodiment, the subsequent doses are administered subcutaneously. In a related embodiment, the initial doses includes: (a) one or more doses of 150 mg each of the anti-IL-36R antibody administered daily for 2 weeks; or (b) one or more doses of 300 mg each of the anti-IL-36R antibody administered daily for 2 weeks; or (c) one or more doses of 600 mg each of the anti-IL-36R antibody administered twice, three times or four times in 4 weeks or administered twice per week for 2 weeks, or administered twice per week for 3 weeks, or administered twice per week for 4 weeks; or (d) one dose of 900 mg or 1200 mg of the anti-IL-36R antibody administered once; or (e) two doses of 900 mg or 1200 mg each of the anti-IL-36R antibody administered twice in three weeks (e.g., in weeks 0 and 2); the subsequent dose includes: (a) one or more doses of 300 mg each of the anti-IL-36R antibody administered q2w, q4w, q6w or q8w; or (b) one or more doses of 600 mg each of the anti-IL-36R antibody administered q2w, q4w, q6w or q8w; and wherein the administration of the subsequent dose is between 2 to 4 weeks or 2 weeks or 4 weeks after the administration of the last initial dose. In an embodiment, the administration of the first subsequent dose is between 2 to 4 weeks or 2 weeks or 4 weeks after the administration of the initial dose if only one initial dose is administered.

In one embodiment related to any of the above aspects or their related embodiment(s), at least 10%, 20%, 30%, 40%, 50%, 60%, 70% or 80% of the patients treated with an anti-IL-36R antibody of the present invention experience at least 10%, at least 20%, at least 30%, at least 40%, or at least 50% improvement in EASI score at 4 and/or 16 weeks as compared to placebo or baseline.

In one embodiment related to any of the above aspects or their related embodiment(s), at least 10%, 20%, 30%, 40%, 50%, 60%, 70% or 80% of the patients treated with an anti-IL-36R antibody of the present invention attain EASI 50 at 4 and/or 16 weeks as compared to placebo group or their baseline.

In one embodiment related to any of the above aspects or their related embodiment(s), at least 10%, 20%, 30%, 40%, 50%, 60%, 70% or 80% of the patients treated with an anti-IL-36R antibody of the present invention attain EASI 75 at 4 and/or 16 weeks as compared to placebo group or their baseline.

In one embodiment related to any of the above aspects or their related embodiment(s), at least 10%, 20%, 30%, 40%, 50%, 60%, 70% or 80% of the patients treated with an anti-IL-36R antibody of the present invention experience at least 10%, at least 20%, at least 30%, at least 40%, or at least 50% improvement in SCORAD, Max Itch Intensity and DLQI.

In one embodiment related to any of the above aspects or their related embodiment(s), at least 10%, 20%, 30%, 40%, 50%, 60%, 70% or 80% of the patients treated with an anti-IL-36R antibody of the present invention experience less drug related Adverse Events (AEs) up to week 44 as compared to placebo group or their baseline.

In one embodiment related to any of the above aspects or their related embodiment(s), at least 10%, 20%, 30%, 40%, 50%, 60%, 70% or 80% of the patients treated with an anti-IL-36R antibody of the present invention experience at least 10%, at least 20%, at least 30%, at least 40%, or at least 50% improvement in absolute and percentage change from baseline in Eczema Area and Severity Index (EASI) at week 4 as compared to placebo group or their baseline.

In one embodiment related to any of the above aspects or their related embodiment(s), at least 10%, 20%, 30%, 40%, 50%, 60%, 70% or 80% of the patients treated with an anti-IL-36R antibody of the present invention achieve at least 10%, at least 20%, at least 30%, at least 40%, or at least 50% improvement in Eczema Area and Severity Index (EASI)(EASI50) at weeks 4 and/or 16 as compared to placebo group or their baseline.

In one embodiment related to any of the above aspects or their related embodiment(s), at least 10%, 20%, 30%, 40%, 50%, 60%, 70% or 80% of the patients treated with an anti-IL-36R antibody of the present invention achieve 75% improvement in Eczema Area and Severity Index (EASI)(EASI75) at week 4 and/or 16 as compared to placebo group or their baseline.

In one embodiment related to any of the above aspects or their related embodiment(s), at least 10%, 20%, 30%, 40%, 50%, 60%, 70% or 80% of the patients treated with an anti-IL-36R antibody of the present invention experience at least 10%, at least 20%, at least 30%, at least 40%, or at least 50% improvement in SCORing of Atopic Dermatitis (SCORAD) at week 4 and/or 16 as compared to placebo group or their baseline.

In one embodiment related to any of the above aspects or their related embodiment(s), at least 10%, 20%, 30%, 40%, 50%, 60%, 70% or 80% of the patients treated with an anti-IL-36R antibody of the present invention achieve at least a 2-grade reduction to clear (0) or almost clear (1) in Investigator's Global Assessment (IGA) at week 4 and/or 16 as compared to placebo or their baseline.

In an embodiment relating to any of the above aspects, the anti-IL-36R antibody includes: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 53 or 141 (H-CDR1); the amino acid sequence of SEQ ID NO: 62, 108, 109, 110, 111 or 142 (H-CDR2); the amino acid sequence of SEQ ID NO: 72 (H-CDR3).

In an embodiment relating to any of the above aspects, the improved effects (including the remission or improved symptoms) last for 4, 5, 6, 7, 8, 9, 10, 11, 12, 13, 14, 15, 16, 17, 18, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks following the administration of an anti-IL-36R antibody of the present invention.

### Pharmaceutical Compositions and Administration Thereof

The antibodies of the present invention can be administered either alone or in combination with other agents. Examples of antibodies for use in such pharmaceutical compositions are those that comprise an antibody or antibody fragment having the light chain variable region amino acid sequence of any of SEQ ID NO: 1-10. Examples of antibodies for use in such pharmaceutical compositions are also those that comprise a humanized antibody or antibody fragment having the heavy chain variable region amino acid sequence of any of SEQ ID NO: 11-20.

Further examples of antibodies for use in such pharmaceutical compositions are also those that comprise a humanized antibody or antibody fragment having the light chain variable region amino acid sequence of any of SEQ ID NO:76-86. Preferred antibodies for use in such pharmaceutical compositions are also those that comprise a humanized antibody or antibody fragment having the heavy chain variable region amino acid sequence of any of SEQ ID NO:87-101.

Further examples of antibodies for use in such pharmaceutical compositions are also those that comprise a humanized antibody or antibody fragment having the light chain variable region and heavy chain variable region of any of SEQ ID NO: 77 and 89, SEQ ID NO: 80 and 88, SEQ ID NO: 80 and 89, SEQ ID NO: 77 and 87, SEQ ID NO: 77 and 88, SEQ ID NO: 80 and 87, SEQ ID NO: 86 and 100, SEQ ID NO: 85 and 101, or SEQ ID NO: 85 and 10.

Further examples of antibodies for use in such pharmaceutical compositions are also those that comprise a humanized antibody having the light chain region amino acid sequence of any of SEQ ID NO:115, 118, 123 or 124. Preferred antibodies for use in such pharmaceutical compositions are also those that comprise humanized antibody having the heavy chain variable region amino acid sequence of any of SEQ ID NO:125, 126, 127, 138 or 139.

Further examples of antibodies for use in such pharmaceutical compositions are also those that comprise Antibody B1, Antibody B2, Antibody B3, Antibody B4, Antibody B5, Antibody B6, Antibody C1, Antibody C2 or Antibody C3.

Various delivery systems are known and can be used to administer the IL-36R binding agent. Methods of introduction include but are not limited to intradermal, intramuscular, intraperitoneal, intravenous, subcutaneous, intranasal, epidural, and oral routes. The IL-36R binding agent can be administered, for example by infusion, bolus or injection, and can be administered together with other biologically active agents such as chemotherapeutic agents. Administration can be systemic or local. In preferred embodiments, the administration is by subcutaneous injection. Formulations for such injections may be prepared in for example prefilled syringes that may be administered once every other week.

In one aspect, the invention provides an article of manufacture comprising a subcutaneous administration device, which delivers to a patient a fixed dose of an antibody of the present invention. In some embodiments, the subcutaneous administration device is a pre-filled syringe, an autoinjector, or a large volume infusion device. For example, MyDose™ product from Roche, a single use infusion device that enables the subcutaneous administration of large quantities of liquid medication, may be used as the administration device. Numerous reusable pen and autoinjector delivery devices have applications in the subcutaneous delivery of a pharmaceutical composition of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™ OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition of the present invention include, but are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L. P.), and the HUMIRA™ Pen (Abbott Labs, Abbott Park III.), YPSOMATE™, YPSOMATE 2.25™, VAIROJECT™ (Ypsomed AG, Burgdorf, Switzerland) to name only a few. Additional information relating to example delivery devices that could be used with an antibody of the present invention may be found, for example, in CH705992A2, WO2009/040602, WO2016/169748, WO2016/179713.

In specific embodiments, the IL-36R binding agent composition is administered by injection, by means of a catheter, by means of a suppository, or by means of an implant, the implant being of a porous, non-porous, or gelatinous material, including a membrane, such as a sialastic membrane, or a fiber. Typically, when administering the composition, materials to which the anti-IL-36R antibody or agent does not absorb are used.

In other embodiments, the anti-IL-36R antibody or agent is delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.) Other controlled release systems are discussed, for example, in Langer, supra.

An IL-36R binding agent (e.g., an anti-IL-36R antibody) can be administered as pharmaceutical compositions comprising a therapeutically effective amount of the binding agent and one or more pharmaceutically compatible ingredients.

In one embodiment, the anti-IL-36R antibody or an antigen binding fragment thereof (disclosed herein) is present in a pharmaceutical formulation (as described in co-pending PCT application No. PCT/US2020/021059, filed Mar. 5, 2020, the entire content of which is hereby incorporated herein by reference in its entirety) suitable for administration to a subject according to any one of the aspects described herein. Various examples to this embodiment are described as numbered clauses (1, 2, 3, etc.) below for convenience. These are provided as examples and do not limit the subject technology. It is noted that any of the dependent clauses may be combined in any combination, and placed into a respective independent clause, e.g., clause 1. The other clauses can be presented in a similar manner.
1. A method for treating atopic dermatitis (AtD) in a subject; or a method of preventing or ameliorating AtD in a subject; or a method of reducing a microbial colonization of the skin in a subject with AtD; or a method of reducing susceptibility to a skin infection in a subject with AtD; or a method of treating a skin disorder associated with AtD in a subject; or a method of treating skin inflammation associated with AtD in a subject, said method including administering to the subject a dosage regimen of an anti-IL-36R antibody in a pharmaceutical formulation; OR an anti-IL-36R antibody in a pharmaceutical formulation for use in treating atopic dermatitis (AtD) in a subject; or an anti-IL-36R antibody in a pharmaceutical formulation for use in preventing or ameliorating AtD in a subject; or an anti-IL-36R antibody in a pharmaceutical formulation for use in reducing a microbial colonization of the skin in a subject with AtD; or an anti-IL-36R antibody in a pharmaceutical formulation for use in reducing susceptibility to a skin infection in a subject with AtD; or an anti-IL-36R antibody in a pharmaceutical formulation for use in treating a skin disorder associated with AtD in a subject; or an anti-IL-36R antibody in a pharmaceutical formulation for use in treating skin inflammation associated with AtD in a subject; comprising administering to the subject a dosage regimen of; OR
   use of an anti-IL-36R antibody in a pharmaceutical formulation for the manufacture of a medicament for the treatment of atopic dermatitis (AtD) in a subject; or use of an anti-IL-36R antibody in a pharmaceutical formulation for the manufacture of a medicament for preventing or ameliorating AtD in a subject; or use of an anti-IL-36R antibody in a pharmaceutical formulation for the manufacture of a medicament for reducing a microbial colonization of the skin in a subject with AtD; or use of an anti-IL-36R antibody in a pharmaceutical formulation for the manufacture of a medicament for reducing susceptibility to a skin infection in a subject with AtD; or use of an anti-IL-36R antibody in a pharmaceutical formulation for the manufacture of a medicament for treating a skin disorder associated with AtD in a subject; or use of an anti-IL-36R antibody in a pharmaceutical formulation for the manufacture of a medicament for treating skin inflammation associated with AtD in a subject; comprising administering to the subject a dosage regimen of;
   Wherein the anti-IL-36R antibody includes:
      a. a light chain including an amino acid sequence set forth as SEQ ID NO:118 and a heavy chain including an amino acid sequence set forth as SEQ ID NO:125; or
      b. a light chain including an amino acid sequence set forth as SEQ ID NO:118 and a heavy chain including an amino acid sequence set forth as SEQ ID NO:126; or
      c. a light chain including an amino acid sequence set forth as SEQ ID NO:118 and a heavy chain including an amino acid sequence set forth as SEQ ID NO:127;
   wherein the pharmaceutical formulation is selected from the group consisting of:
      I. formulation including about 20 mg/mL of the anti-IL-36R antibody, about 40 mM histidine, about 120 mM sucrose, about 50 mM L-Arginine, about 5 mM NaCl and about 1.0 g/L Polysorbate 20, with a pH of about 6.0;
      II. formulation including about 60 mg/mL of the anti-IL-36R antibody, about 45 mM acetate, about 150 mM sucrose, about 25 mM L-Arginine, about 0.4 g/L Polysorbate 20, with a pH of about 5.5;
      III. formulation including about 20 mg/mL of the anti-IL-36R antibody, about 45 mM acetate, about 180 mM sucrose, about 25 mM Glycine, about 0.4 g/L Polysorbate 80, with a pH of about 5.5;
      IV. formulation including about 150 mg/mL of the anti-IL-36R antibody, about 25 mM citrate, about 150 mM trehalose, about 25 mM methionine, about 0.2 g/L Polysorbate 20, with a pH of about 6.0;
      V. formulation including about 150 mg/mL of the anti-IL-36R antibody, about 25 mM histidine, about 180 mM sucrose, about 20 mM mannitol, about 0.2 g/L Polysorbate 20, with a pH of about 6.5;
      VI. formulation including about 20 mg/mL of the anti-IL-36R antibody, about 25 mM citrate, about 200 mM sucrose, about 0.4 g/L Polysorbate 80, with a pH of about 6.5;
      VII. formulation including about 150 mg/mL of the anti-IL-36R antibody, about 45 mM acetate, about 150 mM sucrose, about 25 mM L-Arginine, about 0.4 g/L Polysorbate 20, with a pH of about 5.5;
      VIII. formulation including about 15 mg/mL of the anti-IL-36R antibody, about 35 mM histidine, about 180 mM trehalose, about 25 mM L-Arginine, about 3 mM NaCl, about 0.4 g/L Polysorbate 80, with a pH of about 6.0;
      IX. formulation including about 80 mg/mL of the anti-IL-36R antibody, about 25 mM acetate, about 100 mM mannitol, about 50 mM NaCl, about 0.2 g/L Polysorbate 20, with a pH of about 5.5;
      X. formulation including about 100 mg/mL of the anti-IL-36R antibody, about 20 mM succinate, about 220 mM sucrose, about 0.1 g/L Polysorbate 80, with a pH of about 6.0; and
      XI. formulation including about 60 mg/mL of the anti-IL-36R antibody, about 25 mM citrate, about 0.4 g/L Polysorbate 20, with a pH of about 6.5;
   wherein the dosage regimen includes:
      a. subcutaneous administrations of one or more doses of 300 mg or one or more doses of 600 mg each of the anti-IL-36R antibody once every week(qw), once every 2 weeks (q2w), once every 4 weeks (q4w), once every 6 weeks (q6w) or once every 8 weeks (q8w); or
      b. subcutaneous administrations of the anti-IL-36R antibody in an initial dose and a subsequent dose;
         (i) wherein the initial dose incudes:
         i. one or more doses of 150 mg each of the anti-IL-36R antibody administered daily for 2 weeks; or
         ii. one or more doses of 300 mg each of the anti-IL-36R antibody administered daily for 2 weeks; or
         iii. one or more doses of 600 mg each of the anti-IL-36R antibody administered twice, three times or four times in 4 weeks or administered twice per week for 2 weeks, or administered twice per week for 3 weeks, or administered twice per week for 4 weeks; or
         iv. one or two doses of 900 mg or 1200 mg each of the anti-IL-36R antibody administered once only or two times in three weeks; and
         (ii) wherein the subsequent dose includes:
         i. one or more doses of 300 mg each of the anti-IL-36R antibody administered q2w, q4w, q6w or q8w; or
         ii. one or more doses of 600 mg each of the anti-IL-36R antibody administered q2w, q4w, q6w or q8w; and
         (iii) wherein the administration of the subsequent dose is between 2 to 4 weeks or 2 weeks or 4 weeks after the administration of the last initial dose.
2. A method for treating atopic dermatitis (AtD) in a subject; or a method of preventing or ameliorating AtD in a subject; or a method of reducing a microbial colonization of the skin in a subject with AtD; or a method of reducing susceptibility to a skin infection in a subject with AtD; or a method of treating a skin disorder associated with AtD in a subject; or a method of treating skin inflammation associated with AtD in a subject, said method including administering to the subject a dosage regimen of an anti-IL-36R antibody in a pharmaceutical formulation; OR
   an anti-IL-36R antibody in a pharmaceutical formulation for use in treating atopic dermatitis (AtD) in a subject; or an anti-IL-36R antibody in a pharmaceutical formulation for use in preventing or ameliorating AtD in a subject; or an anti-IL-36R antibody in a pharmaceutical formulation for use in reducing a microbial colonization of the skin in a subject with AtD; or an anti-IL-36R antibody in a pharmaceutical formulation for use in reducing susceptibility to a skin infection in a subject with AtD; or an anti-IL-36R antibody in a pharmaceutical formulation for use in treating a skin disorder associated with AtD in a subject; or an anti-IL-36R antibody in a pharmaceutical formulation for use in treating skin inflammation associated with AtD in a subject; comprising administering to the subject a dosage regimen of; OR
   use of an anti-IL-36R antibody in a pharmaceutical formulation for the manufacture of a medicament for the treatment of atopic dermatitis (AtD) in a subject; or use of an anti-IL-36R antibody in a pharmaceutical formulation for the manufacture of a medicament for preventing or ameliorating AtD in a subject; or use of an anti-IL-36R antibody in a pharmaceutical formulation for the manufacture of a medicament for reducing a microbial colonization of the skin in a subject with AtD; or use of an anti-IL-36R antibody in a pharmaceutical formulation for the manufacture of a medicament for reducing susceptibility to a skin infection in a subject with AtD; or use of an anti-IL-36R antibody in a pharmaceutical formulation for the manufacture of a medicament for treating a skin disorder associated with AtD in a subject; or use of an anti-IL-36R antibody in a pharmaceutical formulation for the manufacture of a medicament for treating skin inflammation associated with AtD in a subject; comprising administering to the subject a dosage regimen of;
      Wherein the anti-IL-36R antibody includes:
   (i) a light chain variable region comprising the amino acid sequence of SEQ ID NO: 77; and a heavy chain variable region comprising the amino acid sequence of SEQ ID NO: 87; or
   (ii) a light chain variable region comprising the amino acid sequence of SEQ ID NO: 77; and a heavy chain variable region comprising the amino acid sequence of SEQ ID NO: 88; or
   (iii) a light chain variable region comprising the amino acid sequence of SEQ ID NO: 77; and a heavy chain variable region comprising the amino acid sequence of SEQ ID NO: 89; or
   (iv) a light chain variable region comprising the amino acid sequence of SEQ ID NO: 80; and a heavy chain variable region comprising the amino acid sequence of SEQ ID NO: 87; or
   (v) a light chain variable region comprising the amino acid sequence of SEQ ID NO: 80; and a heavy chain variable region comprising the amino acid sequence of SEQ ID NO: 88; or
   (vi) a light chain variable region comprising the amino acid sequence of SEQ ID NO: 80; and a heavy chain variable region comprising the amino acid sequence of SEQ ID NO: 89;
      wherein the formulation is selected from the group consisting of:
         I. formulation including about 20 mg/mL of the anti-IL-36R antibody, about 40 mM histidine, about 120 mM sucrose, about 50 mM L-Arginine, about 5 mM NaCl and about 1.0 g/L Polysorbate 20, with a pH of about 6.0;
         II. formulation including about 60 mg/mL of the anti-IL-36R antibody, about 45 mM acetate, about 150 mM sucrose, about 25 mM L-Arginine, about 0.4 g/L Polysorbate 20, with a pH of about 5.5;
         III. formulation including about 20 mg/mL of the anti-IL-36R antibody, about 45 mM acetate, about 180 mM sucrose, about 25 mM Glycine, about 0.4 g/L Polysorbate 80, with a pH of about 5.5;
         IV. formulation including about 150 mg/mL of the anti-IL-36R antibody, about 25 mM citrate, about 150 mM trehalose, about 25 mM methionine, about 0.2 g/L Polysorbate 20, with a pH of about 6.0;
         V. formulation including about 150 mg/mL of the anti-IL-36R antibody, about 25 mM histidine, about 180 mM sucrose, about 20 mM mannitol, about 0.2 g/L Polysorbate 20, with a pH of about 6.5;
         VI. formulation including about 20 mg/mL of the anti-IL-36R antibody, about 25 mM citrate, about 200 mM sucrose, about 0.4 g/L Polysorbate 80, with a pH of about 6.5;
         VII. formulation including about 150 mg/mL of the anti-IL-36R antibody, about 45 mM acetate, about 150 mM sucrose, about 25 mM L-Arginine, about 0.4 g/L Polysorbate 20, with a pH of about 5.5;
         VIII. formulation including about 15 mg/mL of the anti-IL-36R antibody, about 35 mM histidine, about 180 mM trehalose, about 25 mM L-Arginine, about 3 mM NaCl, about 0.4 g/L Polysorbate 80, with a pH of about 6.0;
         IX. formulation including about 80 mg/mL of the anti-IL-36R antibody, about 25 mM acetate, about 100 mM mannitol, about 50 mM NaCl, about 0.2 g/L Polysorbate 20, with a pH of about 5.5;
         X. formulation including about 100 mg/mL of the anti-IL-36R antibody, about 20 mM succinate, about 220 mM sucrose, about 0.1 g/L Polysorbate 80, with a pH of about 6.0; and
         X. formulation including about 60 mg/mL of the anti-IL-36R antibody, about 25 mM citrate, about 0.4 g/L Polysorbate 20, with a pH of about 6.5;
      wherein the dosage regimen includes:
         a. subcutaneous administrations of one or more doses of 300 mg or one or more doses of 600 mg each of the anti-IL-36R antibody once every week(qw), once every 2 weeks (q2w), once every 4 weeks (q4w), once every 6 weeks (q6w) or once every 8 weeks (q8w); or
         b. subcutaneous administrations of the anti-IL-36R antibody in an initial dose and a subsequent dose;
         (i) wherein the initial dose incudes:
         i. one or more doses of 150 mg each of the anti-IL-36R antibody administered daily for 2 weeks; or
         ii. one or more doses of 300 mg each of the anti-IL-36R antibody administered daily for 2 weeks; or
         iii. one or more doses of 600 mg each of the anti-IL-36R antibody administered twice, three times or four times in 4 weeks or administered twice per week for 2 weeks, or administered twice per week for 3 weeks, or administered twice per week for 4 weeks; or
         iv. one or two doses of 900 mg or 1200 mg each of the anti-IL-36R antibody administered once only or two times in three weeks; and
         (ii) wherein the subsequent dose includes:
         i. one or more doses of 300 mg each of the anti-IL-36R antibody administered q2w, q4w, q6w or q8w; or
         ii. one or more doses of 600 mg each of the anti-IL-36R antibody administered q2w, q4w, q6w or q8w; and
         (iii) wherein the administration of the subsequent dose is between 2 to 4 weeks or 2 weeks or 4 weeks after the administration of the last initial dose.
3. A method for treating atopic dermatitis (AtD) in a subject; or a method of preventing or ameliorating AtD in a subject; or a method of reducing a microbial colonization of the skin in a subject with AtD; or a method of reducing susceptibility to a skin infection in a subject with AtD; or a method of treating a skin disorder associated with AtD in a subject; or a method of treating skin inflammation associated with AtD in a subject, said method including administering to the subject a dosage regimen of an anti-IL-36R antibody in a pharmaceutical formulation; OR
   an anti-IL-36R antibody in a pharmaceutical formulation for use in treating atopic dermatitis (AtD) in a subject; or an anti-IL-36R antibody in a pharmaceutical formulation for use in preventing or ameliorating AtD in a subject; or an anti-IL-36R antibody in a pharmaceutical formulation for use in reducing a microbial colonization of the skin in a subject with AtD; or an anti-IL-36R antibody in a pharmaceutical formulation for use in reducing susceptibility to a skin infection in a subject with AtD; or an anti-IL-36R antibody in a pharmaceutical formulation for use in treating a skin disorder associated with AtD in a subject; or an anti-IL-36R antibody in a pharmaceutical formulation for use in treating skin inflammation associated with AtD in a subject; comprising administering to the subject a dosage regimen of; OR
   use of an anti-IL-36R antibody in a pharmaceutical formulation for the manufacture of a medicament for the treatment of atopic dermatitis (AtD) in a subject; or use of an anti-IL-36R antibody in a pharmaceutical formulation for the manufacture of a medicament for preventing or ameliorating AtD in a subject; or use of an anti-IL-36R antibody in a pharmaceutical formulation for the manufacture of a medicament for reducing a microbial colonization of the skin in a subject with AtD; or use of an anti-IL-36R antibody in a pharmaceutical formulation for the manufacture of a medicament for reducing susceptibility to a skin infection in a subject with AtD; or use of an anti-IL-36R antibody in a pharmaceutical formulation for the manufacture of a medicament for treating a skin disorder associated with AtD in a subject; or use of an anti-IL-36R antibody in a pharmaceutical formulation for the manufacture of a medicament for treating skin inflammation associated with AtD in a subject; comprising administering to the subject a dosage regimen of;
      Wherein the anti-IL-36R antibody includes:
         a. a light chain including an amino acid sequence set forth as SEQ ID NO:118 and a heavy chain including an amino acid sequence set forth as SEQ ID NO:127;
      wherein the pharmaceutical formulation is selected from the group consisting of:
         I. formulation including about 20 mg/mL of the anti-IL-36R antibody, about 40 mM histidine, about 120 mM sucrose, about 50 mM L-Arginine, about 5 mM NaCl and about 1.0 g/L Polysorbate 20, with a pH of about 6.0;
         II. formulation including about 60 mg/mL of the anti-IL-36R antibody, about 45 mM acetate, about 150 mM sucrose, about 25 mM L-Arginine, about 0.4 g/L Polysorbate 20, with a pH of about 5.5;
         III. formulation including about 20 mg/mL of the anti-IL-36R antibody, about 45 mM acetate, about 180 mM sucrose, about 25 mM Glycine, about 0.4 g/L Polysorbate 80, with a pH of about 5.5;
         IV. formulation including about 150 mg/mL of the anti-IL-36R antibody, about 25 mM citrate, about 150 mM trehalose, about 25 mM methionine, about 0.2 g/L Polysorbate 20, with a pH of about 6.0;
         V. formulation including about 150 mg/mL of the anti-IL-36R antibody, about 25 mM histidine, about 180 mM sucrose, about 20 mM mannitol, about 0.2 g/L Polysorbate 20, with a pH of about 6.5;
         VI. formulation including about 20 mg/mL of the anti-IL-36R antibody, about 25 mM citrate, about 200 mM sucrose, about 0.4 g/L Polysorbate 80, with a pH of about 6.5;
         VII. formulation including about 150 mg/mL of the anti-IL-36R antibody, about 45 mM acetate, about 150 mM sucrose, about 25 mM L-Arginine, about 0.4 g/L Polysorbate 20, with a pH of about 5.5;
         VIII. formulation including about 15 mg/mL of the anti-IL-36R antibody, about 35 mM histidine, about 180 mM trehalose, about 25 mM L-Arginine, about 3 mM NaCl, about 0.4 g/L Polysorbate 80, with a pH of about 6.0;
         IX. formulation including about 80 mg/mL of the anti-IL-36R antibody, about 25 mM acetate, about 100 mM mannitol, about 50 mM NaCl, about 0.2 g/L Polysorbate 20, with a pH of about 5.5;
         X. formulation including about 100 mg/mL of the anti-IL-36R antibody, about 20 mM succinate, about 220 mM sucrose, about 0.1 g/L Polysorbate 80, with a pH of about 6.0; and
         XI. formulation including about 60 mg/mL of the anti-IL-36R antibody, about 25 mM citrate, about 0.4 g/L Polysorbate 20, with a pH of about 6.5;
      wherein the dosage regimen includes:
         a. subcutaneous administrations of one or more doses of 300 mg or one or more doses of 600 mg each of the anti-IL-36R antibody once every week(qw), once every 2 weeks (q2w), once every 4 weeks (q4w), once every 6 weeks (q6w) or once every 8 weeks (q8w); or
         b. subcutaneous administrations of the anti-IL-36R antibody in an initial dose and a subsequent dose;
         (iv) wherein the initial dose incudes:
         i. one or more doses of 150 mg each of the anti-IL-36R antibody administered daily for 2 weeks; or
         ii. one or more doses of 300 mg each of the anti-IL-36R antibody administered daily for 2 weeks; or
         iii. one or more doses of 600 mg each of the anti-IL-36R antibody administered twice, three times or four times in 4 weeks or administered twice per week for 2 weeks, or administered twice per week for 3 weeks, or administered twice per week for 4 weeks; or
         iv. one or two doses of 900 mg or 1200 mg each of the anti-IL-36R antibody administered once only or two times in three weeks; and
         (v) wherein the subsequent dose includes:
         i. one or more doses of 300 mg each of the anti-IL-36R antibody administered q2w, q4w, q6w or q8w; or
         ii. one or more doses of 600 mg each of the anti-IL-36R antibody administered q2w, q4w, q6w or q8w; and
         (vi) wherein the administration of the subsequent dose is between 2 to 4 weeks, 2 weeks, or 4 weeks after the administration of the last initial dose.
4. A method for treating atopic dermatitis (AtD) in a subject; or a method of preventing or ameliorating AtD in a subject; or a method of reducing a microbial colonization of the skin in a subject with AtD; or a method of reducing susceptibility to a skin infection in a subject with AtD; or a method of treating a skin disorder associated with AtD in a subject; or a method of treating skin inflammation associated with AtD in a subject, said method including administering to the subject a dosage regimen of an anti-IL-36R antibody in a pharmaceutical formulation; OR
   an anti-IL-36R antibody in a pharmaceutical formulation for use in treating atopic dermatitis (AtD) in a subject; or an anti-IL-36R antibody in a pharmaceutical formulation for use in preventing or ameliorating AtD in a subject; or an anti-IL-36R antibody in a pharmaceutical formulation for use in reducing a microbial colonization of the skin in a subject with AtD; or an anti-IL-36R antibody in a pharmaceutical formulation for use in reducing susceptibility to a skin infection in a subject with AtD; or an anti-IL-36R antibody in a pharmaceutical formulation for use in treating a skin disorder associated with AtD in a subject; or an anti-IL-36R antibody in a pharmaceutical formulation for use in treating skin inflammation associated with AtD in a subject; comprising administering to the subject a dosage regimen of; OR
   use of an anti-IL-36R antibody in a pharmaceutical formulation for the manufacture of a medicament for the treatment of atopic dermatitis (AtD) in a subject; or use of an anti-IL-36R antibody in a pharmaceutical formulation for the manufacture of a medicament for preventing or ameliorating AtD in a subject; or use of an anti-IL-36R antibody in a pharmaceutical formulation for the manufacture of a medicament for reducing a microbial colonization of the skin in a subject with AtD; or use of an anti-IL-36R antibody in a pharmaceutical formulation for the manufacture of a medicament for reducing susceptibility to a skin infection in a subject with AtD; or use of an anti-IL-36R antibody in a pharmaceutical formulation for the manufacture of a medicament for treating a skin disorder associated with AtD in a subject; or use of an anti-IL-36R antibody in a pharmaceutical formulation for the manufacture of a medicament for treating skin inflammation associated with AtD in a subject; comprising administering to the subject a dosage regimen of;
      Wherein the anti-IL-36R antibody includes:
         (i) a light chain variable region comprising the amino acid sequence of SEQ ID NO: 80; and a heavy chain variable region comprising the amino acid sequence of SEQ ID NO: 89;
      wherein the formulation is selected from the group consisting of:
         I. formulation including about 20 mg/mL of the anti-IL-36R antibody, about 40 mM histidine, about 120 mM sucrose, about 50 mM L-Arginine, about 5 mM NaCl and about 1.0 g/L Polysorbate 20, with a pH of about 6.0;
         II. formulation including about 60 mg/mL of the anti-IL-36R antibody, about 45 mM acetate, about 150 mM sucrose, about 25 mM L-Arginine, about 0.4 g/L Polysorbate 20, with a pH of about 5.5;
         III. formulation including about 20 mg/mL of the anti-IL-36R antibody, about 45 mM acetate, about 180 mM sucrose, about 25 mM Glycine, about 0.4 g/L Polysorbate 80, with a pH of about 5.5;
         IV. formulation including about 150 mg/mL of the anti-IL-36R antibody, about 25 mM citrate, about 150 mM trehalose, about 25 mM methionine, about 0.2 g/L Polysorbate 20, with a pH of about 6.0;
         V. formulation including about 150 mg/mL of the anti-IL-36R antibody, about 25 mM histidine, about 180 mM sucrose, about 20 mM mannitol, about 0.2 g/L Polysorbate 20, with a pH of about 6.5;
         VI. formulation including about 20 mg/mL of the anti-IL-36R antibody, about 25 mM citrate, about 200 mM sucrose, about 0.4 g/L Polysorbate 80, with a pH of about 6.5;
         VII. formulation including about 150 mg/mL of the anti-IL-36R antibody, about 45 mM acetate, about 150 mM sucrose, about 25 mM L-Arginine, about 0.4 g/L Polysorbate 20, with a pH of about 5.5;
         VIII. formulation including about 15 mg/mL of the anti-IL-36R antibody, about 35 mM histidine, about 180 mM trehalose, about 25 mM L-Arginine, about 3 mM NaCl, about 0.4 g/L Polysorbate 80, with a pH of about 6.0;
         IX. formulation including about 80 mg/mL of the anti-IL-36R antibody, about 25 mM acetate, about 100 mM mannitol, about 50 mM NaCl, about 0.2 g/L Polysorbate 20, with a pH of about 5.5;
         X. formulation including about 100 mg/mL of the anti-IL-36R antibody, about 20 mM succinate, about 220 mM sucrose, about 0.1 g/L Polysorbate 80, with a pH of about 6.0; and
         XI. formulation including about 60 mg/mL of the anti-IL-36R antibody, about 25 mM citrate, about 0.4 g/L Polysorbate 20, with a pH of about 6.5;
      wherein the dosage regimen includes:
         a. subcutaneous administrations of one or more doses of 300 mg or one or more doses of 600 mg each of the anti-IL-36R antibody once every week(qw), once every 2 weeks (q2w), once every 4 weeks (q4w), once every 6 weeks (q6w) or once every 8 weeks (q8w); or
         b. subcutaneous administrations of the anti-IL-36R antibody in an initial dose and a subsequent dose;
         (i) wherein the initial dose incudes:
         i. one or more doses of 150 mg each of the anti-IL-36R antibody administered daily for 2 weeks; or
         ii. one or more doses of 300 mg each of the anti-IL-36R antibody administered daily for 2 weeks; or
         iii. one or more doses of 600 mg each of the anti-IL-36R antibody administered twice, three times or four times in 4 weeks or administered twice per week for 2 weeks, or administered twice per week for 3 weeks, or administered twice per week for 4 weeks; or
         iv. one or two doses of 900 mg or 1200 mg each of the anti-IL-36R antibody administered once only or two times in three weeks; and
         (ii) wherein the subsequent dose includes:
         i. one or more doses of 300 mg each of the anti-IL-36R antibody administered q2w, q4w, q6w or q8w; or
         ii. one or more doses of 600 mg each of the anti-IL-36R antibody administered q2w, q4w, q6w or q8w; and
         (iii) wherein the administration of the subsequent dose is between 2 to 4 weeks or 2 weeks or 4 weeks after the administration of the last initial dose.
5. The method, the anti-IL36R antibody for use, or the use for manufacture according to any of clauses 1-5, wherein the colonization is of a microbe selected from the group consisting of Staphylococcus aureus, Streptococcus spp., Pseudomonas aeruginosa, Bacteroides spp., molluscum contagiosum virus, Herpes simplex virus, coxsackievirus, vaccinia virus, Candida albicans, Microsporum spp., Trichophyton spp., Penicillium spp., Cladosporium spp., Alternaria spp., and Aspergillus spp.
6. The method, the anti-IL36R antibody for use, or the use for manufacture of clause 5, wherein the microbe is Staphylococcus aureus (S. aureus).
7. The method, the anti-IL36R antibody for use, or the use for manufacture of clause 6, wherein the S. aureus colonization is reduced by at least 20% from the baseline.
8. The method, the anti-IL36R antibody for use, or the use for manufacture according to any of clauses 1-5, wherein the skin infection is caused by a microbe selected from the group consisting of Staphylococcus aureus, Streptococcus spp., Pseudomonas aeruginosa, Bacteroides spp., Herpes simplex virus, molluscum contagiosum virus, coxsackievirus, vaccinia virus, Candida albicans, Microsporum spp., Trichophyton spp., Penicillium spp., Cladosporium spp., Alternaria spp., and Aspergillus spp.
9. The method, the anti-IL36R antibody for use, or the use for manufacture of clause 8, wherein the microbe is Staphylococcus aureus (S. aureus).
10. The method, the anti-IL36R antibody for use, or the use for manufacture according to any of clauses 1-5, wherein a second therapeutic agent is administered to the subject before, after, or concurrent with the anti-IL-36R antibody.
11. The method, the anti-IL36R antibody for use, or the use for manufacture of clause 10, wherein the second therapeutic agent is selected from the group consisting of an anti-bacterial agent, an anti-viral agent, an anti-fungal agent, an anti-IL-36R antibody, an IgE inhibitor, a corticosteroid, a non-steroid anti-inflammatory drug (NSAID), an IL-4R antagonist, and IFN-γ.
12. The method, the anti-IL36R antibody for use, or the use for manufacture according to clauses 1 to 5, wherein the pharmaceutical formulation is in a vial, syringe with or without a needle safety device, or a an autoinjector.
13. The method, the anti-IL36R antibody for use, or the use for manufacture according to clause 12, wherein the autoinjector or the syringe with a needle safety device includes:
   a. about 300 mg of the antibody in about 2 mL formulation volume; or
   b. about 225 mg of the antibody in about 1.5 mL formulation volume; or
   c. about 150 mg of the antibody in about 1 mL formulation volume; or
   d. about 75 mg of the antibody in about 0.5 mL formulation volume; or
   e. about 60 mg of the antibody in about 0.4 mL formulation volume.
14. The method, the anti-IL36R antibody for use, or the use for manufacture according to clause 12, wherein the vial includes:
   a. about 1200 mg of the antibody in about 20 mL formulation volume; or
   b. about 900 mg of the antibody in about 15 mL formulation volume; or
   c. about 600 mg of the antibody in about 10 mL formulation volume; or
   d. about 300 mg of the antibody in about 150 mL formulation volume; or
   e. about 1500 mg of the antibody in about 2.5 mL formulation volume.
15. The method, the anti-IL36R antibody for use, or the use for manufacture according to any of clauses 1-5, wherein the treatment results in an improvement in the subject; wherein the improvement is determined by an endpoint selected from the group consisting of: (i) positive changes in Eczema Area and Severity Index (EASI) score at 16 weeks after the treatment; (ii) attaining an EASI 50 at 16 weeks after the treatment; (iii) attaining an EASI 75 at week16 after the treatment; and (iv) a positive change in Scoring Atopic Dermatitis (SCORAD), Max Itch Intensity or Dermatology Life Quality Index (DLQI).
16. The method, the anti-IL36R antibody for use, or the use for manufacture according to any of the preceding clauses, wherein the treatment results in one or more of the following outcomes in the subject as compared to the subject's conditions at baseline or before the treatment or as compared to placebo:
   i. at least 10% improvement in Eczema Area and Severity Index (EASI) score at 4 and/or 16 weeks;
   ii. at least 10% improvement in proportion of patients who attain EASI 50 at 4 and/or 16 weeks;
   iii. at least 10% improvement in proportion of patients who attain EASI 75 at 4 and/or 16 weeks;
   iv. at least 10% improvement in SCORAD, Max Itch Intensity and DLQI;
   v. at least 10% improvement in number of patients with drug related Adverse Events (AEs) up to week 44;
   vi. at least 10% improvement in absolute and percentage change from baseline in Eczema Area and Severity Index (EASI) at week 4;
   vii. at least 10% improvement in proportion of patients with a 50% improvement in Eczema Area and Severity Index (EASI)(EASI50) at weeks 4 and/or 16;
   viii. at least 5% improvement in proportion of patients with a 75% improvement in Eczema Area and Severity Index (EASI)(EASI75) at week 4 and/or 16;
   ix. at least 10% improvement in SCORing of Atopic Dermatitis (SCORAD) at week 4 and/or 16;
   x. at least 5% improvement in proportion of patients achieving at least a 2-grade reduction to clear (0) or almost clear (1) in Investigator's Global Assessment (IGA) at week 4 and/or 16;
   xi. at least 10 percentage point difference in percentage change from baseline in EASI score at week 16;
   xii. at least 10 percentage point difference in EASI50 response rate at week 16;
   xiii. at least 5 percentage point difference in EASI75 response rate at week 16;
   xiv. at least 10 percentage point difference in percentage change from baseline in SCORAD, Max Itch Intensity and DLQI at week 16;
   xv. at least 10 percentage point difference in percentage change from baseline in Eczema Area and Severity Index (EASI) at week 44; or
   xvi. at least 5 percentage point difference in IGA rate at week 16.

Further, the pharmaceutical composition can be provided as a pharmaceutical kit comprising (a) a container containing a IL-36R binding agent (e.g., an anti-IL-36R antibody) in lyophilized form and (b) a second container containing a pharmaceutically acceptable diluent (e.g., sterile water) for injection. The pharmaceutically acceptable diluent can be used for reconstitution or dilution of the lyophilized anti-IL-36R antibody or agent. Optionally associated with such container(s) can be a notice in the form prescribed by a governmental agency regulating the manufacture, use or sale of pharmaceuticals or biological products, which notice reflects approval by the agency of manufacture, use or sale for human administration.

### Combination Therapies

The methods of the present invention, according to certain embodiments, comprise administering to the subject one or more additional therapeutic agents in combination with the anti-IL-36R antibody. As used herein, the expression “in combination with” means that the additional therapeutic agents are administered before, after, or concurrent with the pharmaceutical composition comprising the anti-IL-36R antibody. The term “in combination with” also includes sequential or concomitant administration of an anti-IL-36R antibody and a second therapeutic agent.

For example, when administered “before” the pharmaceutical composition comprising the anti-IL-36R antibody, the additional therapeutic agent may be administered about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes or about 10 minutes prior to the administration of the pharmaceutical composition comprising the anti-IL-36R antibody. When administered “after” the pharmaceutical composition comprising the anti-IL-36R antibody, the additional therapeutic agent may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours or about 72 hours after the administration of the pharmaceutical composition comprising the anti-IL-36R antibody. Administration “concurrent” or with the pharmaceutical composition comprising the anti-IL-36R antibody means that the additional therapeutic agent is administered to the subject in a separate dosage form within less than 5 minutes (before, after, or at the same time) of administration of the pharmaceutical composition comprising the anti-IL-36R antibody, or administered to the subject as a single combined dosage formulation comprising both the additional therapeutic agent and the anti-IL-36R antibody.

The additional therapeutic agent may be, e.g., an anti-bacterial agent (including topical and systemic antibiotics, broad-spectrum and narrow-spectrum antibiotics), an anti-viral agent (e.g., acyclovir, or foscarnet), an anti-fungal agent (e.g., fluconazole and econazole nitrate), an IL-4R antagonist, an IgE antagonist, interferon-gamma (IFNγ) antibiotics, topical antiseptic lotion, or any other emollient therapy or combinations thereof.

The methods of the invention comprise administering an anti-IL-36R antibody or an antigen binding fragment thereof (disclosed herein) in combination with a second therapeutic agent for additive or synergistic activity to reduce the risk of skin infections, e.g., in a patient with AtD.

According to certain embodiments of the present invention, a single or multiple doses of an anti-IL-36R antibody may be administered to a subject over a defined time course. The methods according to this aspect of the invention comprise sequentially administering to a subject multiple doses of an anti-IL-36R antibody. As used herein, “sequentially administering” means that each dose of the anti-IL-36R antibody is administered to the subject at a different point in time, e.g., on different days separated by a predetermined interval (e.g., hours, days, weeks or months). The present invention includes methods which comprise sequentially administering to the patient a single initial dose of an anti-IL-36R antibody, followed by one or more subsequent doses of the anti-IL-36R antibody.

In one exemplary embodiment of the present invention, each subsequent dose is administered at a predetermined interval comprising hours, days, weeks or months after the immediately preceding dose. The phrase “the immediately preceding dose,” as used herein, means, in a sequence of multiple administrations, the dose of anti-IL-36R antibody which is administered to a patient prior to the administration of the very next dose in the sequence with no intervening doses.

The methods according to this aspect of the invention may comprise administering to a patient any number of subsequent doses of an anti-IL-36R antibody. For example, in certain embodiments, only a single secondary dose is administered to the patient. In other embodiments, one or more (e.g., 2, 3, 4, 5, 6, 7, 8, or more) subsequent doses are administered to the patient.

Administration or Dosage Regimens

According to certain embodiments of the present invention, a single or multiple doses of an anti-IL-36R antibody may be administered to a subject over a defined time course. The methods according to this aspect of the invention comprise sequentially administering to a subject multiple doses of an anti-IL-36R antibody. As used herein, “sequentially administering” means that each dose of the anti-IL-36R antibody is administered to the subject at a different point in time, e.g., on different days separated by a predetermined interval (e.g., hours, days, weeks or months). The present invention includes methods which comprise sequentially administering to the patient a single initial dose of an anti-IL-36R antibody, followed by one or more subsequent doses of the anti-IL-36R antibody.

In one exemplary embodiment of the present invention, each subsequent dose is administered at a predetermined interval comprising hours, days, weeks or months after the immediately preceding dose. The phrase “the immediately preceding dose,” as used herein, means, in a sequence of multiple administrations, the dose of anti-IL-36R antibody which is administered to a patient prior to the administration of the very next dose in the sequence with no intervening doses.

The methods according to this aspect of the invention may comprise administering to a patient any number of subsequent doses of an anti-IL-36R antibody. For example, in certain embodiments, only a single secondary dose is administered to the patient. In other embodiments, one or more (e.g., 2, 3, 4, 5, 6, 7, 8, or more) subsequent doses are administered to the patient.

In an embodiment relating to any aspect or embodiment of the present invention, the dosage regimen is any of the regimens listed in Tables 1 and 2. In another embodiment, the dosage regimen the dosage regimen includes: (a) subcutaneous administrations of one or more doses of 300 mg or one or more doses of 600 mg each of the anti-IL-36R antibody once every week(qw), once every 2 weeks (q2w), once every 4 weeks (q4w), once every 6 weeks (q6w) or once every 8 weeks (q8w); or (b) subcutaneous administrations of the anti-IL-36R antibody in an initial dose and a subsequent dose; (i) wherein the initial dose incudes: (1) one or more doses of 150 mg each of the anti-IL-36R antibody administered daily for 2 weeks; or (2) one or more doses of 300 mg each of the anti-IL-36R antibody administered daily for 2 weeks; or (3) one or more doses of 600 mg each of the anti-IL-36R antibody administered twice, three times or four times in 4 weeks or administered twice per week for 2 weeks, or administered twice per week for 3 weeks, or administered twice per week for 4 weeks; or (4) one dose of 900 mg or 1200 mg of the anti-IL-36R antibody administered once; or (5) two doses of 900 mg or 1200 mg each of the anti-IL-36R antibody administered twice in three weeks (e.g., in weeks 0 and 2); (ii) wherein the subsequent dose includes: (1) one or more doses of 300 mg each of the anti-IL-36R antibody administered q2w, q4w, q6w or q8w; or (2) one or more doses of 600 mg each of the anti-IL-36R antibody administered q2w, q4w, q6w or q8w; and (iii) wherein the administration of the subsequent dose is between 2 to 4 weeks after the administration of the last initial dose.

### Articles of Manufacture

In another aspect, an article of manufacture containing materials useful for the treatment of the disorders described above is included. The article of manufacture comprises a container and a label. Suitable containers include, for example, bottles, vials, syringes, and test tubes. The containers may be formed from a variety of materials such as glass or plastic. The container holds a composition that is effective for treating the condition and may have a sterile access port. For example, the container may be an intravenous solution bag or a vial having a stopper pierceable by a hypodermic injection needle. The active agent in the composition is the humanized anti-IL-36R antibody. The label on or associated with the container indicates that the composition is used for treating the condition of choice. The article of manufacture may further comprise a second container comprising a pharmaceutically-acceptable buffer, such as phosphate-buffered saline, Ringer's solution, and dextrose solution. It may further include other materials desirable from a commercial and user standpoint, including other buffers, diluents, filters, needles, syringes, and package inserts with instructions for use.

The invention is further described in the following examples, which are not intended to limit the scope of the invention.

### EXAMPLES

### Example 1: Therapeutic Activity of a Mouse Anti-IL36R Blocking Antibody in Inhibiting Atopic Dermatitis-Like Skin Inflammation in Mice

Staphylococcus aureus epicutaneous exposure to mouse skin was previously found to induce atopic dermatitis (AtD)-like inflammation that was dependent upon IL-36 receptor (IL-36R) activity and inhibitable by subcutaneous administration of IL-36R ab. However, whether systemic treatment with an anti-IL-36R blocking monoclonal antibody (mAb) could have a therapeutic effect by diminishing IL-36-dependent S. aureus-induced skin inflammation is unclear.

In this example, we evaluated the efficacy of an IL-36R blocking mAb of the present invention in inhibiting AtD-like inflammation induced by S. aureus epicutaneous exposure to mouse skin. S. aureus epicutaneous exposure was performed by applying a S. aureus (1×10″8 CFU) soaked gauze pad for 7 days on dorsal skin while the mice were treated with systemic administration (intraperitoneal) of an anti-IL-36R blocking mAb or isotype control mAb on days −1, 2 and 5.

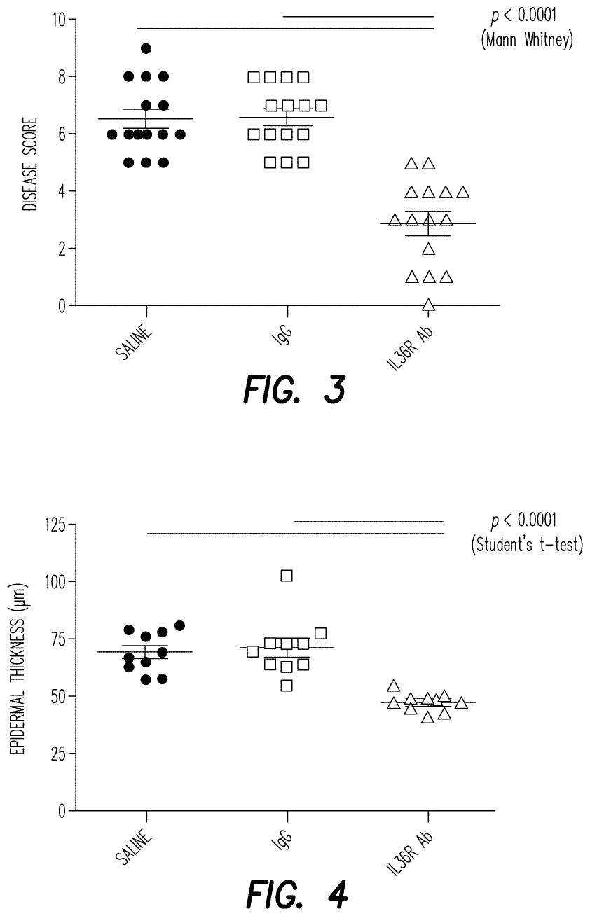

As shown in FIGS. 3 and 4, the anti-IL-36R blocking mAb treatment resulted in significantly decreased AD-like skin inflammation as measured by disease score (edema, erythema, necrosis and scaling) by a blinded observed and epidermal thickness quantified from histologic sections. These data demonstrate the ability of systemically delivered anti-IL-36R blocking antibody to reduce skin inflammation in a mouse model of AD-like inflammation induced by epicutaneous exposure to Staphylococcus aureus.

### Example 2: Inhibition of IL-8 Production from IL-36 γ Stimulated Reconstructed Human Epidermis

### Protocol Reconstructed Epidermis

Anti-IL-36R antibodies (1.5 μg/ml) were pre-incubated with reconstructed human epidermis and stimulated with human recombinant IL-36γ (20 ng/ml). Recombinant human IL-1p (20 ng/ml; R & D Systems) was used as a positive control. After 24 hours in culture, cell supernatants were collected and assayed for IL-8 (assays for IL-8 are described in Example 3). Samples were tested in triplicate and the average pg/ml±standard error is shown in the table below (Table 3).

TABLE 3

| | Cytokine | Average IL-8 (pg/ml) +/− |
|---|---|---|
| Antibody | Stimulation | Standard Error |
| No antibody | None | 57.3 ± 15.3 |
| 33D10 | None | 15.8 ± 0.7 |
| No antibody | 20 ng/mL IL-1 β | 158.9 ± 13.3 |
| 33D10 | 20 ng/mL IL-1 β | 168.5 ± 22.6 |
| No antibody | 20 ng/mL IL-36γ | 142.1 ± 22.2 |
| 33D10 | 20 ng/mL IL-36γ | 38.63 ± 6.7 |

### Example 3: Inhibition of IL-36 Ligand Induced S100A7 and S100A12 Gene Expression in Reconstructed Human Epidermis

Stimulation of reconstructed human epidermis with agonsitic IL-36 ligands induces S100A7 and S100A12 gene expression. S100A7 and S100A12 are genes located within the epidermal differentiation complex.

Protocol: Reconstructed human epidermis were incubated with anti-IL-36R antibodies (1.5 μg/ml) and stimulated with human recombinant IL-36γ (20 ng/ml). Recombinant human IL-113 (20 ng/mL; R & D Systems) was used as a positive control. After 24 hours in culture at 5% CO₂ and 37° C., RNA was isolated from the reconstructed human epidermis and assayed for gene expression by real-time reverse trancriptase-polymerase chain reaction. Relative expression was calculated using the 2^{-ΔΔCt} method. Samples were tested in triplicate and the average expression±standard error is shown in the table below (Table 4).

TABLE 4

| | | Mean S100A7 | Mean S100A12 |
|---|---|---|---|
| | Cytokine | Expression +/− | Expression +/− |
| Antibody | Stimulation | Standard Error | Standard Error |
| No antibody | None | 1.00 ± 0.79 | 1.00 ± 0.47 |
| 33D10 | None | 3.92 ± 0.36 | 1.93 ± 0.02 |
| No antibody | 20 ng/mL IL-1β | 76.03 ± 24.66 | 47.84 ± 9.24 |
| 33D10 | 20 ng/mL IL-1β | 95.83 ± 11.83 | 76.41 ± 6.92 |
| No antibody | 20 ng/mL IL-36γ | 19.57 ± 3.26 | 20.53 ± 5.21 |
| 33D10 | 20 ng/mL IL-36γ | 3.47 ± 1.37 | 2.01 ± 0.35 |

### Example 4: Demonstration of IL-36 Ligand/Receptor Expression by In Situ Hybridization (ISH) Techniques in Human Skin Biopsies

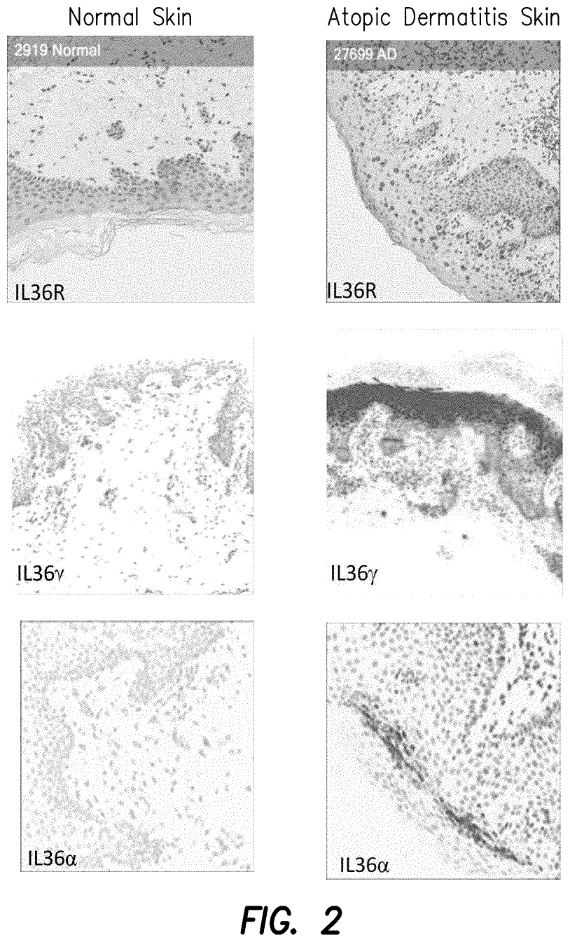

Formalin fixed paraffin embedded (FFPE) skin biopsies from atopic dermatitis and non-AtD healthy controls were purchased from a vendor and using ISH probes, stained for IL-36 α,β,γ and IL-36R. Increased expression of IL36R as well as IL36α,γ are seen in atopic dermatitis skin samples, primarily in epidermis with sporadic/rare expression in a sub population of dermal cells. See FIG. 2.

### Example 5: Phase I, Multicenter, Randomized, Double-Blind, Multiple Dose, Placebo-Controlled, Parallel-Group Study to Assess the Safety, Tolerability, Pharmacokinetics and Efficacy of a 16-Week Treatment with an Antibody of the Present Invention in Patients with Atopic Dermatitis

In this example, a compound or a product of the present invention is administered to a patient with atopic dermatitis and the safety, tolerability, pharmacokinetics and efficacy of the compound or product in patients with atopic dermatitis (AtD) is determined.

### Mode of Administration: IV, SC

### Inclusion Criteria:

Adults with chronic AtD for at least 3 years (EASI≥16, IGA≥3, 10%≥BSA)
Max Itch Intensity on NRS 3
Documented inadequate response to topical corticosteroids and able to stop TCS for at least 14 days prior to randomization

### Exclusion Criteria

Use of topical or systemic corticosteroids or other agents for AtD without appropriate wash out period
Active infection requiring antibiotic treatment
Women who are pregnant, nursing, or who plan to become pregnant while in the trial.
Severe, progressive, or uncontrolled renal, hepatic, haematological, endocrine, pulmonary, cardiac, neurologic, cerebral, or psychiatric disease, or signs and symptoms thereof.
Patient with a transplanted organ (with exception of a corneal transplant >12 weeks prior to screening) or who have ever received stem cell therapy (e.g., Prochymal).
Known history of lymphoproliferative disease, including lymphoma, or signs and symptoms suggestive of possible lymphoproliferative disease, such as lymphadenopathy and/or splenomegaly.
Any documented active or suspected malignancy or history of malignancy within 5 years prior to the screening visit, except appropriately treated basal or squamous cell carcinoma of the skin or in situ carcinoma of uterine cervix.
Patients who have previously undergone allergy immunotherapy for prevention of anaphylactic reactions.
Use of any restricted medication as specified in Table 4.2.2.1: 1 or any drug considered likely to interfere with the safe conduct of the study, as assessed by the investigator.
Plans for administration of live vaccines during the study period or within 6 weeks prior to randomisation.
History of allergy/hypersensitivity to a systemically administered biologic agent or its excipients.
Active systemic infections during the last 2 weeks (exception: common cold) prior to randomisation, as assessed by the investigator.
Chronic or relevant acute infections including human immunodeficiency virus (HIV), viral hepatitis and (or) active or latent tuberculosis (patients with a positive QuantiFERON TB test are excluded. Patients with suspected false positive or undeterminable QuantiFE RON TB result may be re-tested).
Major surgery performed within 12 weeks prior to randomisation or planned within 32 weeks after randomisation (e.g. hip replacement, aneurysm removal, stomach ligation), as assessed by the investigator.
Total white blood count (WBC)<3,000/μL, or platelets <100,000/μL or neutrophils <1,500/μL, or hemoglobin <8.5 g/dL at screening.
Aspartate aminotransferase (AST) or alanine aminotransferase (ALT)>2× the upper limit of normal, or total bilirubin >1.5× the upper limit of normal (patients with Gilbert's syndrome are not excluded) at screening.
Currently enrolled in another investigational device or drug study, or less than 30 days since ending another investigational device or drug study(s), or receiving other investigational treatment(s).
Chronic alcohol or drug abuse or any condition that, in the investigator's opinion, makes them an unreliable study subject or unlikely to complete the trial.

### Endpoints:

The following endpoints are measure for assessing safety and efficacy and/or improvement over placebo or baseline: Change in EASI score at 4 and 16 weeks; proportion of patients who attain EASI 50 at 4 and 16 weeks; proportion of patients who attain EASI 75 at 4 and 16 weeks; Change in SCORAD, Max Itch Intensity and DLQI.

### Safety Criteria:

Adverse events, vital signs, physical examination, ECG, and clinical laboratory parameters.

### Example 6: Treating Patients with AtD

In this example, an anti-IL36R antibody of the present invention is used to treat patients with AtD.

Following the administration of the anti-IL-36R antibody, safety and efficacy assessments reveal the following: At least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% of the AtD patients show improvements over baseline or as compared to placebo for the endpoints listed in Example 7 or in claims.

### Example 7: Phase IIa, Multicenter, Randomized, Double-Blind, Placebo-Controlled, Study to Evaluate the Safety, Tolerability and Efficacy of Treatment with BI 655130 (Spesolimab) in Adult Patients with Moderate to Severe Atopic Dermatitis

In this example, a compound or a product of the present invention is administered to an adult patient with moderate to severe atopic dermatitis and the following outcomes are measured.

### Primary Outcome Measures:

The percentage change from baseline in the Eczema Area and Severity Index (EASI) Score at Week 16 [Time Frame: Baseline and Week 16]

### Secondary Outcome Measures:

Number of patients with drug related Adverse Events (AEs) [Time Frame: Up to Week 44]

Absolute and percentage change from baseline in Eczema Area and Severity Index (EASI) at Week 4 [Time Frame: Baseline and Week 4]

Proportion of patients with a 50% improvement from baseline in Eczema Area and Severity Index (EASI)(EASI50) at Week 4 and 16 [Time Frame: Baseline, Week 4 and Week 16]

Proportion of patients with a 75% improvement from baseline in Eczema Area and Severity Index (EASI)(EASI75) at Week 4 and 16 [Time Frame: Baseline, Week 4 and Week 16]

Change from baseline in SCORing of Atopic Dermatitis (SCORAD) at Week 4 and 16 [Time Frame: Baseline, Week 4 and Week 16]

Proportion of patients achieving at least a 2-grade reduction from baseline to clear (0) or almost clear (1) in Investigator's Global Assessment (IGA) at Week 4 and 16 [Time Frame: Baseline, Week 4 and Week 16]

### Mode of Administration: IV, SC

### Inclusion Criteria:

Signed and dated written informed consent in accordance with Good Clinical Practice (GCP) and local legislation prior to the start of any screening procedures
Male or female patients, 18 to 75 years of age at screening
Diagnosis of atopic dermatitis for at least 1 year
Moderate to severe atopic dermatitis defined as:
   At least 10% Body Surface Area (BSA) of atopic dermatitis involvement at screening and baseline
   Eczema Area and Severity Index (EASI) of at least 12 at screening and at least 16 at baseline
   Investigator Global Assessment (IGA) of at least 3 at screening and baseline
Documented history of inadequate response to topical corticosteroid as judged by the investigator
Willing to use a standard emollient for the duration of the study
Women of childbearing potential (WOCBP) must be ready and able to use highly effective methods of birth control per ICH M3 (R2) that result in a low failure rate of less than 1% per year when used consistently and correctly. A list of contraception methods meeting these criteria is provided in the patient information.

### Exclusion Criteria:

Use of topical corticosteroids or other agents for atopic dermatitis within 7 days prior to first dose of trial treatment.
Use of systemic corticosteroids or other agents for atopic dermatitis within 4 weeks prior to first dose of trial treatment.
Women who are pregnant, nursing, or who plan to become pregnant while in the trial. Women who stop nursing before the study drug administration do not need to be excluded from participating; they should refrain from breastfeeding up to 16 weeks after the last study drug administration
Patient with a transplanted organ (with exception of a corneal transplant >12 weeks prior to screening) or who have ever received stem cell therapy (e.g., Prochymal).
Any documented active or suspected malignancy or history of malignancy within 5 years prior to the screening visit, except appropriately treated squamous cell carcinoma of the skin or in situ carcinoma of uterine cervix.
Use of any restricted medication or any drug considered likely to interfere with the safe conduct of the study, as assessed by the investigator.
History of allergy/hypersensitivity to the systemically administered trial medication agent or its excipients.
Active systemic infections (Fungal and bacterial disease) during the last 2 weeks prior to first drug administration, per investigator assessment.
Relevant chronic or acute infections (exception: common cold) including human immunodeficiency virus (HIV) or viral hepatitis. A patient can be re-screened if the patient was treated and is cured from the acute infection.
Active or Latent Tuberculosis (TB):
   Patients with active tuberculosis are excluded.
   Patients with a positive QuantiFERON TB test during screening are excluded, unless:
      Patient had previous diagnosis of active or latent TB and has completed appropriate treatment per local practice/guidelines within the last 3 years and at least 6 months before first administration of trial medication under this protocol (patients may be re-screened once to meet this criterion)
      Patients with suspected false positive or indeterminate QuantiFERON TB result may be re-tested once
      If the QuantiFERON TB test result is not available or provides indeterminate results after repeat testing: A tuberculin skin test reaction 10 mm (5 mm if receiving ≥15 mg/d prednisone or its equivalent) is considered positive and patients will be excluded.
Currently enrolled in another investigational device or drug trial, or less than 30 days since ending another investigational device or drug trial(s), or receiving other investigational treatment(s).
Evidence of a current or previous disease, medical condition (including chronic alcohol or drug abuse or any condition) other than AD, surgical procedure, psychiatric or social problems, medical examination finding (including vital signs and ECG), or laboratory value at the screening outside the reference range that in the opinion of the investigator is clinically significant and would make the study participant unreliable to adhere to the protocol, comply with all study visits/procedures or to complete the trial, compromise the safety of the patient or compromise the quality of the data.
Major surgery (major according to the investigator) performed within 12 weeks prior to first study drug administration or planned during the study (e.g. hip replacement, aneurysm removal, stomach ligation).
Severe, progressive, or uncontrolled hepatic disease, defined as >3-fold Upper Limit of Normal (ULN) elevation in AST or ALT or alkaline phosphatase, or >2-fold ULN elevation in total bilirubin.

### Results

### Summary

In the Phase IIa, randomized, double-blind, placebo-controlled trial 1368.32, 51 patients with moderate to severe atopic dermatitis were treated with either 600 mg spesolimab (33 patients) or placebo (18 patients) intravenously every 4 weeks over a period of 16 weeks (time point of primary analysis). The trial is still ongoing in an open-label extension phase, but preliminary data from the primary analysis at Week 16 are available.

The percent change from baseline in EASI score after 16 weeks of treatment showed a clinically meaningful treatment effect of 600 mg spesolimab: the adjusted mean difference to placebo was −25.6% (90% Cl −54.9%, 3.7%). The results indicate that IL-36 plays a role in AD and spesolimab could be an effective treatment option. There were not relevant differences in AE frequencies and laboratory values, spesolimab was well tolerated, and no safety signal was identified in trial 1368.32.

### Clinical Data

### Disposition and Demographics

In the Phase IIa, randomized, double-blind, placebo-controlled trial 1368.32, 51 patients with moderate to severe atopic dermatitis were entered. Patients were randomized in a 2:1 allocation ratio and received either 600 mg spesolimab (33 treated patients) or placebo (18 treated patients) intravenously every 4 weeks over a period of 16 weeks (time point of primary analysis). After 16 weeks of treatment, non-responding patients could be re-allocated to spesolimab open-label treatment for up to 16 further weeks. The trial is still ongoing in this open-label phase, but preliminary data from the primary analysis at Week 16 are available.

At the time of interim analysis, 8 patients (44.4%) in the placebo group and 22 patients (66.7%) in the spesolimab group had completed 16 weeks of double-blind treatment. The most common reasons for premature discontinuation of trial medication were adverse events (placebo: 16.7%; spesolimab: 15.2%), withdrawal by subject (placebo: 16.7%; spesolimab: 9.1%), and lack of efficacy (placebo: 11.1%; spesolimab: 3.0%).

Demographic and disease characteristics at baseline were reasonably balanced between treatment groups. About half of the patients were female (51.0%); and patients were White (47.1%), Asian (31.4%), or Black/African American (21.6%). The mean age was 39.4 years (SD 15.5 years); and the mean time since first diagnosis of AD was 20.3 years (SD 14.9 years). The baseline EASI score was 24.9 (SD 10.8), with around two-thirds of patients in a severe EASI category (60.8%) and one-third in a moderate EASI category (39.2%).

### Safety Results

Overall 51 patients with moderate to severe atopic dermatitis were randomized into this double-blind, randomized and placebo-controlled trial of spesolimab. Patients were randomized in a 2:1 allocation ratio and received either 600 mg spesolimab (33 treated patients) or placebo (18 treated patients) intravenously every 4 weeks over a period of 16 weeks (time point of primary analysis). After 16 weeks of treatment, non-responding patients could be re-allocated to spesolimab open-label treatment for up to 16 further weeks. The trial is still ongoing in this open-label phase, but preliminary data from the primary analysis at Week 16 are available.

During the double-blind treatment period, 10 out of 18 patients (55.6%) in the placebo group and 24 out of 33 patients (72.7%) in the spesolimab group reported at least 1 AE while on treatment. The frequency of patients with severe AEs (RCTC grade 3 or 4) and patients with investigator-defined drug-related AEs was higher in the placebo group than in the spesolimab group (placebo: 6 patients, 33.3%; spesolimab: 4 patients, 12.1%). Adverse events leading to discontinuation and SAEs were reported with similar frequencies in both groups (Table 5).

TABLE 5
Trial 1368.32: Adverse event overall summary -
SAF (double-blind treatment period)

| | | | Spesolimab | |
|---|---|---|---|---|
| | Placebo | | 600 mg | |
| | N (%) | | N (%) | |
| Treated patients | 18 | (100.0) | 33 | (100.0) |
| Patients with any AE | 10 | (55.6) | 24 | (72.7) |
| Patients with severe AEs | 6 | (33.3) | 4 | (12.1) |
| (RCTC grade 3 or 4) | | | | |
| Patients with investigator-defined | 6 | (33.3) | 4 | (12.1) |
| drug-related AEs | | | | |
| Patients with AEs leading to discontinuation | 3 | (16.7) | 7 | (21.2) |
| of trial drug¹ | | | | |
| Patients with investigator-confirmed AEs of | 1 | (5.6) | 0 | |
| special interest | | | | |
| Patients with other significant AEs (project | 2 | (11.1) | 5 | (15.2) |
| definition) | | | | |
| Patients with serious AEs | 1 | (5.6) | 3 | (9.1) |
| Results in Death | 0 | | 0 | |
| Is Life Threatening | 0 | | 0 | |
| Persistent or Significant Disability/Incapacity | 0 | | 0 | |
| Requires or Prolongs Hospitalization | 0 | | 2 | (6.1) |
| Congenital Anomaly or Birth Defect | 0 | | 0 | |
| Other Medically Important Serious Event | 1 | (5.6) | 1 | (3.0) |

Patients can be counted in more than 1 category
Adverse events are considered on-treatment if they started or worsened between first and last trial medication administration (plus residual effect period of 16 weeks)
¹This may also include patients with temporary discontinuation of trial drug

The most frequently reported AEs were “atopic dermatitis” (placebo: 7 patients, 38.9%; spesolimab: 9 patients, 27.3%), “folliculitis” (placebo: 2 patients, 11.1%; spesolimab: 3 patients, 9.1%), and “upper respiratory tract infection” (placebo: 0 patients; spesolimab: 3 patients, 9.1%). All other AEs were reported for not more than 2 patients per treatment group (Table 6).

TABLE 6
Trial 1368.32: Frequency of patients with adverse
events, by PTs reported for more than 1 patient
overall - SAF (double-blind treatment period)

| | |
|---|---|
| | Spesolimab |
| Placebo | 600 mg |
| N (%) | N (%) |

| | | | | |
|---|---|---|---|---|
| Treated patients | 18 | (100.0) | 33 | (100.0) |
| Patients with any AE | 11 | (61.1) | 24 | (72.7) |
| Atopic dermatitis | 7 | (38.9) | 9 | (27.3) |
| Folliculitis | 2 | (11.1) | 3 | (9.1) |

| | | | |
|---|---|---|---|
| Upper respiratory tract infection | 0 | 3 | (9.1) |

| | | | | |
|---|---|---|---|---|
| Nasopharyngitis | 1 | (5.6) | 2 | (6.1) |
| Nausea | 1 | (5.6) | 1 | (3.0) |
| Staphylococcal skin infection | 1 | (5.6) | 1 | (3.0) |

| | | | |
|---|---|---|---|
| Anxiety | 2 | (11.1) | 0 |

Patients can be counted in more than 1 category
Adverse events are considered on-treatment if they started or worsened between first and last trial medication administration (plus residual effect period of 16 weeks)

SAEs were reported for 1 patient in the placebo group (5.6%) and 3 patients in the spesolimab group (9.1%).

Grouped PTs related to hypersensitivity reaction (using MedDRA SMQs) were reported for 8 patients (44.4%) in the placebo group and 11 patients (33.3%) in the spesolimab group, and were mainly driven by atopic dermatitis (placebo: 7 patients, 38.9%; spesolimab: 9 patients, 27.3%).

There were no clinically relevant abnormalities on treatment with spesolimab with respect to safety laboratory and vital signs.

In conclusion, there were not relevant differences in AE frequencies and laboratory values, spesolimab was well tolerated, and no safety signal was identified in trial 1368.32.

### Efficacy Results

In the Phase IIa, randomized, double-blind, placebo-controlled trial 1368.32, 51 patients with moderate to severe atopic dermatitis were treated with either 600 mg spesolimab (33 patients) or placebo (18 patients) intravenously every 4 weeks over a period of 16 weeks (time point of primary analysis). The trial is still ongoing in an open-label extension phase, but preliminary data from the primary analysis at Week 16 are available.

The baseline disease characteristics were generally comparable between the 2 treatment groups. EASI score was 24.9 (SD 10.8), with around two-thirds of patients in a severe EASI category (60.8%) and one-third in a moderate EASI category (39.2%). The mean time since first diagnosis of AD was 20.3 years (SD 14.9 years).

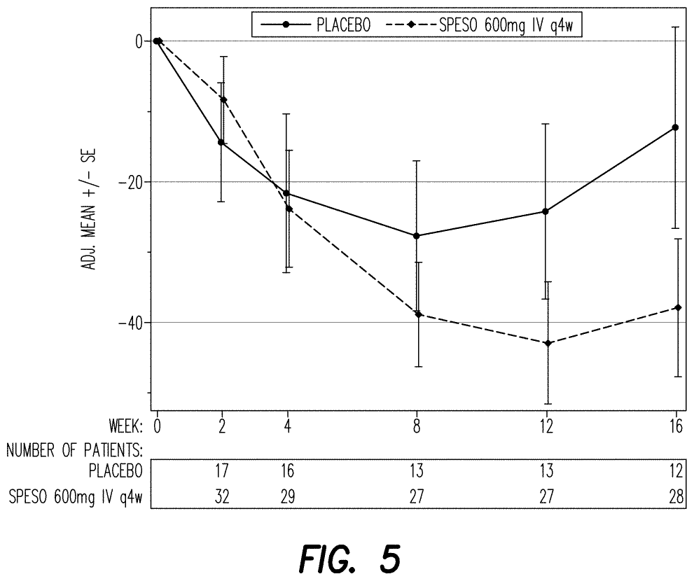

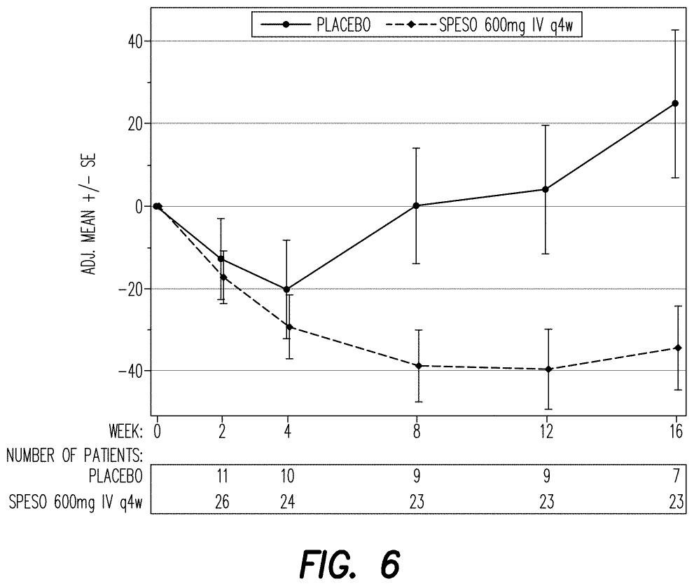

In the placebo group, the mean EASI score decreased at the beginning of the trial, but increased again from Week 8, while the mean EASI score in the spesolimab treatment group decreased continuously. Taken together, the trial showed a meaningful treatment effect from Week 8 onwards (FIG. 5).

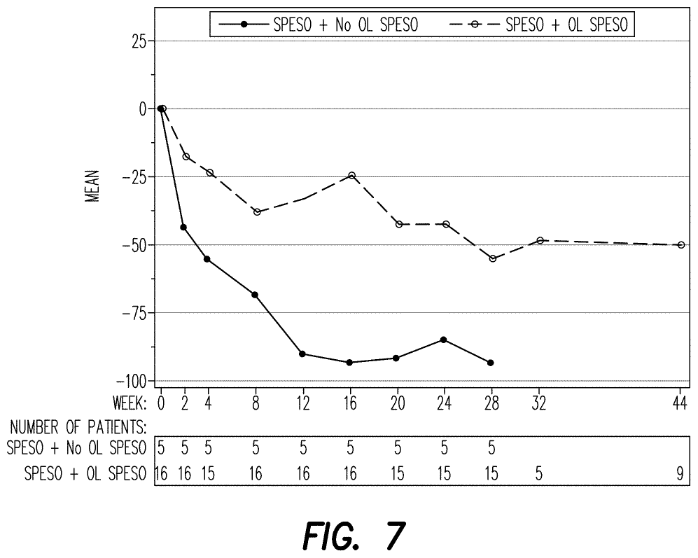

The percent change from baseline in EASI score after 16 weeks of treatment also showed a clinically meaningful, but not statistically significant treatment effect of 600 mg spesolimab: the adjusted mean difference to placebo was −25.6% (90% Cl −54.9%, 3.7%). (FIG. 5) Consistent results were observed in available secondary endpoints and sensitivity analyses. For instance, in a sensitivity analysis excluding data after concomitantly restricted steroid medication use, the adjusted mean difference to placebo was −48.3% (90% Cl 82.8%, −13.9%). (FIG. 6) In a sensitivity analysis including only patients who had completed the double-blind treatment period as planned, the adjusted mean difference to placebo was −45.6% (90% Cl −82.6%, −8.6%). Although not a formal endpoint, patients were followed up for efficacy until the end of the trial. The 5 patients initially randomized to spesolimab who were responders, achieved EASI75 at Week 16, and did therefore not receive open-label spesolimab treatment from Week 16 maintained their EASI 50 response until Week 28. (FIG. 7) Of the 16 patients initially randomized to spesolimab and who were non-responders, did not achieve EASI75 at Week 16, most showed additional reduction in EASI during the re-allocation period. In the re-allocation period, patients were put either on open label spesolimab or on ‘no drug’ depending on whether or not a patient was EASI75 responder at Week 16. Overall, the results indicate that IL-36 plays a role in AD and spesolimab could be an effective treatment option for patients. Considering the small sample size and the limitations of the trial design, the response of patients, who had responded after initial or open-label spesolimab treatment, was sustained until the end of trial.

The results in secondary endpoint measures are the following at Week 16. In the secondary endpoints EASI50 and EASI75 the risk difference (i.e., the improvements observed in the spesolimab patients as compared to the placebo patients) (90% Cl) was 18.3 (3.3, 33.4) and 7.6 (−6.8, 22.1), respectively. For the secondary endpoint SCORAD, the adjusted mean difference (i.e., improvement) to placebo in SCORAD percent change from baseline after 16 weeks of treatment was −14.9 (90% Cl −35.2, 5.5). For the secondary endpoint IGA (IGA 0 or 1 with grades reduction) the risk difference (i.e., improvement) (90% Cl) was 6.1 (−3.5, 15.7). For pruritus, as a subendpoint of the SCORAD, the adjusted mean difference (i.e., improvement) to placebo in percent change from baseline in pruritus VAS after 16 weeks of treatment was −20.9 (90% Cl −44.5, 2.7), excluding patients who had a score of less than 4 at baseline. In the further endpoint DLQI, the adjusted mean difference (i.e., improvement) to placebo in DLQI percent change from baseline after 16 weeks of treatment was −39.8 (90% Cl −106.4, 26.8).

### Example 8: Treating Adult Patients with AtD

In this example, an anti-IL36R antibody of the present invention is used to treat adult patients with AtD.

Following the administration of a compound or product of the present invention according to Example 7, at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% of the AtD patients show improvements over baseline or as compared to placebo for the outcome measures listed in Example 7 or in claims.

### Example 9. Efficacy and Safety of Spesolimab (BI 655130), an Anti-Interleukin-36 Receptor Antibody, in Patients with Moderate to Severe Atopic Dermatitis

Introduction & Objectives: Atopic dermatitis (AD) is a relapsing skin disease characterized by chronic inflammation and unrelenting intense pruritus. The interleukin-36 (IL-36) pathway is believed to play a key role in the pathophysiology of AD. Here, we report the efficacy and safety of spesolimab, an anti-IL-36 receptor monoclonal antibody in a Phase IIa, proof-of-concept study (NCT03822832) in patients with moderate to severe AD.

Material & Methods: In this placebo-controlled, multicenter study, patients were screened and randomized 2:1 to receive 600 mg spesolimab or placebo intravenously (every 4 weeks, up to Week 12) and evaluated after 16 weeks of double-blind treatment. Patients who responded (achieved a 75% reduction in Eczema Area and Severity Index [EASI 75] score compared with baseline) were followed for an additional 12 weeks. Patients who did not respond were reallocated to open-label 600 mg spesolimab for a further 16 weeks. The primary endpoint was percentage change from baseline in EASI score at Week 16. Secondary endpoints included change from baseline in EASI score at Week 4, the proportion of patients with EASI 50 and EASI 75 at Weeks 4 and 16, and occurrence of adverse events (AEs).

Results: In total, 71 patients were screened and 51 were randomized; 30 patients completed the 16-week double-blind treatment period, 66.7% (n=22/33) in the spesolimab arm and 44.4% (n=8/18) in the placebo arm. The percentage change from baseline in EASI score at Week 16 was −37.9% (standard error [SE] 9.8) for spesolimab and −12.3% (SE 14.3) for placebo; the adjusted mean difference (90% confidence interval [Cl]) between spesolimab and placebo was −25.6% (−54.9%, 3.7%; p=0.1492). In a sensitivity analysis that included only patients who had completed the double-blind treatment period, the adjusted mean difference between spesolimab and placebo was −45.6% (90% Cl −82.6%, −8.6%). The adjusted mean percentage change from baseline in EASI at Week 4 for spesolimab versus placebo was −2.2% (90% Cl −25.8%, 21.4%). The proportion of patients achieving EASI 50 at Week 16 was 30.3% for spesolimab (n=10/33) and 5.6% for placebo (n=1/18), with a difference of 24.1% (90% Cl 8.6%, 39.6%). Similarly, the proportion of patients achieving EASI 75 at Week 16 was 15.2% for spesolimab (n=5/33) and 5.6% for placebo (n=1/18), with a difference of 9.4% (90% Cl −4.1%, 22.9%). The proportion of patients with any AE was 72.7% (n=24/33) and 55.6% (n=10/18) in the spesolimab and placebo groups, respectively; of these, 15.2% (n=5/33) and 16.7% (n=3/18) discontinued because of AEs. Serious AEs were reported in 9.1% (n=3/33) and 5.6% (n=1/18) of patients in the spesolimab and placebo groups, respectively.

Conclusions: This proof-of-concept study showed a clinically meaningful, although not statistically significant, change in the primary endpoint of percentage change from baseline in EASI score after 16 weeks of treatment with spesolimab. Consistent results were observed in the secondary endpoints between study arms. Safety data in this trial were consistent with previous trials conducted with spesolimab. Further investigation is needed to confirm the role of IL-36 receptor inhibition in patients with moderate to severe AD.

While certain aspects and embodiments of the invention have been described, these have been presented by way of example only, and are not intended to limit the scope of the invention. Indeed, the novel methods and systems described herein may be embodied in a variety of other forms without departing from the spirit thereof. The accompanying claims and their equivalents are intended to cover such forms or modifications as would fall within the scope and spirit of the invention.

All patents and/or publications including journal articles cited in this disclosure are expressly incorporated herein by reference.

1. A method for treating atopic dermatitis (AtD) in a subject, comprising administering to the subject a dosage regimen of an anti-interleukin-36 receptor (anti-IL-36R) antibody.

2. The method of claim 1, wherein the dosage regimen comprises: (a) subcutaneous administrations of one or more doses of 300 mg or one or more doses of 600 mg each of the anti-IL-36R antibody once every week (qw), once every 2 weeks (q2w), once every 4 weeks (q4w), once every 6 weeks (q6w) or once every 8 weeks (q8w); or (b) subcutaneous administrations of the anti-IL-36R antibody in an initial dose and a subsequent dose; wherein the initial dose comprises (i) one or more doses of 150 mg each of the anti-IL-36R antibody administered daily for 2 weeks; or (ii) one or more doses of 300 mg each of the anti-IL-36R antibody administered daily for 2 weeks; or (iii) one or more doses of 600 mg each of the anti-IL-36R antibody administered twice, three times or four times in 4 weeks or administered twice per week for 2 weeks, or administered twice per week for 3 weeks, or administered twice per week for 4 weeks; or (iv) one dose of 1200 mg of the anti-IL-36R antibody administered once; or (v) two doses of 1200 mg each of the anti-IL-36R antibody administered twice in three weeks; and wherein the subsequent dose includes: (i) one or more doses of 300 mg each of the anti-IL-36R antibody administered q2w, q4w, q6w or q8w; or (ii) one or more doses of 600 mg each of the anti-IL-36R antibody administered q2w, q4w, q6w or q8w.

3. The method of claim 2, wherein the administration of the subsequent dose is between 2 to 4 weeks or 2 weeks or 4 weeks after the administration of the last initial dose.

4. The method of claim 1, wherein the anti-IL-36R antibody comprises:
I. a) a light chain variable region comprising the amino acid sequence of SEQ ID NO: 26 (L-CDR1); the amino acid sequence of SEQ ID NO: 102 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
II. a) a light chain variable region comprising the amino acid sequence of SEQ ID NO: 26 (L-CDR1); the amino acid sequence of SEQ ID NO: 103 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
III. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
IV. a) a light chain variable region comprising the amino acid sequence of SEQ ID NO: 26 (L-CDR1); the amino acid sequence of SEQ ID NO: 105 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
V. a) a light chain variable region comprising the amino acid sequence of SEQ ID NO: 26 (L-CDR1); the amino acid sequence of SEQ ID NO: 106 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
VI. a) a light chain variable region comprising the amino acid sequence of SEQ ID NO: 26 (L-CDR1); the amino acid sequence of SEQ ID NO: 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
VII. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 141 (H-CDR1); the amino acid sequence of SEQ ID NO: 62, 108, 109, 110, 111 or 142 (H-CDR2); the amino acid sequence of SEQ ID NO: 72 (H-CDR3).

5. The method claim 1, wherein the anti-IL-36R antibody comprises:
(i) a light chain variable region comprising the amino acid sequence of SEQ ID NO: 77; and a heavy chain variable region comprising the amino acid sequence of SEQ ID NO: 87; or
(ii) a light chain variable region comprising the amino acid sequence of SEQ ID NO: 77; and a heavy chain variable region comprising the amino acid sequence of SEQ ID NO: 88; or
(iii) a light chain variable region comprising the amino acid sequence of SEQ ID NO: 77; and a heavy chain variable region comprising the amino acid sequence of SEQ ID NO: 89; or
(iv) a light chain variable region comprising the amino acid sequence of SEQ ID NO: 80; and a heavy chain variable region comprising the amino acid sequence of SEQ ID NO: 87; or
(v) a light chain variable region comprising the amino acid sequence of SEQ ID NO: 80; and a heavy chain variable region comprising the amino acid sequence of SEQ ID NO: 88; or
(vi) a light chain variable region comprising the amino acid sequence of SEQ ID NO: 80; and a heavy chain variable region comprising the amino acid sequence of SEQ ID NO: 89; or
(vii) a light chain variable region comprising the amino acid sequence of SEQ ID NO: 85; and a heavy chain variable region comprising the amino acid sequence of SEQ ID NO: 100; or
(viii) a light chain variable region comprising the amino acid sequence of SEQ ID NO: 85; and a heavy chain variable region comprising the amino acid sequence of SEQ ID NO:101; or
(ix) a light chain variable region comprising the amino acid sequence of SEQ ID NO: 86; and a heavy chain variable region comprising the amino acid sequence of SEQ ID NO: 100; or
(x) a light chain variable region comprising the amino acid sequence of SEQ ID NO: 86; and a heavy chain variable region comprising the amino acid sequence of SEQ ID NO:101.

6. The method of claim 1, wherein the anti-IL-36R antibody comprises:
i. a light chain comprising the amino acid sequence of SEQ ID NO: 115; and a heavy chain comprising the amino acid sequence of SEQ ID NO: 125; or
ii. a light chain comprising the amino acid sequence of SEQ ID NO: 115; and a heavy chain comprising the amino acid sequence of SEQ ID NO: 126; or
iii. a light chain comprising the amino acid sequence of SEQ ID NO: 115; and a heavy chain comprising the amino acid sequence of SEQ ID NO: 127; or
iv. a light chain comprising the amino acid sequence of SEQ ID NO: 118; and a heavy chain comprising the amino acid sequence of SEQ ID NO: 125; or
v. a light chain comprising the amino acid sequence of SEQ ID NO: 118; and a heavy chain comprising the amino acid sequence of SEQ ID NO: 126; or
vi. a light chain comprising the amino acid sequence of SEQ ID NO: 118; and a heavy chain comprising the amino acid sequence of SEQ ID NO: 127; or
vii. a light chain comprising the amino acid sequence of SEQ ID NO: 123; and a heavy chain comprising the amino acid sequence of SEQ ID NO: 138; or
viii. a light chain comprising the amino acid sequence of SEQ ID NO: 123; and a heavy chain comprising the amino acid sequence of SEQ ID NO: 139; or
ix. a light chain comprising the amino acid sequence of SEQ ID NO: 124; and a heavy chain comprising the amino acid sequence of SEQ ID NO: 138.

7. The method of claim 1, wherein a second therapeutic agent is administered to the subject before, after, or concurrent with the anti-IL-36R antibody.

8. The method of claim 7, wherein the second therapeutic agent is selected from the group consisting of an anti-bacterial agent, an anti-viral agent, an anti-fungal agent, an anti-IL-36R antibody, an IgE inhibitor, a corticosteroid, a non-steroid anti-inflammatory drug (NSAID), an IL-4R antagonist, and IFN-γ.

9. The method of claim 1, wherein the treatment results in an improvement in the subject; wherein the improvement is determined by an endpoint selected from the group consisting of: (i) positive changes in Eczema Area and Severity Index (EASI) score at 16 weeks after the treatment; (ii) attaining an EASI 50 at 16 weeks after the treatment; (iii) attaining an EASI 75 at week 16 after the treatment; and (iv) a positive change in Scoring Atopic Dermatitis (SCORAD), Max Itch Intensity or Dermatology Life Quality Index (DLQI).

10. The method according claim 1, wherein the treatment results in one or more of the following outcomes in the subject as compared to the subject's conditions at baseline or before the treatment or as compared to placebo:
a. at least 10% improvement in EASI score at week 16 after the treatment; or at least 10 percentage point difference in percentage change from baseline in EASI score at week 16; or
b. at least 10% improvement in EASI 50 at week 16 after the treatment; or at least 10 percentage point difference in EASI50 response rate at week 16; or
c. at least 5% improvement in EASI 75 at week 16 after the treatment; or at least 5 percentage point difference in EASI75 response rate at week 16; or
d. at least 10% improvement in SCORAD, Max Itch Intensity and DLQI at week 16 after the treatment; or at least 10 percentage point difference in percentage change from baseline in SCORAD, Max Itch Intensity and DLQI at week 16; or
e. at least 10% improvement in absolute and percentage change in Eczema Area and Severity Index (EASI) at week 44 after the treatment; or at least 10 percentage point difference in percentage change from baseline in Eczema Area and Severity Index (EASI) at week 44; or
f. at least 5% improvement in Investigator's Global Assessment (IGA) at week 16 after the treatment; or at least 5 percentage point difference in IGA rate at week 16.
